(19) United States
(12) Patent Application Publication
(10) Pub. No.: US 2026/0298940 A1
(43) Pub. Date: Oct. 1, 2026

(54) METHODS FOR DETECTING AN ANTI-DRUG ANTIBODY (ADA) AGAINST AN ANTI-TNF ALPHA ANTIBODY

(71) Applicant: Xentria, Inc., Chicago, IL (US)

(72) Inventor: Thomas Matthews, Chicago, IL (US)

(21) Appl. No.: 19/476,457

(22) PCT Filed: Apr. 19, 2024
(86) PCT No.: PCT/US2024/025346

§ 371 Date: Oct. 17, 2025

Related U.S. Application Data
(60) Provisional application No. 63/460,821, filed on Apr. 20, 2023.
(60) Provisional application No. 63/461,131, filed on Apr. 21, 2023.
(60) Provisional application No. 63/508,436, filed on Jun. 15, 2023.

(51) Int. Cl. G01N 33/68; G01N 21/76; G01N 33/543

(52) U.S. Cl. CPC G01N 33/6854; G01N 21/76; G01N 33/543; G01N 2333/525; G01N 2470/10

(57) ABSTRACT

The present technology includes methods for detecting an anti-drug antibody (ADA) that binds an anti-TNFα antibody (target antibody) in a sample comprising incubating the sample with a composition comprising an affinity antibody and a detection antibody, and isolating and detecting the ADAs that form a complex with the affinity antibody and the detection antibody. The technology also includes methods for determining the ADA concentration in a sample and confirming that a sample comprises ADAs. The technology also includes a kit for performing any of the methods of the present technology.

### CROSS-REFERENCE TO RELATED APPLICATION(S)

This application claims the benefit of U.S. Provisional Patent Application No. 63/460,821, filed on Apr. 20, 2023, U.S. Provisional Patent Application No. 63/461,131, filed on Apr. 21, 2023, and U.S. Provisional Patent Application No. 63/508,436, filed on Jun. 15, 2023. The contents of each of these provisional applications are incorporated by reference in their entirety.

### SEQUENCE LISTING

This application contains an ST.26 compliant Sequence Listing, which was submitted in xml format via Patent Center and is hereby incorporated by reference in its entirety. The .xml copy, created on Apr. 16, 2024, is named 140505_8005_WO00_SL.xml and is 20,939 bytes in size.

### TECHNICAL FIELD

The present technology includes an in vitro assay for identifying antibodies, such as an anti-drug antibody (ADA) that bind an anti-TNFα antibody.

### SUMMARY

In some embodiments, the present technology generally relates to methods for detecting an anti-drug antibody (ADA) that binds a target antibody comprising an anti-TNFα antibody in a sample comprising: (i) incubating the sample with a composition comprising: (a) a first affinity antibody comprising an anti-TNFα antibody conjugated to a first affinity label, and (b) a detection antibody comprising an anti-TNFα antibody conjugated to a detection label; (ii) isolating ADAs that form a complex with both the first affinity antibody and the detection antibody, wherein the ADA is isolated by contacting the complex with a second affinity label that binds to the first affinity label; and (iii) detecting the ADA by measuring a level of the detection label above a predetermined cut-point.

In some embodiments, isolating the ADAs comprises: (a) providing a solid phase comprising the second affinity label; (b) contacting the sample and the composition with the solid phase; and (c) washing the solid phase.

In some embodiments, the target antibody, the affinity antibody, and/or the detection antibody comprise a chimeric human-murine monoclonal IgG1-kappa antibody. In some embodiments, the target antibody, the affinity antibody, and/or the detection antibody comprise SEQ ID NOs: 1 and 4.

In some embodiments, the solid phase is a plate.

In some embodiments, the first affinity label is biotin. In some embodiments, the second affinity label is streptavidin.

In some embodiments, the detection antibody comprises a sulfo-Tag labeled anti-TNFα antibody.

In some embodiments, measuring the level of the detection label comprises: (1) contacting the solid phase with a marker configured to produce a chemiluminescent or electrochemiluminescent signal when the marker is proximal to the detection label; and (2) detecting the chemiluminescent or electrochemiluminescent signal.

In some embodiments, the marker is tripropylamine (TPA), and measuring the level of the detection label further comprises applying an electrical current to the marker to produce the chemiluminescent or electrochemiluminescent signal.

In some embodiments, the solid phase is blocked with a blocking buffer before the sample and the composition are contacted with the solid phase.

In some embodiments, the methods further comprise performing steps (i) to (iii) on a control sample. In some embodiments, the control sample is incubated with the composition and contacted with the second affinity label.

In some embodiments, the predetermined cut-point corresponds to a level with a 5% false-positive rate. In some embodiments, the 5% false-positive rate is determined by measuring the detection label in a plurality of control samples and calculating the 5% false-positive rate.

In some embodiments, the chemiluminescent or electrochemiluminescent signal is measured as electrochemiluminescence (ECL).

In some embodiments, the predetermined cut-point is an ECL value of at least 1. In some embodiments, the predetermined cut-point is an ECL value of about 1.10.

In some embodiments, the methods have a sensitivity that is less than 6 ng/ml. In some embodiments, the sensitivity is less than 4 ng/mL. In some embodiments, the methods have a drug tolerance of about 100 ng/mL.

In some embodiments, the present technology generally relates to methods for detecting an ADA that binds a target antibody comprising an anti-TNFα antibody in a sample comprising: (i) contacting a sample with one or more anti-TNFα conjugates to form a complex between the ADA and the anti-TNFα conjugates; and (ii) measuring a level of anti-TNFα conjugates in the complex; wherein the methods detect the ADA with a sensitivity less than 5 ng/mL.

In some embodiments, the one or more anti-TNFα conjugates comprise an affinity antibody comprising an anti-TNFα antibody conjugated to an affinity label.

In some embodiments, the one or more anti-TNFα conjugates comprise a detection antibody comprising an anti-TNFα antibody conjugated to a detection label.

In some embodiments, the affinity label comprises biotin. In some embodiments, the detection label comprises a sulfo-Tag.

In some embodiments, the methods detect the ADA with a sensitivity less than 3 ng/ml. In some embodiments, the methods detect the ADA with a sensitivity of 2.77 ng/ml or less.

In some embodiments, the present technology generally relates to methods for estimating a quantity of an ADA that binds a target antibody comprising an anti-TNFα antibody in a sample comprising: (i) incubating the sample with a composition comprising: (a) an affinity antibody comprising an anti-TNFα antibody conjugated to a first affinity label, and (b) a detection antibody comprising an anti-TNFα antibody conjugated to a detection label; (ii) isolating ADAs that form a complex with both the affinity antibody and the detection antibody, wherein the ADA is isolated by contacting the complex with a second affinity label that binds to the first affinity label; (iii) detecting the ADA by measuring a level of the detection label above a predetermined cut-point; (iv) performing steps (i)-(iii) on a known sample having a known concentration of ADA; and (v) comparing the level of the detection label in the sample to the level of the detection label in the known sample.

In some embodiments, the known sample is serially diluted into a plurality of titer samples, wherein comparing the level of the detection label in the sample to the level of the detection label in the known sample comprises generating a curve of the levels of the detection label in each of the titer samples and comparing the detection label in the sample to the curve.

In some embodiments, the present technology generally relates to a methods of confirming that an ADA binds to a target antibody comprising an anti-TNFα antibody in a sample by performing a competitive binding assay comprising (i) incubating the sample with a composition comprising: (a) a first affinity antibody comprising an anti-TNFα antibody conjugated to a first affinity label, and (b) a detection antibody comprising an anti-TNFα antibody conjugated to a detection label; (ii) isolating ADAs that form a complex with both the first affinity antibody and the detection antibody, wherein the ADA is isolated by contacting the complex with a second affinity label that binds to the first affinity label; and (iii) detecting the ADA by measuring a level of the detection label above a predetermined cut-point, wherein the composition further comprises an unlabeled anti-TNFα antibody.

In some embodiments, the ADA is confirmed in the sample by measuring a level of the detection label that is lower by a predetermined factor than the level of the detection label when the unlabeled anti-TNFα antibody is not present.

In some embodiments, the present technology generally relates to kits for detecting an ADA that binds a target antibody comprising an anti-TNFα antibody in a sample comprising: (i) an affinity antibody comprising an anti-TNFα antibody conjugated to a first affinity label; (ii) a detection antibody comprising an anti-TNFα antibody conjugated to a detection label; and (iii) a solid phase comprising a second affinity label, wherein the second affinity label binds to the first affinity label.

In some embodiments, the solid phase is a plate. In some embodiments, the second affinity label is streptavidin. In some embodiments, the first affinity label is biotin. In some embodiments, the detection antibody comprises a sulfo-Tag labeled anti-TNFα antibody.

In some embodiments, the kit further comprises a marker configured to produce a chemiluminescent or electrochemiluminescent signal where the marker is proximal to the detection label. In some embodiments, the marker is tripropylamine (TPA).

In some embodiments, the kit further comprises a blocking buffer. In some embodiments, the kit further comprises a wash buffer.

In some embodiments, the present technology generally relates to a kit for estimating a quantity of an ADA that binds a target antibody comprising an anti-TNFα antibody in a sample comprising: (i) an affinity antibody comprising an anti-TNFα antibody conjugated to a first affinity label; (ii) a detection antibody comprising an anti-TNFα antibody conjugated to a detection label; (iii) a solid phase comprising a second affinity label, wherein the second affinity label binds to the first affinity label; and (iv) a positive control. In some embodiments, the positive control is a plurality of titer samples.

In some embodiments, the present technology generally relates to a kit for confirming that an ADA binds a target antibody comprising an anti-TNFα antibody comprising: (i) an affinity antibody comprising an anti-TNFα antibody conjugated to a first affinity label; (ii) a detection antibody comprising an anti-TNFα antibody conjugated to a detection label; (iii) an unlabeled anti-TNFα antibody; and (iv) a solid phase comprising a second affinity label, wherein the second affinity label binds to the first affinity label.

### BACKGROUND

Anti-TNFα (Tumor Necrosis Factor alpha) antibodies are novel biologics that may be used for the treatment of various diseases. One hurdle to the development and implementation of anti-TNFα antibodies as a treatment is the potential for a subject administered such anti-TNFα antibodies to exhibit an immune response against them. A subject's immunogenicity toward the anti-TNFα antibodies, particularly the subject's development of anti-drug antibodies (ADAs) that bind the anti-TNFα antibody, would thereby reduce efficacy of the anti-TNFα antibody to treat the subject's disease. To advance the development of treatments that use anti-TNFα antibodies, there is need for assays that reliably determine the presence of ADAs and amounts thereof in subjects receiving or having been administered the anti-TNFα antibody.

### BRIEF DESCRIPTION OF THE DRAWINGS

FIG. 1 shows a flow diagram of an example method according to the present technology.

FIGS. 2A and 2B show normality of a set of values of percent inhibition that were tested according to the Shapiro-Wilk W Test in an example screening and titration data normality test (FIG. 2A) and in an example confirmatory data normality test (FIG. 2B) according to the methods of the present technology.

### DETAILED DESCRIPTION

The present technology comprises methods for detecting an anti-drug antibody (ADA) against a target antibody. The target antibody may comprise an anti-TNFα antibody. In some embodiments, the anti-TNFα antibody is a chimeric human-murine monoclonal IgG1-kappa antibody. In some embodiments, the target antibody comprises heavy chain complementarity-determining regions (CDRs) of SEQ ID NOs: 9-11 and light chain CDRs of SEQ ID NOs: 12-14. In some embodiments, the target antibody comprises heavy chain CDRs of SEQ ID NOs: 15-17 and light chain CDRs of SEQ ID NOs: 18-20. In some embodiments, the target antibody comprises a heavy chain variable region of SEQ ID NO: 2 and a light chain variable region of SEQ ID NO: 5. In some embodiments, the target antibody comprises a heavy chain of SEQ ID NO: 1 and a light chain of SEQ ID NO: 4. The present technology also includes a kit for detecting the target antibody in the sample according to the methods of the present technology.

While the present technology is capable of being embodied in various forms, the description below of several embodiments is made with the understanding that the present technology is to be considered as an exemplification of the technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range, as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

To the extent any materials incorporated by reference herein conflict with the present technology, the present technology controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

As used herein, an “affinity antibody” comprises an anti-TNFα antibody. In some embodiments, the affinity antibody comprises the target antibody. The affinity antibody may also comprise an affinity label that has a high affinity with a separate label. For example, the affinity label may be a first affinity label (i.e., biotin) that has a high affinity with a second affinity label (i.e., streptavidin). In some embodiments, the affinity antibody comprises an anti-TNFα antibody and an affinity label. In some embodiments, the affinity antibody comprises the target antibody and an affinity label.

As used herein, a “detection antibody” comprises a TNFα antibody. In some embodiments, the detection antibody comprises the target antibody. The detection antibody may also comprise a detection label that may be directly or indirectly detected. In some embodiments, the detection antibody comprises an anti-TNFα antibody and a detection label. In some embodiments, the detection antibody comprises the target antibody and a detection label.

In some embodiments, the method for detecting an ADA that binds the target antibody in a sample comprises: (i) contacting a sample with one or more anti-TNFα conjugates to form a complex between the ADA and the anti-TNFα conjugates; and (ii) measuring a level of anti-TNFα conjugates in the complex; wherein the method detects the ADA with a sensitivity less than 5 ng/mL. In some embodiments, the one or more anti-TNFα conjugates comprise a detection antibody. In some embodiments, measuring a level of anti-TNFα conjugates comprises measuring the level of the detection label. In some embodiments, the one or more anti-TNFα conjugates comprise an affinity antibody.

As used herein, a “screening assay” generally refers to a method of detecting an ADA that binds the target antibody. For example, a screening assay includes a method of detecting an ADA that binds a target antibody comprising: (i) incubating the sample with a composition comprising: (a) an anti-TNFα antibody conjugated to a first affinity label (affinity antibody), and (b) an anti-TNFα antibody conjugated to a detection label (detection antibody); (ii) isolating ADAs that form a complex with both the affinity antibody and the detection antibody, wherein the ADA is isolated by contacting the complex with a second affinity label that binds to the first affinity label; and (iii) detecting the ADA by measuring a level of the detection label above a predetermined cut-point. A screening assay may also include a method of detecting an ADA comprising: (i) contacting a sample with one or more anti-TNFα conjugates to form a complex between the ADA and the anti-TNFα conjugates; and (ii) measuring a level of anti-TNFα conjugates in the complex; wherein the method detects the ADA with a sensitivity less than 5 ng/mL.

In some embodiments, measuring the level of the detection label comprises contacting the solid phase with a marker configured to produce a chemiluminescent or electrochemiluminescent (ECL) signal when the marker is proximal to the detection label and detecting the chemiluminescent or ECL signal. As used herein, the marker is “proximal” to the detection label when the marker is at a distance from the detection label that is small enough to cause a reaction that produces a chemiluminescent or ECL signal. In some embodiments, the ECL is produced when the marker is proximal to the detection label and an electrode.

The sample may be a sample obtained from a mammal, including human, rat, mouse, rabbit, or other animal. In some embodiments, the sample is a blood, plasma, or serum sample. In some embodiments, the sample is plasma. In some embodiments, the sample is serum. In some embodiments, the sample is pre-processed before the ADA is detected according to the methods of the present technology. As used herein, “pre-processing” includes extracting tissue from a subject and identifying the sample (e.g., drawing a blood sample and identifying or isolating a plasma or serum sample, or separating the blood sample into the plasma sample and/or the scrum sample).

### Anti-Drug Antibodies (ADAs)

During or after administration of a drug (e.g., a therapeutic drug) to a subject, the subject's immune system may respond by generating anti-drug antibodies (ADAs). ADAs may bind at least a portion of the drug, thereby sequestering, neutralizing, and/or reducing efficacy of the drug.

ADAs may be naturally produced as part of a subject's innate immune response. The ADA may be a natural antibody produced by the subject, for example by lymphocytes using variable (diversity) joining (V (D) J) recombination. For example, the ADA may be an immunoglobulin. In some embodiments, the ADA is an IgG, IgA, IgE, monoclonal antibody, polyclonal antibody, or any other kind of antibody that is capable of binding to the target antibody.

### Target Antibodies

During or after administration of a drug (i.e., a therapeutic drug), ADAs which bind at least a portion of the drug may develop. In some embodiments, the drug comprises antibodies, and, when ADAs develop, the ADAs may bind the drug antibodies. In some embodiments, the drug antibodies comprise an anti-TNFα antibody.

Presence of ADAs and/or binding of ADAs to the drug antibodies may be detected using antibodies which are the same as or mimic the drug antibodies (i.e., “target antibodies”). In some embodiments, the target antibodies comprise one or more anti-TNFα antibodies. The anti-TNFα antibodies are antibodies which bind at least a portion of a TNFα protein or peptide.

In some embodiments, the target antibodies comprise a monoclonal antibody.

In some embodiments, the target antibodies of the present technology are chimeric antibodies. Nonlimiting examples of chimeric antibodies include human-murine antibodies, human-rat antibodies, human-hamster antibodies, human-rabbit antibodies, human-bovine antibodies, and human-goat antibodies. In some embodiments, the chimeric antibodies comprise a humanized antibody (e.g., a full humanized antibody or a partially humanized antibody).

In some embodiments, the target antibodies (e.g., anti-TNFα antibodies) of the present technology may comprise an isotype that influences functional properties and/or structural features of the antibody. In some embodiments the target antibodies comprise an isotype selected from the group consisting of IgG (e.g., IgG1, IgG2, IgG3, IgG4), IgM, IgA, IgD, and IgE. The light chain may comprise isotype kappa (κ) or isotype lambda (λ). In some embodiments, the target antibody comprises an IgG1-kappa antibody.

### Target Antibody Sequences

### Heavy Chain:

The target antibodies of the present technology comprise a heavy chain having an amino acid sequence that is about 75%, 80%, 85%, 90%, 95%, 99%, or 100% identical to SEQ. ID NO: 1.

The target antibodies of the present technology comprise a heavy chain having an amino acid sequence that is at least 75%, 80%, 85%, 90%, 95%, 99%, or 100% identical to SEQ. ID NO: 1.

The target antibodies of the present technology comprise a heavy chain having an amino acid sequence that is at least about 75%, 80%, 85%, 90%, 95%, 99%, or 100% identical to SEQ. ID NO: 1.

The heavy chain of the target antibodies may comprise a variable region domain (V_{H}) having an amino acid sequence that is about 75%, 80%, 85%, 90%, 95%, 99%, or 100% identical to SEQ. ID NO: 2.

The heavy chain of the target antibodies may comprise a V_{H} having an amino acid sequence that is at least 75%, 80%, 85%, 90%, 95%, 99%, or 100% identical to SEQ. ID NO: 2.

The heavy chain of the target antibodies may comprise a V_{H} having an amino acid sequence that is at least about 75%, 80%, 85%, 90%, 95%, 99%, or 100% identical to SEQ. ID NO: 2.

The heavy chain of the target antibodies may comprise a constant region having an amino acid sequence that is about 75%, 80%, 85%, 90%, 95%, 99%, or 100% identical to SEQ. ID NO: 3.

The heavy chain of the target antibodies may comprise a constant region having an amino acid sequence that is at least 75%, 80%, 85%, 90%, 95%, 99%, or 100% identical to SEQ. ID NO: 3.

The heavy chain of the target antibodies may comprise a constant region having an amino acid sequence that is at least about 75%, 80%, 85%, 90%, 95%, 99%, or 100% identical to SEQ. ID NO: 3.

The target antibodies of the present technology comprise a heavy chain having at least one Complementarity Determining Region (CDR) sequence. The at least one CDR sequence confers binding to a specific antigen (e.g., a TNFα peptide).

In some embodiments, the target antibodies comprise a heavy chain having a CDR having an amino acid sequence that is about 75%, 80%, 85%, 90%, 95%, 99%, or 100% identical to SEQ. ID NO: 9.

In some embodiments, the target antibodies comprise a heavy chain having a CDR having an amino acid sequence that is at least 75%, 80%, 85%, 90%, 95%, 99%, or 100% identical to SEQ. ID NO: 9.

In some embodiments, the target antibodies comprise a heavy chain having a CDR having an amino acid sequence that is at least about 75%, 80%, 85%, 90%, 95%, 99%, or 100% identical to SEQ. ID NO: 9.

In some embodiments, the target antibodies comprise a heavy chain having a CDR having an amino acid sequence that is about 75%, 80%, 85%, 90%, 95%, 99%, or 100% identical to SEQ. ID NO: 10.

In some embodiments, the target antibodies comprise a heavy chain having a CDR having an amino acid sequence that is at least 75%, 80%, 85%, 90%, 95%, 99%, or 100% identical to SEQ. ID NO: 10.

In some embodiments, the target antibodies comprise a heavy chain having a CDR having an amino acid sequence that is at least about 75%, 80%, 85%, 90%, 95%, 99%, or 100% identical to SEQ. ID NO: 10.

In some embodiments, the target antibodies comprise a heavy chain having a CDR having an amino acid sequence that is about 75%, 80%, 85%, 90%, 95%, 99%, or 100% identical to SEQ. ID NO: 11.

In some embodiments, the target antibodies comprise a heavy chain having a CDR having an amino acid sequence that is at least 75%, 80%, 85%, 90%, 95%, 99%, or 100% identical to SEQ. ID NO: 11.

In some embodiments, the target antibodies comprise a heavy chain having a CDR having an amino acid sequence that is at least about 75%, 80%, 85%, 90%, 95%, 99%, or 100% identical to SEQ. ID NO: 11.

In some embodiments, the target antibodies comprise a heavy chain having a CDR having an amino acid sequence that is about 75%, 80%, 85%, 90%, 95%, 99%, or 100% identical to SEQ. ID NO: 15.

In some embodiments, the target antibodies comprise a heavy chain having a CDR having an amino acid sequence that is at least 75%, 80%, 85%, 90%, 95%, 99%, or 100% identical to SEQ. ID NO: 15.

In some embodiments, the target antibodies comprise a heavy chain having a CDR having an amino acid sequence that is at least about 75%, 80%, 85%, 90%, 95%, 99%, or 100% identical to SEQ. ID NO: 15.

In some embodiments, the target antibodies comprise a heavy chain having a CDR having an amino acid sequence that is about 75%, 80%, 85%, 90%, 95%, 99%, or 100% identical to SEQ. ID NO: 16.

In some embodiments, the target antibodies comprise a heavy chain having a CDR having an amino acid sequence that is at least 75%, 80%, 85%, 90%, 95%, 99%, or 100% identical to SEQ. ID NO: 16.

In some embodiments, the target antibodies comprise a heavy chain having a CDR having an amino acid sequence that is at least about 75%, 80%, 85%, 90%, 95%, 99%, or 100% identical to SEQ. ID NO: 16.

In some embodiments, the target antibodies comprise a heavy chain having a CDR having an amino acid sequence that is about 75%, 80%, 85%, 90%, 95%, 99%, or 100% identical to SEQ. ID NO: 17.

In some embodiments, the target antibodies comprise a heavy chain having a CDR having an amino acid sequence that is at least 75%, 80%, 85%, 90%, 95%, 99%, or 100% identical to SEQ. ID NO: 17.

In some embodiments, the target antibodies comprise a heavy chain having a CDR having an amino acid sequence that is at least about 75%, 80%, 85%, 90%, 95%, 99%, or 100% identical to SEQ. ID NO: 17.

In some embodiments, the target antibodies comprise a heavy chain comprising (1) a CDR having an amino acid sequence that is about 75%, 80%, 85%, 90%, 95%, 99%, or 100% identical to SEQ. ID NO: 9, (2) a CDR having an amino acid sequence that is about 75%, 80%, 85%, 90%, 95%, 99%, or 100% identical to SEQ. ID NO: 10, and (3) a CDR having an amino acid sequence that is about 75%, 80%, 85%, 90%, 95%, 99%, or 100% identical to SEQ. ID NO: 11.

In some embodiments, the target antibodies comprise a heavy chain comprising (1) a CDR having an amino acid sequence that is at least 75%, 80%, 85%, 90%, 95%, 99%, or 100% identical to SEQ. ID NO: 9, (2) a CDR having an amino acid sequence that is at least 75%, 80%, 85%, 90%, 95%, 99%, or 100% identical to SEQ. ID NO: 10, and (3) a CDR having an amino acid sequence that is at least 75%, 80%, 85%, 90%, 95%, 99%, or 100% identical to SEQ. ID NO: 11.

In some embodiments, the target antibodies comprise a heavy chain comprising (1) a CDR having an amino acid sequence that is at least about 75%, 80%, 85%, 90%, 95%, 99%, or 100% identical to SEQ. ID NO: 9, (2) a CDR having an amino acid sequence that is at least about 75%, 80%, 85%, 90%, 95%, 99%, or 100% identical to SEQ. ID NO: 10, and (3) a CDR having an amino acid sequence that is at least about 75%, 80%, 85%, 90%, 95%, 99%, or 100% identical to SEQ. ID NO: 11.

In some embodiments, the target antibodies comprise a heavy chain comprising (1) a CDR having an amino acid sequence that is about 75%, 80%, 85%, 90%, 95%, 99%, or 100% identical to SEQ. ID NO: 15, (2) a CDR having an amino acid sequence that is about 75%, 80%, 85%, 90%, 95%, 99%, or 100% identical to SEQ. ID NO: 16, and (3) a CDR having an amino acid sequence that is about 75%, 80%, 85%, 90%, 95%, 99%, or 100% identical to SEQ. ID NO: 17.

In some embodiments, the target antibodies comprise a heavy chain comprising (1) a CDR having an amino acid sequence that is at least 75%, 80%, 85%, 90%, 95%, 99%, or 100% identical to SEQ. ID NO: 15, (2) a CDR having an amino acid sequence that is at least 75%, 80%, 85%, 90%, 95%, 99%, or 100% identical to SEQ. ID NO: 16, and (3) a CDR having an amino acid sequence that is at least 75%, 80%, 85%, 90%, 95%, 99%, or 100% identical to SEQ. ID NO: 17.

In some embodiments, the target antibodies comprise a heavy chain comprising (1) a CDR having an amino acid sequence that is at least about 75%, 80%, 85%, 90%, 95%, 99%, or 100% identical to SEQ. ID NO: 15, (2) a CDR having an amino acid sequence that is at least about 75%, 80%, 85%, 90%, 95%, 99%, or 100% identical to SEQ. ID NO: 16, and (3) a CDR having an amino acid sequence that is at least about 75%, 80%, 85%, 90%, 95%, 99%, or 100% identical to SEQ. ID NO: 17.

In some embodiments, the heavy chain of the target antibodies of the present technology is encoded by a nucleotide sequence that is about 75%, 80%, 85%, 90%, 95%, 99%, or 100% identical to SEQ. ID NO: 8.

In some embodiments, the heavy chain of the target antibodies of the present technology is encoded by a nucleotide sequence that is at least 75%, 80%, 85%, 90%, 95%, 99%, or 100% identical to SEQ. ID NO: 8.

In some embodiments, the heavy chain of the target antibodies of the present technology is encoded by a nucleotide sequence that is at least about 75%, 80%, 85%, 90%, 95%, 99%, or 100% identical to SEQ. ID NO: 8.

### Light Chain:

The target antibodies of the present technology comprise a light chain having an amino acid sequence that is about 75%, 80%, 85%, 90%, 95%, 99%, or 100% identical to SEQ. ID NO: 4.

The target antibodies of the present technology comprise a light chain having an amino acid sequence that is at least 75%, 80%, 85%, 90%, 95%, 99%, or 100% identical to SEQ. ID NO: 4.

The target antibodies of the present technology comprise a light chain having an amino acid sequence that is at least about 75%, 80%, 85%, 90%, 95%, 99%, or 100% identical to SEQ. ID NO: 4.

The light chain of the target antibodies may comprise a variable region domain (V_{L}) having an amino acid sequence that is about 75%, 80%, 85%, 90%, 95%, 99%, or 100% identical to SEQ. ID NO: 5.

The light chain of the target antibodies may comprise a V_{L} having an amino acid sequence that is at least 75%, 80%, 85%, 90%, 95%, 99%, or 100% identical to SEQ. ID NO: 5.

The light chain of the target antibodies may comprise a V_{L} having an amino acid sequence that is at least about 75%, 80%, 85%, 90%, 95%, 99%, or 100% identical to SEQ. ID NO: 5.

The light chain of the target antibodies may comprise a constant region having an amino acid sequence that is about 75%, 80%, 85%, 90%, 95%, 99%, or 100% identical to SEQ. ID NO: 6.

The light chain of the target antibodies may comprise a constant region having an amino acid sequence that is at least 75%, 80%, 85%, 90%, 95%, 99%, or 100% identical to SEQ. ID NO: 6.

The light chain of the target antibodies may comprise a constant region having an amino acid sequence that is at least about 75%, 80%, 85%, 90%, 95%, 99%, or 100% identical to SEQ. ID NO: 6.

The target antibodies of the present technology comprise a light chain having at least one CDR sequence. The at least one CDR sequence confers binding to a specific antigen (e.g., a TNFα peptide).

In some embodiments, the target antibodies comprise a light chain having a CDR having an amino acid sequence that is about 75%, 80%, 85%, 90%, 95%, 99%, or 100% identical to SEQ. ID NO: 12.

In some embodiments, the target antibodies comprise a light chain having a CDR having an amino acid sequence that is at least 75%, 80%, 85%, 90%, 95%, 99%, or 100% identical to SEQ. ID NO: 12.

In some embodiments, the target antibodies comprise a light chain having a CDR having an amino acid sequence that is at least about 75%, 80%, 85%, 90%, 95%, 99%, or 100% identical to SEQ. ID NO: 12.

In some embodiments, the target antibodies comprise a light chain having a CDR having an amino acid sequence that is about 75%, 80%, 85%, 90%, 95%, 99%, or 100% identical to SEQ. ID NO: 13.

In some embodiments, the target antibodies comprise a light chain having a CDR having an amino acid sequence that is at least 75%, 80%, 85%, 90%, 95%, 99%, or 100% identical to SEQ. ID NO: 13.

In some embodiments, the target antibodies comprise a light chain having a CDR having an amino acid sequence that is at least about 75%, 80%, 85%, 90%, 95%, 99%, or 100% identical to SEQ. ID NO: 13.

In some embodiments, the target antibodies comprise a light chain having a CDR having an amino acid sequence that is about 75%, 80%, 85%, 90%, 95%, 99%, or 100% identical to SEQ. ID NO: 14.

In some embodiments, the target antibodies comprise a light chain having a CDR having an amino acid sequence that is at least 75%, 80%, 85%, 90%, 95%, 99%, or 100% identical to SEQ. ID NO: 14.

In some embodiments, the target antibodies comprise a light chain having a CDR having an amino acid sequence that is at least about 75%, 80%, 85%, 90%, 95%, 99%, or 100% identical to SEQ. ID NO: 14.

In some embodiments, the target antibodies comprise a light chain having a CDR having an amino acid sequence that is about 75%, 80%, 85%, 90%, 95%, 99%, or 100% identical to SEQ. ID NO: 18.

In some embodiments, the target antibodies comprise a light chain having a CDR having an amino acid sequence that is at least 75%, 80%, 85%, 90%, 95%, 99%, or 100% identical to SEQ. ID NO: 18.

In some embodiments, the target antibodies comprise a light chain having a CDR having an amino acid sequence that is at least about 75%, 80%, 85%, 90%, 95%, 99%, or 100% identical to SEQ. ID NO: 18.

In some embodiments, the target antibodies comprise a light chain having a CDR having an amino acid sequence that is about 75%, 80%, 85%, 90%, 95%, 99%, or 100% identical to SEQ. ID NO: 19.

In some embodiments, the target antibodies comprise a light chain having a CDR having an amino acid sequence that is at least 75%, 80%, 85%, 90%, 95%, 99%, or 100% identical to SEQ. ID NO: 19.

In some embodiments, the target antibodies comprise a light chain having a CDR having an amino acid sequence that is at least about 75%, 80%, 85%, 90%, 95%, 99%, or 100% identical to SEQ. ID NO: 19.

In some embodiments, the target antibodies comprise a light chain having a CDR having an amino acid sequence that is about 75%, 80%, 85%, 90%, 95%, 99%, or 100% identical to SEQ. ID NO: 20.

In some embodiments, the target antibodies comprise a light chain having a CDR having an amino acid sequence that is at least 75%, 80%, 85%, 90%, 95%, 99%, or 100% identical to SEQ. ID NO: 20.

In some embodiments, the target antibodies comprise a light chain having a CDR having an amino acid sequence that is at least about 75%, 80%, 85%, 90%, 95%, 99%, or 100% identical to SEQ. ID NO: 20.

In some embodiments, the target antibodies comprise a light chain comprising (1) a CDR having an amino acid sequence that is about 75%, 80%, 85%, 90%, 95%, 99%, or 100% identical to SEQ. ID NO: 12, (2) a CDR having an amino acid sequence that is about 75%, 80%, 85%, 90%, 95%, 99%, or 100% identical to SEQ. ID NO: 13, and (3) a CDR having an amino acid sequence that is about 75%, 80%, 85%, 90%, 95%, 99%, or 100% identical to SEQ. ID NO: 14.

In some embodiments, the target antibodies comprise a light chain comprising (1) a CDR having an amino acid sequence that is at least 75%, 80%, 85%, 90%, 95%, 99%, or 100% identical to SEQ. ID NO: 12, (2) a CDR having an amino acid sequence that is at least 75%, 80%, 85%, 90%, 95%, 99%, or 100% identical to SEQ. ID NO: 13, and (3) a CDR having an amino acid sequence that is at least 75%, 80%, 85%, 90%, 95%, 99%, or 100% identical to SEQ. ID NO: 14.

In some embodiments, the target antibodies comprise a light chain comprising (1) a CDR having an amino acid sequence that is at least about 75%, 80%, 85%, 90%, 95%, 99%, or 100% identical to SEQ. ID NO: 12, (2) a CDR having an amino acid sequence that is at least about 75%, 80%, 85%, 90%, 95%, 99%, or 100% identical to SEQ. ID NO: 13, and (3) a CDR having an amino acid sequence that is at least about 75%, 80%, 85%, 90%, 95%, 99%, or 100% identical to SEQ. ID NO: 14.

In some embodiments, the target antibodies comprise a light chain comprising (1) a CDR having an amino acid sequence that is about 75%, 80%, 85%, 90%, 95%, 99%, or 100% identical to SEQ. ID NO: 18, (2) a CDR having an amino acid sequence that is about 75%, 80%, 85%, 90%, 95%, 99%, or 100% identical to SEQ. ID NO: 19, and (3) a CDR having an amino acid sequence that is about 75%, 80%, 85%, 90%, 95%, 99%, or 100% identical to SEQ. ID NO: 20.

In some embodiments, the target antibodies comprise a light chain comprising (1) a CDR having an amino acid sequence that is at least 75%, 80%, 85%, 90%, 95%, 99%, or 100% identical to SEQ. ID NO: 18, (2) a CDR having an amino acid sequence that is at least 75%, 80%, 85%, 90%, 95%, 99%, or 100% identical to SEQ. ID NO: 19, and (3) a CDR having an amino acid sequence that is at least 75%, 80%, 85%, 90%, 95%, 99%, or 100% identical to SEQ. ID NO: 20.

In some embodiments, the target antibodies comprise a light chain comprising (1) a CDR having an amino acid sequence that is at least about 75%, 80%, 85%, 90%, 95%, 99%, or 100% identical to SEQ. ID NO: 18, (2) a CDR having an amino acid sequence that is at least about 75%, 80%, 85%, 90%, 95%, 99%, or 100% identical to SEQ. ID NO: 19, and (3) a CDR having an amino acid sequence that is at least about 75%, 80%, 85%, 90%, 95%, 99%, or 100% identical to SEQ. ID NO: 20.

In some embodiments, the light chain of the target antibodies of the present technology is encoded by a nucleotide sequence that is about 75%, 80%, 85%, 90%, 95%, 99%, or 100% identical to SEQ. ID NO: 7.

In some embodiments, the light chain of the target antibodies of the present technology is encoded by a nucleotide sequence that is at least 75%, 80%, 85%, 90%, 95%, 99%, or 100% identical to SEQ. ID NO: 7.

In some embodiments, the light chain of the target antibodies of the present technology is encoded by a nucleotide sequence that is at least about 75%, 80%, 85%, 90%, 95%, 99%, or 100% identical to SEQ. ID NO: 7.

TABLE 1
Sequences of Example Anti-TNFα Antibodies of the Present Technology

| SEQ ID NO: | Description | Sequence |
|---|---|---|
| 1 | aa sequence of | EVKLEESGGGLVQPGGSMKLSCVASGFIFSNHWMNWVRQ |
| | heavy chain of | SPEKGLEWVAEIRSKSINSATHYAESVKGRFTISRDDSKSAV |
| | anti-TNFα | YLQMTDLRTEDTGVYYCSRNYYGSTYDYWGQGTTLTVSS |
| | antibody | ASTKGPSVFPLAPSSKSTSGGTAALGCLVKDYFPEPVTVSW |
| | (XTMAB-16, | NSGALTSGVHTFPAVLQSSGLYSLSSVVTVPSSSLGTQTYIC |
| | target antibody) | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| 2 | aa sequence of | EVKLEESGGGLVQPGGSMKLSCVASGFIFSNHWMNWVRQS |
| | heavy chain | PEKGLEWVAEIRSKSINSATHYAESVKGRFTISRDDSKSAV |
| | variable region | YLQMTDLRTEDTGVYYCSRNYYGSTYDYWGQGTTLTV |
| | of anti-TNFα | |
| | antibody | |
| | (XTMAB-16, | |
| | target antibody) | |
| 3 | aa sequence of | SSASTKGPSVFPLAPSSKSTSGGTAALGCLVKDYFPEPVTVS |
| | heavy chain | WNSGALTSGVHTFPAVLQSSGLYSLSSVVTVPSSSLGTQTYI |
| | constant region | CNVNHKPSNTKVDKKVEPKSCDKTHTCPPCPAPELLGGPS |
| | of anti-TNFα | VFLFPPKPKDTLMISRTPEVTCVVVDVSHEDPEVKENWYV |
| | antibody | DGVEVHNAKTKPREEQYNSTYRVVSVLTVLHQDWLNGKE |
| | (XTMAB-16, | YKCKVSNKALPAPIEKTISKAKGQPREPQVYTLPPSRDELTK |
| | target antibody) | NQVSLTCLVKGFYPSDIAVEWESNGQPENNYKTTPPVLDSD |
| | | GSFFLYSKLTVDKSRWQQGNVFSCSVMHEALHNHYTQKSL |
| | | SLSPGK |
| 4 | aa sequence of | DILLTQSPAILSVSPGERVSFSCRASQFVGSSIHWYQQRING |
| | light chain of | SPRLLIKYASESMSGIPSRFSGSGSGTDFTLSINTVESEDIAD |
| | anti-TNFα | YYCQQSHSWPFTFGSGTNLEVKRTVAAPSVFIFPPSDEQLK |
| | antibody | SGTASVVCLLNNFYPREAKVQWKVDNALQSGNSQESVTE |
| | (XTMAB-16, | QDSKDSTYSLSSTLTLSKADYEKHKVYACEVTHQGLSSPVT |
| | target antibody) | KSFNRGEC |
| 5 | aa sequence of | DILLTQSPAILSVSPGERVSFSCRASQFVGSSIHWYQQRTNG |
| | light chain | SPRLLIKYASESMSGIPSRFSGSGSGTDFTLSINTVESEDIAD |
| | variable region | YYCQQSHSWPFTFGSGTNLEV |
| | of anti-TNFα | |
| | antibody | |
| | (XTMAB-16, | |
| | target antibody) | |
| 6 | aa sequence of | KRTVAAPSVFIFPPSDEQLKSGTASVVCLLNNFYPREAKVQ |
| | light chain | WKVDNALQSGNSQESVTEQDSKDSTYSLSSTLTLSKADYE |
| | constant region | KHKVYACEVTHQGLSSPVTKSFNRGEC |
| | of anti-TNFα | |
| | antibody | |
| | (XTMAB-16, | |
| | target antibody) | |
| 7 | Nucleotide | GCTAGCGAATTCCACCATGGGCTGGTCCTGCATCATCCT |
| | sequence | GTTTCTGGTGGCCACCGCCACCGGCGTGCACTCCGACAT |
| | encoding of light | CCTGCTGACCCAGTCCCCCGCCATCCTGTCCGTGTCTCCC |
| | chain of anti- | GGCGAGCGGGTGTCCTTCTCCTGCCGGGCCTCCCAGTTC |
| | TNFα antibody | GTGGGCTCCTCCATCCACTGGTATCAGCAGCGGACCAAC |
| | (XTMAB-16, | GGCTCCCCTCGGCTGCTGATCAAGTACGCCTCCGAGTCC |
| | target antibody) | ATGTCCGGCATCCCTTCCCGGTTCTCCGGCTCCGGCTCTG |
| | | GCACCGACTTCACCCTGTCCATCAACACCGTGGAGTCCG |
| | | AGGATATTGCCGACTACTACTGCCAGCAGTCCCACTCCT |
| | | GGCCTTTCACCTTCGGCTCCGGCACCAACCTGGAGGTGA |
| | | AGCGTACGGTGGCTGCACCATCTGTCTTCATCTTCCCGCC |
| | | ATCTGATGAGCAGTTGAAATCTGGAACTGCCTCTGTTGT |
| | | GTGCCTGCTGAATAACTTCTATCCCAGAGAGGCCAAAGT |
| | | ACAGTGGAAGGTGGATAACGCCCTCCAATCGGGTAACTC |
| | | CCAGGAGAGTGTCACAGAGCAGGACAGCAAGGACAGCA |
| | | CCTACAGCCTCAGCAGCACCCTGACGCTGAGCAAAGCAG |
| | | ACTACGAGAAACACAAAGTCTACGCCTGCGAAGTCACCC |
| | | ATCAGGGCCTGAGCTCGCCCGTCACAAAGAGCTTCAACA |
| | | GGGGAGAGTGTTAA |
| 8 | Nucleotide | GCTAGCGATATCGAATTCTTTTCTTTTGTTTTGTTTCCACC |
| | sequence | ATGGGCTGGTCCTGCATCATCCTGTTTCTGGTGGCCACCG |
| | encoding of | CCACCGGCGTGCACTCCGAGGTGAAGCTGGAGGAGTCTG |
| | heavy chain of | GCGGCGGACTGGTGCAGCCTGGCGGCTCCATGAAGCTGT |
| | anti-TNFα | CCTGCGTGGCCTCCGGCTTCATCTTCTCCAACCACTGGAT |
| | antibody | GAACTGGGTGCGCCAGTCCCCTGAGAAGGGCCTGGAGT |
| | (XTMAB-16, | GGGTGGCCGAGATCCGGTCCAAGTCCATCAACTCCGCCA |
| | target antibody) | CCCACTACGCCGAGTCCGTGAAGGGCCGGTTCACCATCT |
| | | CCCGGGACGACTCCAAGTCCGCCGTGTACCTGCAGATGA |
| | | CCGACCTGCGGACCGAGGACACCGGAGTGTACTACTGCT |
| | | CCCGGAACTACTACGGCTCCACCTACGACTACTGGGGCC |
| | | AGGGCACCACACTGACCGTGAGCTCCGCCTCCACCAAGG |
| | | GCCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAG |
| | | GCGCCCTGACCAGCGGCGTGCACACCTTCCCGGCTGTCC |
| | | TACAGTCCTCAGGACTCTACTCCCTCAGCAGCGTGGTGA |
| | | CCGTGCCCTCCAGCAGCTTGGGCACCCAGACCTACATCT |
| | | GCAACGTGAATCACAAGCCCAGCAACACCAAGGTGGAC |
| | | AAGAAAGTCGAGCCCAAATCTTGTGACAAAACTCACAC |
| | | ATGCCCACCGTGCCCAGCACCTGAACTCCTGGGGGGACC |
| | | GTCAGTCTTCCTCTTCCCCCCAAAACCCAAGGACACCCT |
| | | CATGATCTCCCGGACCCCTGAGGTCACATGCGTGGTGGT |
| | | GGACGTGAGCCACGAAGACCCTGAGGTCAAGTTCAACT |
| | | GGTACGTGGACGGCGTGGAGGTGCATAATGCCAAGACA |
| | | AAGCCGCGGGAGGAGCAGTACAACAGCACGTACCGTGT |
| | | GGTCAGCGTCCTCACCGTCCTGCACCAGGACTGGCTGAA |
| | | TGGCAAGGAGTACAAGTGCAAGGTCTCCAACAAAGCCC |
| | | TCCCAGCCCCCATCGAGAAAACCATCTCCAAAGCCAAAG |
| | | GGCAGCCCCGAGAACCACAGGTGTACACCCTGCCCCCAT |
| | | CCCGGGATGAGCTGACCAAGAACCAGGTCAGCCTGACCT |
| | | GCCTGGTCAAAGGCTTCTATCCCAGCGACATCGCCGTGG |
| | | AGTGGGAGAGTAATGGGCAGCCGGAGAACAACTACAAG |
| | | ACCACGCCTCCCGTGCTGGACTCCGACGGCTCCTTCTTCC |
| | | TCTACAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCT |
| | | CCGGGTAAATAA |
| 9 | aa sequence of | GFIFSNHW |
| | heavy chain | |
| | CDR1 predicted | |
| | by CDR-IMGT | |
| | of anti-TNFα | |
| | antibody | |
| | (XTMAB-16, | |
| | target antibody) | |
| 10 | aa sequence of | IRSKSINSAT |
| | heavy chain | |
| | CDR2 predicted | |
| | by CDR-IMGT | |
| | of anti-TNFα | |
| | antibody | |
| | (XTMAB-16, | |
| | target antibody) | |
| 11 | aa sequence of | SRNYYGSTYDY |
| | heavy chain | |
| | CDR3 predicted | |
| | by CDR-IMGT | |
| | of anti-TNFα | |
| | antibody | |
| | (XTMAB-16, | |
| | target antibody) | |
| 12 | aa sequence of | QFVGSS |
| | light chain CDR1 | |
| | predicted by | |
| | CDR-IMGT of | |
| | anti-TNFα | |
| | antibody | |
| | (XTMAB-16, | |
| | target antibody) | |
| | aa sequence of | YA |
| | light chain CDR2 | |
| | predicted by | |
| | CDR-IMGT of | |
| | anti-TNFα | |
| | antibody | |
| | (XTMAB-16, | |
| | target antibody) | |
| 14 | aa sequence of | QQSHSWPFT |
| | light chain CDR3 | |
| | predicted by | |
| | CDR-IMGT of | |
| | anti-TNFα | |
| | antibody | |
| | (XTMAB-16, | |
| | target antibody) | |
| 15 | aa sequence of | NHWMN |
| | heavy chain | |
| | CDR1 predicted | |
| | by CDR-Kabat | |
| | of anti-TNFα | |
| | antibody | |
| | (XTMAB-16, | |
| | target antibody) | |
| 16 | aa sequence of | EIRSKSINSATHYAESVKG |
| | heavy chain | |
| | CDR2 predicted | |
| | by CDR-Kabat | |
| | of anti-TNFα | |
| | antibody | |
| | (XTMAB-16, | |
| | target antibody) | |
| 17 | aa sequence of | NYYGSTYDY |
| | heavy chain | |
| | CDR3 predicted | |
| | by CDR-Kabat | |
| | of anti-TNFα | |
| | antibody | |
| | (XTMAB-16, | |
| | target antibody) | |
| 18 | aa sequence of | RASQFVGSSIH |
| | light chain CDR1 | |
| | predicted by | |
| | CDR-Kabat of | |
| | anti-TNFα | |
| | antibody | |
| | (XTMAB-16, | |
| | target antibody) | |
| 19 | aa sequence of | YASESMS |
| | light chain CDR2 | |
| | predicted by | |
| | CDR-Kabat of | |
| | anti-TNFα | |
| | antibody | |
| | (XTMAB-16, | |
| | target antibody) | |
| 20 | aa sequence of | QQSHSWPFT |
| | light chain CDR3 | |
| | predicted by | |
| | CDR-Kabat of | |
| | anti-TNFα | |
| | antibody | |
| | (XTMAB-16, | |
| | target antibody) | |

### Antibody Labels

The target antibodies of the present technology may be labeled, such as with an affinity label or a detection label. Nonlimiting examples of antibody labels include a biotin label, a fluorescent label, an enzyme label (e.g., horseradish peroxidase (HRP) and alkaline phosphatase (AP)), gold nanoparticles, radioisotopes, and drug molecules.

In some embodiments, the target antibodies are conjugated to an additional molecule or compound, forming an antibody conjugate. The conjugation may occur via a covalent linkage.

In some embodiments, the target antibodies of the present technology are directly (e.g., labeled via conjugation) or indirectly labeled with an affinity label or a detection label. A detection label may be any label that may be detected directly or indirectly. As used herein, “direct” detection of the detection label includes measuring the presence of the detection label or inducing the detection label to produce a detectable substance. For example, the detection label may be a luminescent compound. In some embodiments, the detection is configured to produce a chemiluminescent or ECL signal in the presence of a marker. In some embodiments, the detection label is an enzyme configured to convert a chromogenic marker into a pigment. For example, the enzyme is a HRP and the chromogenic marker may be 3,3′,5,5′-tetramethylbenzidine (TMB). In some embodiments, the detection label comprises a sulfo-Tag. The sulfo-Tag (Ru-tag) may be contacted with a marker comprising tripropylamine (TPA), and the level of the detection label may be measured by applying an electrical current to the marker in the presence of the detection label to produce the ECL signal.

In some embodiments, the detection label is detected by a meso scale discovery (MSD) assay. In some embodiments, the detection label is detected by ECL. In some embodiments, the detection label is a ruthenium (Ru) metal ion. In some embodiments, the Ru ion is detected when the ion comes in close proximity with an electrode. As used herein, a “close proximity” between the Ru ion and the electrode is a distance between the Ru ion and the electrode that is small enough to cause a detectable oxidation-reduction reaction by ECL as understood by a person of skill in the art. In some embodiments, the solid phase comprises an electrode. In some embodiments, a Ru ion is detected by a charge-coupled device (CCD) camera when it is in close proximity with the electrode.

In some embodiments, the detection label is detected indirectly. As used herein, “indirect” detection of the detection antibody includes adding a separate component that interacts with the detection label, where the separate component is capable of being detected. For example, a secondary antibody may be added, where the secondary antibody is conjugated to a detectable luminescent probe or enzyme configured to convert a chromogenic substrate into a pigment (e.g., a detection antibody conjugated to a detection label).

An affinity label may be any molecular label that has affinity with the second affinity label. In some embodiments, a first affinity label is configured to make a covalent bond with the second affinity label. For example, the first affinity label may be biotin and the second affinity label may be streptavidin or vice versa. The first and second affinity labels may be any pairing of affinity labels known to one of ordinary skill in the art. In some embodiments, the first affinity label may be glutathione and the second affinity label may be glutathione S-transferase or vice versa. In some embodiments, the first affinity label may be maltose and the second affinity label may be maltose-binding protein or vice versa. In some embodiments, the first affinity label is chitin and the second affinity label is chitin-binding protein or vice versa.

In some embodiments, the detection label may be a third affinity label and detecting the detection label further comprises contacting the third affinity label with a fourth affinity label conjugated to a detectable compound, where the third affinity label binds to the fourth affinity label. For example, the fourth affinity label may be conjugated to a luminescent compound or enzyme configured to convert a chromogenic substrate into a pigment.

The third affinity label may be any molecular label that has affinity with the fourth affinity label. In some embodiments, the third affinity label is configured to make a covalent bond with the fourth affinity label. For example, the third affinity label may be biotin and the fourth affinity label may be streptavidin or vice versa. The third and fourth affinity labels may be any pairing of affinity labels known to one of ordinary skill in the art. In some embodiments, the third affinity label may be glutathione and the fourth affinity label may be glutathione S-transferase or vice versa. In some embodiments, the third affinity label may be maltose and the fourth affinity label may be maltose-binding protein or vice versa. In some embodiments, the third affinity label is chitin and the fourth affinity label is chitin-binding protein or vice versa.

### Compositions

The present technology comprises compositions having antibodies for detecting ADAs. In some embodiments, the compositions comprise one or more target antibodies that are bound by ADAs. The one or more target antibodies bound by ADAs may include an affinity antibody and/or a detection antibody. Both the affinity antibody and the detection antibody may be anti-TNFα antibodies and/or may comprise one or more of a heavy chain, a light chain, a variable region (e.g., V_{H} or V_{L}), a constant region (e.g., a heavy chain constant region or a light chain constant region), or a CDR of the present technology.

In some embodiments, the affinity antibody comprises an affinity label. The affinity antibody may be labeled with the affinity label directly or indirectly. Nonlimiting examples of affinity labels include fluorescent labels, enzyme labels (e.g., horseradish peroxidase and alkaline phosphatase), biotin labels, streptavidin labels, and radioisotopes. In some embodiments, the detection antibody is conjugated to a detection label. Nonlimiting examples of detection labels include a biotin label, a fluorescent label, an enzyme label (e.g., horseradish peroxidase and alkaline phosphatase), gold nanoparticles, and radioisotopes.

In some embodiments, the compositions further comprise a second affinity antibody. The second affinity antibody may bind an affinity label, such as an affinity label conjugated to the first affinity antibody described in supra. In some embodiments, the second affinity antibody binds the affinity label when the first affinity antibody and/or the detection antibody are bound by one or more ADAs. The second affinity antibody may be an unlabeled antibody. Nonlimiting examples of unlabeled antibodies include an antibody that is not conjugated to an affinity label or a detection label.

In some embodiments, the compositions comprise a single composition having a first affinity antibody of the present technology, a detection antibody of the present technology, and a second affinity antibody of the present technology. In some embodiments, the compositions comprise (1) a first composition having a first affinity antibody and a detection antibody and (2) a second composition having a second affinity antibody. In some embodiments, the compositions comprise (1) a first composition having a first affinity antibody, (2) a second composition having a detection antibody, and (3) a third composition having a second affinity antibody. In some embodiments, the compositions comprise (1) a first composition having a first affinity antibody and (2) a second composition having a detection antibody and a second affinity antibody. In some embodiments, the compositions comprise (1) a first composition having a first affinity antibody and a second affinity antibody and (2) a second composition having a detection antibody. In some embodiments, the first and second compositions are incubated with a sample simultaneously or sequentially. In some embodiments, the first and third compositions are incubated with a sample simultaneously or sequentially. In some embodiments, the second and third compositions are incubated with a sample simultaneously or sequentially. In some embodiments, the first, second, and third compositions are each incubated with a sample simultaneously or sequentially.

Samples may comprise biological samples. Nonlimiting examples of biological samples include serum, blood, synovial fluid, plasma, saliva, and respiratory secretions.

### Methods of Detecting ADAs

The present technology comprises methods for detecting the ADAs of the present technology in a sample. ADAs may be detected and/or isolated using the compositions of the present technology.

In some embodiments, detecting the ADA comprises incubating the sample with a composition of the present technology (e.g., a composition comprising one or more of an affinity antibody, a detection antibody, or an unlabeled antibody). In some embodiments, the affinity antibody is conjugated to the detection label.

In some embodiments, detecting the ADA comprises contacting the sample with a composition comprising a first affinity antibody of the present technology. In some embodiments, the affinity antibody is added at a concentration of about 1 ng/mL to about 10 ng/ml, about 10 ng/mL to about 20 ng/ml, about 20 ng/ml to about 50 ng/mL, or about 50 ng/mL about 100 ng/mL.

In some embodiments, the affinity antibody is added at a concentration of at least 1 ng/ml to at least 10 ng/mL, at least 10 ng/mL to at least 20 ng/ml, at least 20 ng/ml to at least 50 ng/mL, or at least 50 ng/ml at least 100 ng/ml.

In some embodiments, the affinity antibody is added at a concentration of at least about 1 ng/mL to at least about 10 ng/mL, at least about 10 ng/ml to at least about 20 ng/ml, at least about 20 ng/mL to at least about 50 ng/mL, or at least about 50 ng/mL at least about 100 ng/ml.

In some embodiments, the affinity antibody is added at a concentration of about 25 ng/mL.

In some embodiments, the affinity antibody is added at a concentration of at least 25 ng/mL.

In some embodiments, the affinity antibody is added at a concentration of at least about 25 ng/mL.

In some embodiments, detecting the ADA comprises incubating the sample with a composition comprising a detection antibody.

In some embodiments, the detection antibody is added at a concentration of about 0.001 mg/mL to about 0.01 mg/mL, about 0.01 mg/mL to about 0.1 mg/mL, about 0.1 mg/mL to about 1 mg/mL, about 1 mg/mL to about 10 mg/mL. In some embodiments, the affinity antibody is added at a concentration of about 1.2 mg/mL. In some embodiments, the detection antibody comprises a ruthenium label (i.e., Ru-XTMAB-16), and it is added at a concentration of about 1.2 mg/mL.

In some embodiments, the detection antibody is added at a concentration of at least 0.001 mg/mL to at least 0.01 mg/mL, at least 0.01 mg/mL to at least 0.1 mg/mL, at least 0.1 mg/mL to at least 1 mg/mL, at least 1 mg/mL to at least 10 mg/mL. In some embodiments, the affinity antibody is added at a concentration of at least 1.2 mg/mL. In some embodiments, the detection antibody comprises a ruthenium label (i.e., Ru-XTMAB-16), and it is added at a concentration of at least 1.2 mg/mL.

In some embodiments, the detection antibody is added at a concentration of at least about 0.001 mg/mL to at least about 0.01 mg/mL, at least about 0.01 mg/mL to at least about 0.1 mg/mL, at least about 0.1 mg/mL to at least about 1 mg/mL, at least about 1 mg/mL to at least about 10 mg/mL. In some embodiments, the affinity antibody is added at a concentration of at least about 1.2 mg/mL. In some embodiments, the detection antibody comprises a ruthenium label (i.e., Ru-XTMAB-16), and it is added at a concentration of at least about 1.2 mg/mL.

In some embodiments, the methods further comprise isolating an ADA that binds a target antibody of the present technology (e.g., an affinity antibody or a detection antibody). As used herein, “isolating” ADAs that form a complex with both the affinity antibody and the detection antibody includes identifying the ADAs that form a complex and separating them from ADAs that do not form a complex. Isolating ADAs also includes identifying and separating ADAs from other compounds that bind the target antibody. Isolating ADAs may be performed using any technique known to one of skill for isolating or purifying an antibody. For example, the ADAs may be isolated using antibody pull-down or purification techniques. In some embodiments, the ADAs are isolated by contacting the ADA complex with a solid phase comprising a second affinity label, such that the first affinity label of the ADA complex binds to the second affinity label on a solid phase.

As used herein, “solid phase” may be any medium that remains stationary or temporarily stationary as a mobile phase moves across or through the solid phase, such that components of the mobile phase may interact with the solid phase. A solid phase may include a plate, chromatography column, microbead, magnetic microbead, or any other solid phase known to one of ordinary skill in the art. In some embodiments, the solid phase is a microbead, plate, or column. In some embodiments, the solid phase is a plate. In some embodiments, the plate is coated with a second affinity label. In some embodiments, the solid phase is blocked with a blocking buffer before the sample and the composition are contacted with the solid phase.

In some embodiments, isolating the ADA complex comprises: (a) providing a solid phase comprising the second affinity label; (b) contacting the sample and the composition with the solid phase; and (c) washing the solid phase. In some embodiments, the solid phase is washed with a wash buffer that does not disrupt the interaction between the ADA and the affinity antibody or the interaction between the ADA and the detection antibody. In some embodiments, the wash buffer comprises phosphate-buffered saline (PBS). In some embodiments, the wash buffer comprises a phosphate-buffered solution with a surfactant. In some embodiments, the wash buffer comprises Tween-20. In some embodiments, the wash buffer comprises 1×PBS with 0.05% Tween-20. In some embodiments, the wash buffer comprises 50 mM Tris. In some embodiments, the wash buffer comprises 0.14 M NaCl. In some embodiments, the wash buffer has a pH of about 8.0. In some embodiments, the wash buffer comprises 50 mM Tris, 0.14 M NaCl, 0.05% Tween-20, and has a pH of about 8.0.

In some embodiments, the method further comprises detecting the detection label. In some embodiments, the detection label is detected by measuring a luminescent compound. In some embodiments, the detection label is detected by inducing an enzyme to convert a chromogenic substrate into the pigment and detecting the pigment and measuring the presence of the pigment. In some embodiments, detecting the detection label comprises converting the chromogenic substrate into the pigment and detecting the optical density of the sample. In some embodiments, detecting the detection label comprises contacting a sulfo-Tag (Ru-tag) detection label with a marker comprising TPA) and the level of the detection label may be detected by applying an electrical current to the marker in the presence of the detection label to produce a detectable ECL signal. As such, a higher level of the detection label correlates with a higher amount of ADAs in the sample.

In some embodiments, the ADA is detected by determining whether the detection label is present at a level above a predetermined level. In some embodiments, the ADA is detected by determining that the ECL of the sample is above a predetermined cut-point.

In some embodiments, the method further comprises performing the method on a negative control sample. In some embodiments, the negative control sample has no detectable ADA. In some embodiments, the negative control sample is a drug naïve sample from a healthy subject who has not received the target antibody. In some embodiments, the method is performed on the negative control sample in parallel with the sample. As used herein, performing the method “in parallel” includes performing the method on the negative control sample at the same time and/or on the same plate as the sample. Performing the method on the negative control sample comprises: (i) incubating the control sample with a composition comprising an affinity antibody and a detection antibody; (ii) contacting the complex with a second affinity label that binds to the first affinity label; and (iii) measuring a level of the detection label.

In some embodiments, the method further comprises performing the method on a positive control sample. In some embodiments, the positive control sample is a sample from a subject who has received the target antibody. In some embodiments, the positive control sample is a sample that has been shown to contain an ADA in a previous assay. The method may be performed on the positive control sample in parallel with the sample. The method may be performed on a sample, a negative control sample, and a positive control sample in parallel.

In some embodiments, the cut-point is determined by performing the method on a plurality of negative control samples. In some embodiments, the negative control samples are drug-naïve samples from healthy subjects who have never received the target antibody. In some embodiments, the cut-point is determined by performing the method on at least 20, 30, 40, or 50 negative control samples. In some embodiments, the ECL signal from the 50 negative control samples is normalized by the mean of all negative control samples. In some embodiments, outliers are identified and removed.

In some embodiments, the cut-point is identified based on about a 0.01%-0.05%, 0.05%-0.1%, 0.1%-0.5%, 0.5%-1.0%, 1%-2%, 2%-5%, or 5%-10% false-positive rate. In some embodiments, the cut-point is identified based on about a 0.1%, 1%, or 5% false-positive rate. In some embodiments, the cut-point is identified based on about a 5% false-positive rate. In some embodiments, the cut-point is identified based on about a 1% false-positive rate. In some embodiments, the cut-point is identified based on about a 0.1% false-positive rate.

In some embodiments, the cut-point is identified based on at least a 0.01%-0.05%, 0.05%-0.1%, 0.1%-0.5%, 0.5%-1.0%, 1%-2%, 2%-5%, or 5%-10% false-positive rate. In some embodiments, the cut-point is identified based on at least a 0.1%, 1%, or 5% false-positive rate. In some embodiments, the cut-point is identified based on at least a 5% false-positive rate. In some embodiments, the cut-point is identified based on at least a 1% false-positive rate. In some embodiments, the cut-point is identified based on at least a 0.1% false-positive rate.

In some embodiments, the cut-point is identified based on at least about a 0.01%-0.05%, 0.05%-0.1%, 0.1%-0.5%, 0.5%-1.0%, 1%-2%, 2%-5%, or 5%-10% false-positive rate. In some embodiments, the cut-point is identified based on at least about a 0.1%, 1%, or 5% false-positive rate. In some embodiments, the cut-point is identified based on at least about a 5% false-positive rate. In some embodiments, the cut-point is identified based on at least about a 1% false-positive rate. In some embodiments, the cut-point is identified based on at least about a 0.1% false-positive rate.

In some embodiments, the method further comprises performing the method on a quality control (QC) sample. One or more QC samples may be included to assess the performance of the method. In some embodiments, the QC sample is a blank sample, matrix spike sample (i.e., a plasma or serum sample spiked with ADA), field duplicate sample, replicate sample, and/or reference material sample. The method may be performed on the QC sample in parallel with the sample. The method may be performed on a sample, a negative control sample, a positive control sample, and a QC sample in parallel.

In some embodiments, the predetermined cut-point is an electrochemiluminescence (ECL) of about 0.1-1, 1-2, 2-5, or 5-10. In some embodiments, the predetermined cut-point is about 1. In some embodiments, the predetermined cut-point is about 1.1.

In some embodiments, the predetermined cut-point is an ECL of at least 0.1-1, 1-2, 2-5, or 5-10. In some embodiments, the predetermined cut-point is at least 1. In some embodiments, the predetermined cut-point is at least 1.1.

In some embodiments, the predetermined cut-point is an ECL of at least about 0.1-1, 1-2, 2-5, or 5-10. In some embodiments, the predetermined cut-point is at least about 1. In some embodiments, the predetermined cut-point is at least about 1.1.

In some embodiments, the sensitivity of detecting the ADA is less than about 3 ng/mL, less than about 5 ng/ml, less than about 10 ng/ml, less than about 50 ng/ml, or less than about 100 ng/mL. In some embodiments, the sensitivity of detecting the ADA is less than about 3 ng/ml. In some embodiments, the sensitivity of detecting the ADA is less than about 2.77 ng/ml. In some embodiments, the sensitivity of detecting the ADA is about 4 ng/mL. In some embodiments, the sensitivity of detecting the ADA is about 6 ng/ml.

In some embodiments, the sensitivity of detecting the ADA is less than at least 3 ng/ml, less than at least 5 ng/mL, less than at least 10 ng/ml, less than at least 50 ng/mL, or less than at least 100 ng/mL. In some embodiments, the sensitivity of detecting the ADA is less than at least 3 ng/mL. In some embodiments, the sensitivity of detecting the ADA is less than at least 2.77 ng/mL. In some embodiments, the sensitivity of detecting the ADA is at least 4 ng/ml. In some embodiments, the sensitivity of detecting the ADA is at least 6 ng/mL.

In some embodiments, the sensitivity of detecting the ADA is less than at least about 3 ng/mL, less than at least about 5 ng/ml, less than at least about 10 ng/ml, less than at least about 50 ng/mL, or less than at least about 100 ng/mL. In some embodiments, the sensitivity of detecting the ADA is less than at least about 3 ng/mL. In some embodiments, the sensitivity of detecting the ADA is less than at least about 2.77 ng/mL. In some embodiments, the sensitivity of detecting the ADA is at least about 4 ng/mL. In some embodiments, the sensitivity of detecting the ADA is at least about 6 ng/mL.

In some embodiments, the drug tolerance of detecting the ADA is about 1 ng/mL to about 10 ng/ml, about 10 ng/ml to about 50 ng/mL, about 50 ng/ml to about 100 ng/mL, about 100 ng/mL to about 1000 ng/mL, or about 1000 ng/mL to about 5000 ng/mL. In some embodiments, the drug tolerance is about 100 ng/ml.

In some embodiments, the drug tolerance of detecting the ADA is at least 1 ng/ml to at least 10 ng/mL, at least 10 ng/ml to at least 50 ng/mL, at least 50 ng/ml to at least 100 ng/ml, at least 100 ng/ml to at least 1000 ng/mL, or at least 1000 ng/mL to at least 5000 ng/mL. In some embodiments, the drug tolerance is at least 100 ng/ml.

In some embodiments, the drug tolerance of detecting the ADA is at least about 1 ng/ml to at least about 10 ng/mL, at least about 10 ng/mL to at least about 50 ng/ml, at least about 50 ng/mL to at least about 100 ng/mL, at least about 100 ng/ml to at least about 1000 ng/mL, or at least about 1000 ng/mL to at least about 5000 ng/mL. In some embodiments, the drug tolerance is at least about 100 ng/mL.

In some embodiments, the method comprises disassociating the ADA from the target by adding an acid wash to the sample. The acid wash may be any acid wash known to one of skill in the art to disassociate an antibody from its target. In some embodiments, the acid wash may comprise acetic acid. In some embodiments, the acid wash comprises about 2M acetic acid. In some embodiments, the acid wash is added to the sample before an anti-TNFα conjugate is added. In some embodiments, the acid wash comprises about 300 mM acetic acid. In some embodiments, the acid wash comprises about 0.1M glycine-HCl. In some embodiments, the acid has a pH of about 2.5.

In some embodiments, all or a portion of the method is performed using an automated system. As used herein, an “automated system” refers to a system that processes samples according to steps that are in part performed by a robotic or mechanical platform. The automated system may be completely automated or partially automated. In some embodiments, a human “loads” the automated system with the samples and reagents of the present technology and the automated system performs the remaining steps. For example, the automated system may be a KingFisher™ Flex Purification System. Other automated systems having at least the foregoing features and known to one of skill in the art are within the scope of this technology.

In some embodiments, the method for detecting an ADA that binds the target antibody in a sample comprises: (i) incubating the sample with a composition comprising: (a) an affinity antibody comprising a first affinity label and (b) a detection antibody comprising a detection label; (ii) providing a solid phase comprising a second affinity label, wherein the second affinity label is configured to bind to the first affinity label; (iii) contacting the sample and the composition with the solid phase; (iv) washing the solid phase; and (v) detecting the ADA by measuring a level of the detection label above a predetermined cut-point.

In some embodiments, the method for detecting an ADA that binds the target antibody in a sample comprises: (i) incubating the sample with a composition comprising an affinity antibody comprising a first affinity label; (ii) incubating the sample with a detection antibody comprising a detection label; (iii) providing a solid phase comprising a second affinity label, wherein the second affinity label is configured to bind to the first affinity label; (iv) contacting the sample and the composition with the solid phase; (v) washing the solid phase; and (vi) detecting the ADA by measuring a level of the detection label above a predetermined cut-point.

In some embodiments, the method for detecting an ADA that binds the target antibody in a sample comprises: (i) contacting a sample with an affinity antibody comprising a first affinity label and a detection antibody comprising a detection label; and (ii) measuring the ADA that forms a complex with the affinity antibody and the detection antibody by detecting the level of the detection label in the complex; wherein the method detects the ADA with a sensitivity less than 5 ng/ml.

The present technology also includes a method for estimating a quantity of an ADA that binds a target antibody in a sample. The method comprises: (i) incubating the sample with a composition comprising an affinity antibody and a detection antibody; (ii) isolating ADAs that form a complex with both the affinity antibody and the detection antibody, wherein the ADA is isolated by contacting the complex with a second affinity label that binds to the first affinity label; (iii) detecting the ADA by measuring a level of the detection label above a predetermined cut-point; (iv) performing steps (i)-(iii) on a known sample having a known concentration of target antibody; and (v) comparing the level of the detection label in the sample to the level of the detection label in the known sample. As used herein, estimating “a quantity” of an ADA includes estimating a concentration of an ADA. As used herein, a “titration assay” generally refers to a method for estimating a quantity of an ADA that binds a target antibody.

As used herein, a “known sample” includes a positive control where the concentration of ADAs has been previously determined. In some embodiments, the known sample is serially diluted into a plurality of titer samples. As used herein, a “titer sample” is one of a plurality of titer samples that are diluted from a known sample. As such, the titer samples create a dilution curve.

In some embodiments, the titer samples have a concentration of, for example, between about 0.49 ng/ml and about 4000 ng/mL. In some embodiments, the titer samples have a concentration of, for example, about 4000 ng/mL, about 2000 ng/mL, about 1000 ng/mL, about 500 ng/ml, about 250 ng/ml, about 125 ng/ml, about 62.5 ng/mL, about 31.25 ng/mL, about 15.63 ng/ml, about 7.81 ng/mL, about 3.91 ng/mL, about 1.95 ng/ml, about 0.98 ng/ml, and about 0.49 ng/mL.

In some embodiments, the titer samples have a concentration of, for example, between at least 0.49 ng/ml and at least 4000 ng/mL. In some embodiments, the titer samples have a concentration of, for example, at least 4000 ng/ml, at least 2000 ng/ml, at least 1000 ng/ml, at least 500 ng/mL, at least 250 ng/mL, at least 125 ng/mL, at least 62.5 ng/ml, at least 31.25 ng/ml, at least 15.63 ng/ml, at least 7.81 ng/ml, at least 3.91 ng/mL, at least 1.95 ng/mL, at least 0.98 ng/mL, and at least 0.49 ng/mL.

In some embodiments, the titer samples have a concentration of, for example, between at least about 0.49 ng/ml and at least about 4000 ng/mL. In some embodiments, the titer samples have a concentration of, for example, at least about 4000 ng/ml, at least about 2000 ng/ml, at least about 1000 ng/ml, at least about 500 ng/ml, at least about 250 ng/mL, at least about 125 ng/mL, at least about 62.5 ng/ml, at least about 31.25 ng/ml, at least about 15.63 ng/ml, at least about 7.81 ng/ml, at least about 3.91 ng/ml, at least about 1.95 ng/ml, at least about 0.98 ng/mL, and at least about 0.49 ng/ml.

In some embodiments, the quantity or concentration of the sample is estimated by comparing the level of the detection label in the sample to the level of the detection label in the known sample or the titer samples. In some embodiments, the method further comprises generating a curve of the levels of the detection label in each of the titer samples and comparing the detection label in the sample to the curve.

The present technology also includes a method for estimating a quantity of an ADA that binds a target antibody in a sample. The method comprises: (i) incubating the sample with a composition comprising an affinity antibody comprising a first affinity label and a detection antibody comprising a detection label; (ii) providing a solid phase comprising a second affinity label, wherein the second affinity label is configured to bind to the first affinity label; (iii) contacting the sample and the composition with the solid phase; (iv) washing the solid phase; (v) detecting the ADA by measuring a level of the detection label above a predetermined cut-point; (vi) performing steps (i)-(v) on a known sample having a known concentration of target antibody; and (vii) comparing the level of the detection label in the sample to the level of the detection label in the known sample. Step (vi) may be performed in parallel with steps (i)-(v).

The present technology also includes a method of confirming that an ADA compound binds a target antibody in a sample by performing a competitive binding assay comprising performing any of the methods described in this technology for detecting an ADA within the sample, wherein the composition further comprises unlabeled anti-TNFα antibody (unlabeled antibody). As used herein, a “confirmation assay” or “confirmatory assay” generally refers to a method of confirming that an ADA component binds a target antibody.

In some embodiments, the method of confirming that an ADA compound binds a target antibody comprises: (i) incubating the sample with a composition comprising: an affinity antibody, a detection antibody, and an unlabeled antibody; (ii) isolating ADAs that form a complex with both the affinity antibody and the detection antibody and/or unlabeled antibody, wherein the ADA is isolated by contacting the complex with a second affinity label that binds to the first affinity label; and (iii) confirming the ADA by measuring a level of the detection label that is lower by a predetermined factor than the level of the detection label when the unlabeled antibody is not present.

In some embodiments, the unlabeled antibody will be present at an excess concentration in the composition. For example, the excess concentration of the unlabeled antibody may be about 150 μg/mL or more, about 175 μg/mL or more, about 200 μg/mL or more, or about 250 μg/mL or more. In some embodiments, the excess concentration of the unlabeled antibody may be about 200 μg/mL or more.

In some embodiments, the excess concentration of the unlabeled antibody may be at least 150 μg/mL or more, at least 175 μg/mL or more, at least 200 μg/mL or more, or at least 250 μg/mL or more. In some embodiments, the excess concentration of the unlabeled antibody may be at least 200 μg/mL or more.

In some embodiments, the excess concentration of the unlabeled antibody may be at least about 150 μg/mL or more, at least about 175 μg/mL or more, at least about 200 μg/mL or more, or at least about 250 μg/mL or more. In some embodiments, the excess concentration of the unlabeled antibody may be at least about 200 μg/mL or more.

In some embodiments, ADA is confirmed in a sample by measuring a level of the detection label that is lower by a predetermined factor of about 10% or more, about 12% or more, about 14% or more, about 16% or more, about 18% or more, or about 20% or more than the level of the detection label when the unlabeled antibody is not present. In some embodiments, the predetermined factor is about 16% or more.

In some embodiments, ADA is confirmed in a sample by measuring a level of the detection label that is lower by a predetermined factor of at least 10% or more, at least 12% or more, at least 14% or more, at least 16% or more, at least 18% or more, or at least 20% or more than the level of the detection label when the unlabeled antibody is not present. In some embodiments, the predetermined factor is at least 16% or more.

In some embodiments, ADA is confirmed in a sample by measuring a level of the detection label that is lower by a predetermined factor of at least about 10% or more, at least about 12% or more, at least about 14% or more, at least about 16% or more, at least about 18% or more, or at least about 20% or more than the level of the detection label when the unlabeled antibody is not present. In some embodiments, the predetermined factor is at least about 16% or more.

In some embodiments, the level of the detection label when the unlabeled antibody is not present is above the pre-determined cut-point.

In some embodiments, the method of confirming that an ADA compound binds a target antibody comprises: (i) incubating the sample with a composition comprising: an affinity antibody, a detection antibody, and an unlabeled antibody; (ii) providing a solid phase comprising a second affinity label, wherein the second affinity label is configured to bind to the first affinity label; (iii) contacting the sample and the composition with the solid phase; (iv) washing the solid phase; and (v) confirming the ADA by measuring a level of the detection label that is lower by a predetermined factor than the level of the detection label when the unlabeled antibody is not present.

The methods of the present technology may further comprises steps for assessing one or more of hook effects, assay selectivity, drug tolerance, intra-assay precision, inter-assay precision, and sample, control, and/or antibody stability.

The hook (prozone) effect is a phenomenon in which the signal of the assay changes from increasing to decreasing when the ADA concentration increases. The hook effect may not be critical to ADA detection if all concentrations of positive antibody yield positive results.

Stability assessments may comprise quantifying the ability of the methods to detect ADAs in the presence of other matrix components.

Drug tolerance assessments may comprise evaluating the ability of the methods to correctly detect ADAs. The presence of drug antibodies in samples may interfere with the detection of ADAs.

Stability assessments may comprise assessing positive controls. Positive controls may be assessed, for example, in a biological sample such as human scrum. The ADAs of the present technology may be stable for up to six freeze/thaw cycles from about −70° C. (nominal) to ambient temperature and may be stable for up to about 24.5 hours at ambient surroundings.

### Kits

The present technology includes a kit for detecting an ADA against the target antibody. In some embodiments, the technology includes a kit for estimating the quantity of ADA in a sample. In some embodiments, the technology includes a kit for confirming that an ADA binds the target antibody.

In some embodiments, the kit comprises an affinity antibody comprising a first affinity label, a detection antibody, and a solid phase comprising a second affinity label. In some embodiments, the kit comprises a composition comprising the affinity antibody and the detection antibody.

In some embodiments, the solid phase is a plate. In some embodiments, the second affinity label is streptavidin. In some embodiments, the first affinity label is biotin. In some embodiments, the detection antibody comprises a sulfo-Tag labeled anti-TNFα antibody. In some embodiments, the kit further comprises a marker configured to produce a chemiluminescent or ECL signal in the presence of the detection label. In some embodiments, the marker is TPA.

In some embodiments, the kit includes an undetected antibody. In some embodiments, the undetected antibody is at an excess concentration, for example, about 200 μg/mL or more. The kit may also comprise a composition comprising an affinity antibody, a detection antibody, and an unlabeled antibody.

In some embodiments, the affinity antibody comprises a first affinity label. In some embodiments, the first affinity label is biotin. In some embodiments, the detection antibody comprises a detection label. In some embodiments, the detection label is a luminescent compound, an enzyme configured to convert a chromogenic substrate into a pigment, or biotin. In some embodiments, the detection label is biotin.

In some embodiments, the kit comprises a negative control. The negative control may be a sample from a drug naïve subject. In some embodiments, the negative control is a sample that is known to contain no ADA. In some embodiments, the kit comprises a positive control. The positive control may be a sample from a subject that has received the target antibody. In some embodiments, the positive control is a sample known to contain ADA. In some embodiments, the kit may comprise ADA or ADA solution and instructions for making a positive control. For example, the kit may comprise an ADA solution and instructions to “spike” a plasma or serum sample with the ADA solution to prepare the positive control.

In some embodiments, the kit comprises a “high” positive control (HPC) and a “low” positive control (LPC), where the HPC has a higher concentration than the LPC. For example, the HPC may have a concentration of about 500 ng/mL to about 1000 ng/mL, about 1000 ng/ml to about 2000 ng/mL, about 2000 ng/mL to about 5000 ng/ml, or about 5000 ng/mL to about 10000 ng/ml. In some embodiments, the HPC has an ADA concentration of about 4000 ng/mL. The concentration of the LPC may be less than about 1 ng/mL, about 1 ng/mL to about 10 ng/ml, about 10 ng/ml to about 100 ng/mL, about 100 ng/ml to about 200 ng/mL, about 200 ng/ml to about 300 ng/ml, or about 300 ng/mL to about 500 ng/mL. In some embodiments, the LPC has an ADA concentration of about 9.7 ng/mL.

In some embodiments, the HPC may have a concentration of at least 500 ng/ml to at least 1000 ng/ml, at least 1000 ng/ml to at least 2000 ng/ml, at least 2000 ng/ml to at least 5000 ng/ml, or at least 5000 ng/mL to at least 10000 ng/ml. In some embodiments, the HPC has an ADA concentration of at least 4000 ng/mL. The concentration of the LPC may be less than at least 1 ng/mL, at least 1 ng/mL to at least 10 ng/mL, at least 10 ng/ml to at least 100 ng/ml, at least 100 ng/ml to at least 200 ng/mL, at least 200 ng/mL to at least 300 ng/mL, or at least 300 ng/ml to at least 500 ng/mL. In some embodiments, the LPC has an ADA concentration of at least 9.7 ng/mL.

In some embodiments, the HPC may have a concentration of at least about 500 ng/ml to at least about 1000 ng/ml, at least about 1000 ng/ml to at least about 2000 ng/ml, at least about 2000 ng/mL to at least about 5000 ng/ml, or at least about 5000 ng/ml to at least about 10000 ng/mL. In some embodiments, the HPC has an ADA concentration of at least about 4000 ng/ml. The concentration of the LPC may be less than at least about 1 ng/mL, at least about 1 ng/ml to at least about 10 ng/mL, at least about 10 ng/mL to at least about 100 ng/ml, at least about 100 ng/mL to at least about 200 ng/mL, at least about 200 ng/ml to at least about 300 ng/mL, or at least about 300 ng/mL to at least about 500 ng/mL. In some embodiments, the LPC has an ADA concentration of at least about 9.7 ng/mL.

In some embodiments, the kit comprises a known sample. In some embodiments, the known sample has a concentration of ADAs of about 4000 ng/ml or more, about 40 μg/mL or more, about 400 μg/mL or more, or about 4000 μg/mL or more. The known sample may have a concentration of ADAs of about 4000 ng/mL or more. The kit may also comprise one or more titer samples. For example, the kit may comprise a titer sample having an ADA concentration of about 4000 ng/ml, about 2000 ng/ml, about 1000 ng/mL, about 500 ng/mL, about 250 ng/ml, about 125 ng/mL, about 62.5 ng/mL, about 31.25 ng/ml, about 15.63 ng/ml, about 7.81 ng/ml, about 3.91 ng/ml, about 1.95 ng/mL, about 0.98 ng/mL, and about 0.49 ng/mL.

In some embodiments, the kit comprises a known sample. In some embodiments, the known sample has a concentration of ADAs of at least 4000 ng/mL or more, at least 40 μg/mL or more, at least 400 μg/mL or more, or at least 4000 μg/mL or more. The known sample may have a concentration of ADAs of at least 4000 ng/ml or more. The kit may also comprise one or more titer samples. For example, the kit may comprise a titer sample having an ADA concentration of at least 4000 ng/ml, at least 2000 ng/ml, at least 1000 ng/ml, at least 500 ng/ml, at least 250 ng/ml, at least 125 ng/ml, at least 62.5 ng/ml, at least 31.25 ng/mL, at least 15.63 ng/ml, at least 7.81 ng/mL, at least 3.91 ng/mL, at least 1.95 ng/ml, at least 0.98 ng/ml, and at least 0.49 ng/ml.

In some embodiments, the kit comprises a known sample. In some embodiments, the known sample has a concentration of ADAs of at least about 4000 ng/ml or more, at least about 40 μg/mL or more, at least about 400 μg/mL or more, or at least about 4000 μg/mL or more. The known sample may have a concentration of ADAs of at least about 4000 ng/ml or more. The kit may also comprise one or more titer samples. For example, the kit may comprise a titer sample having an ADA concentration of at least about 4000 ng/ml, at least about 2000 ng/mL, at least about 1000 ng/ml, at least about 500 ng/mL, at least about 250 ng/ml, at least about 125 ng/ml, at least about 62.5 ng/ml, at least about 31.25 ng/mL, at least about 15.63 ng/ml, at least about 7.81 ng/ml, at least about 3.91 ng/mL, at least about 1.95 ng/ml, at least about 0.98 ng/mL, and at least about 0.49 ng/ml.

The affinity antibody and/or the detection antibody may be affinity purified. In some embodiments, the affinity antibody and/or the detection antibody may be stored as a lyophilized powder. In some embodiments, the affinity antibody and/or detection antibody may be stored as a reconstituted liquid antibody.

Any of the components of the kit may be provided at a concentration that is more concentrated than the concentration at which it is used. For example, the components of the kit may be provided as a “stock solution” that must be diluted to the desired concentration of the “working solution.” In some embodiments, the stock solution may be about 2×, 5×, 10×, 50×, 100×, 500×, 1000×, 5000×, or 10000× more concentrated than the working solution.

The affinity antibody and/or the detection antibody may be lyophilized using methods known to one of skill in the art. One or more lyophilizers may be included. Examples of suitable lyophilizers include, for example, Tris-EDTA and sucrose. Any other lyophilizer commonly used in the art is also available. Determining the appropriate lyophilizer as well as the effective amount of other lyophilizers is within the scope of routine skill in the art.

The kit may include about 50 μg to about 100 μg, about 100 μg to about 500 μg, about 500 μg to about 1000 μg, about 1 mg to about 10 mg, about 10 mg to about 50 mg, about 50 mg to about 100 mg, about 100 mg to about 500 mg, or about 500 mg to about 1000 mg of the affinity antibody and/or the detection antibody. The kit may include about 50 μg to about 200 μg of the affinity antibody and/or the detection antibody. The kit may include about 100 mg of the affinity antibody and/or the detection antibody. The kit may include about 1 mg of the affinity antibody and/or the detection antibody. The purity of the affinity antibody and/or the detection antibody may be about 90%, 91%, 92%, 93%, 94%, 95%, 96%, 97%, 98%, 99%, or greater than 99%.

The kit may include at least 50 μg to at least 100 μg, at least 100 μg to at least 500 μg, at least 500 μg to at least 1000 μg, at least 1 mg to at least 10 mg, at least 10 mg to at least 50 mg, at least 50 mg to at least 100 mg, at least 100 mg to at least 500 mg, or at least 500 mg to at least 1000 mg of the affinity antibody and/or the detection antibody. The kit may include at least 50 μg to at least 200 μg of the affinity antibody and/or the detection antibody. The kit may include at least 100 mg of the affinity antibody and/or the detection antibody. The kit may include at least 1 mg of the affinity antibody and/or the detection antibody. The purity of the affinity antibody and/or the detection antibody may be at least 90%, 91%, 92%, 93%, 94%, 95%, 96%, 97%, 98%, 99%, or greater than 99%.

The kit may include at least about 50 μg to at least about 100 μg, at least about 100 μg to at least about 500 μg, at least about 500 μg to at least about 1000 μg, at least about 1 mg to at least about 10 mg, at least about 10 mg to at least about 50 mg, at least about 50 mg to at least about 100 mg, at least about 100 mg to at least about 500 mg, or at least about 500 mg to at least about 1000 mg of the affinity antibody and/or the detection antibody. The kit may include at least about 50 μg to at least about 200 μg of the affinity antibody and/or the detection antibody. The kit may include at least about 100 mg of the affinity antibody and/or the detection antibody. The kit may include at least about 1 mg of the affinity antibody and/or the detection antibody. The purity of the affinity antibody and/or the detection antibody may be at least about 90%, 91%, 92%, 93%, 94%, 95%, 96%, 97%, 98%, 99%, or greater than 99%.

In some embodiments, the lyophilized affinity antibody and/or detection antibody may be reconstituted in about 50 μg to about 100 μL, about 100 μg to about 150 μL, about 150 μg to about 200 μL, about 200 μg to about 250 μL, about 250 μg to about 300 μL, about 300 μg to about 350 μL, about 350 μg to about 400 μL, about 400 μg to about 450 μg, about 350 μg to about 400 μL, about 400 μg to about 450 μL, or about 450 μg to about 500 μL of a reconstitution buffer.

In some embodiments, the lyophilized affinity antibody and/or detection antibody may be reconstituted in at least 50 μg to at least 100 μL, at least 100 μg to at least 150 μL, at least 150 μg to at least 200 μL, at least 200 μg to at least 250 μL, at least 250 μg to at least 300 μL, at least 300 μg to at least 350 μL, at least 350 μg to at least 400 μL, at least 400 μg to at least 450 μg, at least 350 μg to at least 400 μL, at least 400 μg to at least 450 μL, or at least 450 μg to at least 500 μL of a reconstitution buffer.

In some embodiments, the lyophilized affinity antibody and/or detection antibody may be reconstituted in at least about 50 μg to at least about 100 μL, at least about 100 μg to at least about 150 μL, at least about 150 μg to at least about 200 μL, at least about 200 μg to at least about 250 μL, at least about 250 μg to at least about 300 μL, at least about 300 μg to at least about 350 μL, at least about 350 μg to at least about 400 μL, at least about 400 μg to at least about 450 μg, at least about 350 μg to at least about 400 μL, at least about 400 μg to at least about 450 μL, or at least about 450 μg to at least about 500 μL of a reconstitution buffer.

The reconstitution buffer may comprise any components known to one of skill in the art. For example, the reconstitution buffer may comprise water, PBS, DMSO, glycerol, or sodium azide. The reconstitution buffer may comprise about 10% to about 15%, about 15% to about 20%, about 20% to about 25%, about 25% to about 30%, or about 30% to about 35% glycerol. The reconstitution buffer may comprise about 0.02% to about 0.04%, about 0.04% to about 0.06%, about 0.06% to about 0.08%, about 0.08% to about 0.1%, about 0.1% to about 0.12%, about 0.12% to about 0.14%, or about 0.14% to about 0.16% sodium azide. In some embodiments, the reconstitution buffer has a pH of about 6 to about 7 or about 7 to about 8. In some embodiments, the reconstitution buffer has a pH of about 7.2.

The reconstitution buffer may comprise at least 10% to at least 15%, at least 15% to at least 20%, at least 20% to at least 25%, at least 25% to at least 30%, or at least 30% to at least 35% glycerol. The reconstitution buffer may comprise at least 0.02% to at least 0.04%, at least 0.04% to at least 0.06%, at least 0.06% to at least 0.08%, at least 0.08% to at least 0.1%, at least 0.1% to at least 0.12%, at least 0.12% to at least 0.14%, or at least 0.14% to at least 0.16% sodium azide. In some embodiments, the reconstitution buffer has a pH of at least 6 to at least 7 or at least 7 to at least 8. In some embodiments, the reconstitution buffer has a pH of at least 7.2.

The reconstitution buffer may comprise at least about 10% to at least about 15%, at least about 15% to at least about 20%, at least about 20% to at least about 25%, at least about 25% to at least about 30%, or at least about 30% to at least about 35% glycerol. The reconstitution buffer may comprise at least about 0.02% to at least about 0.04%, at least about 0.04% to at least about 0.06%, at least about 0.06% to at least about 0.08%, at least about 0.08% to at least about 0.1%, at least about 0.1% to at least about 0.12%, at least about 0.12% to at least about 0.14%, or at least about 0.14% to at least about 0.16% sodium azide. In some embodiments, the reconstitution buffer has a pH of at least about 6 to at least about 7 or at least about 7 to at least about 8. In some embodiments, the reconstitution buffer has a pH of at least about 7.2.

In some embodiments, the kit includes a reconstitution buffer. In some embodiments, the kit includes an affinity antibody and/or a detection antibody that is reconstituted.

The kit may include about 1 μg/mL to about 10 μg/mL, about 10 μg/mL to about 50 μg/mL, about 50 μg/mL to about 100 μg/mL, about 100 μg/mL to about 500 μg/mL, or about 500 μg/mL to about 1000 μg/mL, about 1 mg/mL to about 10 mg/mL, about 10 mg/mL to about 50 mg/mL, about 50 mg/mL to about 100 mg/mL, about 100 mg/mL to about 1000 mg/mL, or about 1000 mg/mL to about 10000 mg/mL of the detection antibody and/or affinity antibody in solution.

The kit may include at least 1 μg/mL to at least 10 μg/mL, at least 10 μg/mL to at least 50 μg/mL, at least 50 μg/mL to at least 100 μg/mL, at least 100 μg/mL to at least 500 μg/mL, or at least 500 μg/mL to at least 1000 μg/mL, at least 1 mg/mL to at least 10 mg/mL, at least 10 mg/mL to at least 50 mg/mL, at least 50 mg/mL to at least 100 mg/mL, at least 100 mg/mL to at least 1000 mg/mL, or at least 1000 mg/mL to at least 10000 mg/mL of the detection antibody and/or affinity antibody in solution.

The kit may include at least about 1 μg/mL to at least about 10 μg/mL, at least about 10 μg/mL to at least about 50 μg/mL, at least about 50 μg/mL to at least about 100 μg/mL, at least about 100 μg/mL to at least about 500 μg/mL, or at least about 500 μg/mL to at least about 1000 μg/mL, at least about 1 mg/mL to at least about 10 mg/mL, at least about 10 mg/mL to at least about 50 mg/mL, at least about 50 mg/mL to at least about 100 mg/mL, at least about 100 mg/mL to at least about 1000 mg/mL, or at least about 1000 mg/mL to at least about 10000 mg/mL of the detection antibody and/or affinity antibody in solution.

In some embodiments, the second affinity label is streptavidin. In some embodiments, the kit includes a solid phase, a second affinity label, and instructions for coating the solid phase with the second affinity label.

The solid phase may be any material known to one of skill that is imageable and capable of binding a second affinity label, including streptavidin. For example, the substrate may be a slide, plate, dish, or flask. In some embodiments, the kit comprises a solid phase that is coated with streptavidin. In some embodiments, the kit comprises a solid phase, streptavidin, and instructions for coating the solid phase with streptavidin.

The kit may comprise one or more buffers. In some embodiments, the kit comprises an acid wash buffer. In some embodiments, the kit further comprises a washing buffer. In some embodiments, the kit comprises a blocking buffer. The buffers of the present technology may be formulated according to any of the formulations of this technology or the formulations known to one of skill in the art.

In accordance with some embodiments, the kit may contain one or more consumables for performing the methods of the present technology. For example, the kit may include one or more plates containing a plurality of wells. In some embodiments, the plate may include 6, 9, 12, 16, 24, 36, 96, 384, or 1536 wells. In some embodiments, a kit or system includes 1, 2, 3, 4, 5, 6, 7, 8, 9, 10, 11, 12, 13, 14, 15, 16, 17, 18, 19, 20, or more than twenty assay plates.

The kit may also include instructions for performing all or part of the methods described in the present technology. In some embodiments, the kit includes links (e.g., URLs or QR codes) that direct a user to instructions for performing all or part of the methods of the present technology. The kit may include software or other computer technology to facilitate performing the methods of the present technology.

The components of the kit may be stored to provide stability to the particular components. For example, components of the kit may be stored in a light-impermeable container if the component comprises a light-sensitive compound. In some embodiments, the components of the kit may be stored in an airtight container. In some embodiments, the components of the kit are stored frozen, in liquid form, or in solid form. In some embodiments, the components of the kit are stored at about −195° C., −80° C., −20° C., 4° C., or 20° C.

In some embodiments, the kit comprises: (i) an affinity antibody comprising biotin; (ii) a detection antibody comprising a sulfo-Tag; (iii) a plate comprising streptavidin; (iv) TPA; and (v) a wash buffer.

As used herein, the word “about” when immediately preceding a numerical value means a range of plus or minus 10% of that value, e.g., “about 50” means 45 to 55, “about 10” means “9 to 11”, etc. Furthermore, the phrases “less than about” a value or “greater than about” a value should be understood in view of the definition of the term “about” provided herein.

In another embodiment, the kit includes one or more of the components, consumables, or reagents described in the examples below.

### EXAMPLES

Examples provided in this technology include example methods for detecting ADAs in human samples with an ECL assay, methods of validating the assay, and results from the validation.

List of abbreviations and definitions: ADA: Anti-Drug Antibody; A.T.: Ambient Temperature; Bio-XTMAB-16: Biotin Labeled XTMAB-16; CCP: Confirmatory Cut-Point; CP: Cut-Point; CV: Coefficient of Variation; diH₂O: Deionized Water; ECL: Electrochemiluminescence; ECLIA: Electrochemiluminescence Immunoassay; HPC: High Positive Control; iNHS: Individual Naïve Human Scrum; LPC: Low Positive Control; MRD: Minimum Required Dilution; MSD: Meso Scale Discovery; NA: Not Applicable; NC: Negative Control; pNHS: Pooled Naïve Human Serum; PBS: Phosphate-Buffered Saline; QC: Quality Control; rECL: Relative ECL; Ru-XTMAB-16: Ruthenium Labeled XTMAB-16; SCP: Screening Cut-Point; S-CPF: Screening Cut-Point Factor; SD: Standard Deviation; TCP: Titration Cut-Point; T-CPF: Titration Cut-Point Factor.

### Example 1: Methods for Detecting ADAs in a Subject by an ECL Assay and Methods of Validating the Same

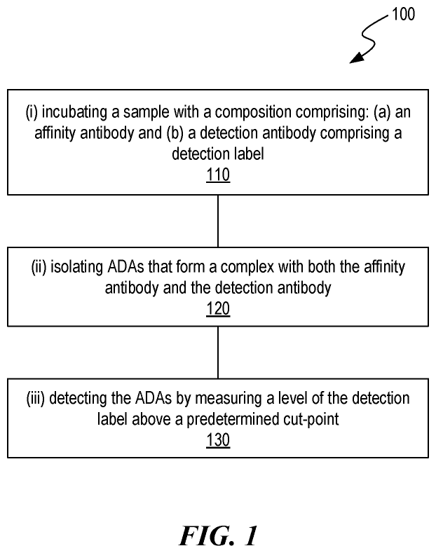

Overview: FIG. 1 illustrates an exemplary method (method 100) for detecting an ADA that binds the target antibody in a sample. Method 100 may be used for immunogenicity assessments of various antibodies, including, but not limited to, anti-TNFα antibodies. Method 100 comprises: (i) incubating the sample with a composition comprising: (a) an anti-TNFα antibody conjugated to a first affinity label (affinity antibody), and (b) an anti-TNFα antibody conjugated to a detection label (detection antibody) 110; (ii) isolating ADAs that form a complex with both the affinity antibody and the detection antibody, wherein the ADA is isolated by contacting the complex with a second affinity label that binds to the first affinity label 120; and (iii) detecting the ADA by measuring a level of the detection label above a predetermined cut-point 130. Isolating the ADAs may comprise: (a) providing a solid phase comprising the second affinity label; (b) contacting the sample and the composition with the solid phase; and (c) washing the solid phase.

Validation Summary: To support an immunogenicity assessment of XTMAB-16 in human samples, methods of detecting antibodies in human serum samples were assessed.

An MSD streptavidin plate was blocked to prevent non-specific antibody binding. The control and experimental samples were incubated with Bio-XTMAB-16 and transferred to the blocked MSD streptavidin plate. Following incubation, the plate was washed and then incubated with Ru-XTMAB-16. After Ru-XTMAB-16 incubation, the MSD streptavidin plate was washed again and read with plate reader (MSD Sector Imager S600). Screening, confirmatory, and titration steps were also incorporated into the validation methods.

Screening methods were used to detect potentially positive ADAs to XTMAB-16 in human serum samples. Confirmatory methods were used to determine whether the potential ADA-positive samples were specific to XTMAB-16. Titration methods were used to provide an estimate of ADA levels in human scrum samples that were confirmed to be positive for antibodies to XTMAB-16.

Materials: The validation methods used the reagents and controls listed in Table 2

TABLE 2
Reagents and controls in ECL validation

| Reagent | Storage Condition |
|---|---|
| Anti-XTMAB-16 antibody | −70° C. ± 10° C. |
| (91F9D4) (0.781 mg/mL) | |
| XTMAB-16 Drug | −70° C. ± 10° C. |
| (100 mg/vial) | |
| Bio-XTMAB-16 | −70° C. ± 10° C. |
| (1.234 mg/mL) | |
| Ru-XTMAB-16 | −70° C. ± 10° C. |
| (1.212 mg/mL) | |
| MSD Streptavidin Plate | 2° C.-8° C. |
| pNHS | −70° C. ± 10° C. |
| HPC 4000.0 ng/mL | −70° C. ± 10° C. |
| LPC 9.70 ng/mL | −70° C. ± 10° C. |
| iNHS | −70° C. ± 10° C. |
| NC 0 ng/mL | −70° C. ± 10° C. |
| PBS-Tween tablet | A.T. |
| 0.1M Glycine Buffer pH 3.0 | A.T. |
| Next Generation Buffer I | 2° C.-8° C. |
| (NGBI) | |
| PBS | 2° C.-8° C. |
| MSD Read Buffer 4X | A.T. |
| MSD Streptavidin Plate | 2° C.-8° C. |

Negative Controls: A pooled lot of pNHS was prepared by pooling together equal volumes of 40 lots of iNHS (29 male lots and 11 female lots).

High and Low Positive Controls: An ECL assay was validated for detection of anti-XTMAB-16 antibodies in human serum and to determine whether potentially ADA-positive samples were specific to anti-XTMAB-16. A titration method was incorporated to provide a quasi-quantitative estimate of ADA level in human serum samples that were confirmed as positive for having anti-XTMAB-16. Preparation of positive controls is further detailed in Table 3.

TABLE 3
Preparation of Positive Controls

| | | | | | | Resulting |
|---|---|---|---|---|---|---|
| | | Stock | NC | Total | | Concentration |
| ADA | ADA | Volume | Volume | Volume | Resulting | Volume |
| Tube ID | Concentration | (μL) | (μL) | (μL) | Tube | (ng/mL) |
| PC Stock | 781,000 | 5.0 | 73.1 | 78.1 | Int-1 | 50,000 |
| Int-1 | 50,000 | 48 | 552 | 600 | HPC | 4,000 |
| HPC | 4,000 | 5.1 | 2095 | 2100 | LPC | 9.70 |

Reagent Preparation: The validation PCs were prepared by adding the anti-XTMAB-16 antibody to the pNHS at high (4,000.0 ng/ml) and low (9.70 ng/mL) concentrations for HPC and LPC, respectively.

Reagents were prepared, including MSD 2× Read Buffer (4×MSD Read Buffer diluted with diH₂O), Wash Buffer (1×PBS with 0.05% Tween-20), Blocking Buffer (Protein-Free), Assay Buffer (NGBI), and XTMAB-16 Drug Stock Solution (XTMAB-16 dissolved in PBS to 10 mg/mL).

QC preparation included preparation of an NC (pNHS made from at least 40 individual lots) and LPC and HPC positive controls. HPC concentration was 4,000 ng/ml and the LPC final concentration was prepared at a 99.9% confidence interval.

Establishment of LPC Concentration: The LPC concentration was calculated in both screening and confirmatory assay using the mean of the corresponding sensitivity+t_{0.99, df}×SD (sensitivity). The final LPC concentration was the higher LPC concentration between the screening LPC and the confirmatory LPC. Each sensitivity will be obtained from a separate sensitivity run. The screening sensitivity concentration will be interpolated from the screening sensitivity curve at the SCP. The confirmatory sensitivity concentration will be interpolated from the confirmatory sensitivity curve at the CCP. Once the LPC has been finalized, at least two sets of LPCs and HPCs and at least four sets of NCs will be included as plate controls in each run.

System Suitability Criteria: System suitability will be assessed from all accepted validation runs (except the robustness run and the long-term stability run, if any) to set up assay acceptance ranges for the NC, LPC, and HPC. The control ranges will be set using the response values of NC, LPC, and HPC, as detailed below.

Examples of control range calculations:

$$Upper limit of NC = Mean of NC responses + 3 \times SD of NC responses$$

$$LPC range = Mean of LPC responses \pm 3 \times SD of LPC responses$$

$$Lower limit of HPC = Mean of HPC responses - 3 \times SD of HPC responses$$

In cut-point and sensitivity runs, at least 50% of each level of HPC must have % CV of the replicates ≤20.0%.

After LPC concentration is finalized, at least 50% of each level of plate controls must have: % CV of ≤20.0%.

The relationship of the accepted control samples should be as follows:
For screening and titration assays, the mean ECL: NC<SCP (if applicable)≤LPC (if applicable)<HPC
For confirmatory assay, the % inhibition: NC<CCP (if applicable)≤LPC (if applicable) and HPC.

To accept a run, at least 50% of each level of control samples and at least 66.7% of total control samples should meet the above criteria.

Screening Assay Plate Control Acceptance Criteria: For the screening assay, the CV of each plate control was 20.0% or less, and plate controls were present in duplicates. The plate NC was considered the mean of all accepted NCs, and % CV failure and outliers were excluded. The relationship of accepted control samples in the screening assays were mean response (ECL): NC<SCP≤LPC<HPC, and the ECL was within the range established during validation:

NC: ECL mean≤158.8; LPC: 95.5≤ECL mean≤156.1; HPC: 4761.7≤ECL mean.

The S-CPF was 1.10, and the plate-specific SCP was the value calculated from the NC multiplied by the S-CPF.

Confirmatory Assay Plate Control Acceptance Criteria: For the confirmatory assay, the CV of each plate control was 20.0% or less, and plate controls were present in duplicates. The relationship of the accepted control samples were % inhibition: NC<CCP≤LPC and HPC. Plate NC was the mean of all accepted NCs, and % CV failure and outliers were excluded. The CCP was 16.45% in the presence of XTMAB-16, and % inhibition was calculated by the following formula:

$$\% inhibition = 100 * ( 1 - [ Mean ECL of spiked samples / Mean ECL of unspiked samples ] )$$

Titration Assay Plate Control Acceptance Criteria: For the titration assay, the CV of each plate control was 20.0% or less, and plate controls were present in duplicates. The plate NC was considered the mean of all accepted NCs, and % CV failure and outliers were excluded. The relationship of accepted control samples in the titration assays were mean response (ECL): NC<SCP≤LPC<HPC, and the ECL was within the range established during validation:

NC: ECL mean≤158.8; LPC: 95.5≤ECL mean≤156.1; HPC: 4761.7≤ECL mean.

Titer control was 25600≤titer for XTMAB-16 ADA PC at 4,000 mg/mL≤102,400.

The T-CPF was 1.20, and the plate-specific TCP was calculated by multiplying the T-CPF by the plate NC.

Screening Assay Sample Acceptance Criteria: The mean ECL values for screening samples had a % CV of 20.0% or less. Samples with mean ECL values≥plate-specific SCP were classified as potentially positive. Samples with mean ECL values less than the plate-specific SCP were classified as negative.

Confirmatory Assay Sample Acceptance Criteria: The mean ECL values for confirmatory samples had a % CV of 20.0% or less. Samples were classified as positive for anti-XTMAB-16 antibodies if the following conditions were met: (1) the mean ECL for the sample in the screening assay was ≥plate-specific SCP and (2) the % inhibition in the confirmatory assay was ≥CCP. Samples were classified as negative for anti-XTMAB-16 antibodies if any of the following conditions were met: (1) the mean ECL in the screening assay<plate-specific SCP and (2) the % inhibition in the confirmatory assay<CCP.

Titration Assay Sample Acceptance Criteria: The titer of undiluted samples with an ECL below plate TCP were reported as MRD. The titer of undiluted samples with an ECL above the TCP, but where the first dilution (minimum dilution) had an ECL below the plate TCP, were reported as MRD. For samples with at least one dilution that reaches an ECL below the TCP, the diluted sample must have at least two dilutions spanning the TCP with a % CV≤20.0%.

The titer of a diluted sample with ECL above the plate-specific TCP was considered the dilution factor of the last dilution sample with ECL above the plate-specific TCP.

Determination of Cut-Point: SCP: The ECL signal of each individual sample was normalized by the plate NC (expressed as S/N). The data was then log-transformed. After outlier removal, normality test was performed on the data using JMP. The S-CPF was calculated based on 5% false-positive rate using the equation below based on the data distribution.

$$S - CPF = “ Mean + 1.645 \times SD ” ( if data normally distributed ) or “ {95}^{th} percentile ” ( if data not normally distributed )$$

A plate-specific SCP will be calculated using the following formula:

$$SCP = Mean NC ECL values in a plate * S - CPF$$

CCP: CCP was defined by the inhibition rate from individual samples with and without spiking reference drug. The % inhibition and CCP, using a 1% false-positive rate, are calculated using the equation below based on the data distribution by JMP.

$$\% inhibition = 100 * ( {ECL}_{without drug} - {ECL}_{with drug} ) / {ECL}_{without drug}$$

$$CCP = “ Mean \% inhibition + 2.33 * SD \% inhibition ” ( if data normally distributed ) or “ {99}^{th} Percentile ” ( if data not normally distributed )$$

TCP: T-CPF will be calculated using the S-CPF normalized data. T-CPF will be calculated based on 0.1% false-positive rate using the equation below based on the data distribution. T-CPF will be reported to two decimal places.

$$T - CPF = “ Mean + 3.09 \times SD ” ( if data normally distributed ) or “ {99.9}^{th} percentile ” ( if data not normally distributed )$$

$$Plate TCP = Mean NC ECL values in a plate * T - CPF$$

Experimental design and target specifications for determining cut-point are outlined in Table 4.

TABLE 4
Experimental Design and Target Specifications for Determining Cut-Point

| | Experimental Design | Target Specifications |
|---|---|---|
| S-CPF/T-CPF | At least 50 individual | Individual samples demonstrating |
| | serum samples | a % CV >20.0% will be excluded from the |
| | n: 6 minimum | final data set and will not be included in the |
| | Replicates: 2 | assay cut-point calculations |
| | Analysts: 2 minimum | At least 66.7% of the data for |
| | Days: 2 minimum | individual lots of sera should be included in |
| | | the assay cut-point factor calculations. Failed |
| | | lots due to % CV >20.0% will be reanalyzed |
| | | until at least 66.7% of the data for individual |
| | | lots of sera have valid results |
| CCP | At least 50 individual | Individual samples demonstrating |
| | serum samples incubated | a % CV >20.0% will be excluded from the |
| | with the drug (XTMAB-16). | final data set and will not be included in the |
| | n: 6 minimum | assay cut-point calculations |
| | Replicates: 2 | Calculate the % inhibition using the |
| | Analysts: 2 minimum | following formula: |
| | Days: 2 minimum | % Signal Inhibition = 100*[1 − (Response of |
| | | sample competed with drug/Response of |
| | | sample)] |
| | | At least 66.7% of the data for |
| | | individual lots of sera should be included in |
| | | the assay cut-point factor calculations. Failed |
| | | lots due to % CV >20.0% will be reanalyzed |
| | | until at least 66.7% of the data for individual |
| | | lots of sera have valid results |

Sensitivity: Sensitivity of ADA assays may be defined by the lowest concentration at which a positive control antibody preparation consistently provides a positive signal in the assay. Assay sensitivity was determined in the presence and absence of drug. The experimental design for the sensitivity assay for the screening assay is outlined in Table 5, and for the confirmatory assay in Table 6.

TABLE 5
Screening Assay Experimental Design and Target
Specifications for Sensitivity Assay

| | Experimental Design | Target Specifications |
|---|---|---|
| Assay | ADA PCs (4000 ng/mL) prepared | Plate acceptance criteria is |
| sensitivity in | by spiking ADA PC into pooled | met |
| the | matrix and then two-fold serially | At least 66.7% of sensitivity |
| screening | diluted. At least 6 dilutions are | runs will be used in |
| assay | suggested. At least one dilution | estimating the assay |
| | with ECL below the plate SCP will | screening sensitivity |
| | be included in the curve fitting for | Assay screening sensitivity |
| | assay sensitivity evaluation. | will be the mean |
| | Replicates: 2 | |
| | n: 1 minimum for 3 individuals, 3 | |
| | minimum for 1 pooled matrix | |
| | Analysts: 2 minimum | |
| | Runs: 2 minimum | |

TABLE 6
Confirmatory Assay Experimental Design and
Target Specifications for Sensitivity Assay

| | Experimental Design | Target Specifications |
|---|---|---|
| Assay | ADA PCs (4000 ng/mL) prepared | Plate acceptance criteria is |
| sensitivity in | by spiking ADA PC into pooled | met |
| the | matrix and then two-fold serially | At least 66.7% of sensitivity |
| confirmatory | diluted. At least 6 dilutions are | runs will be used in |
| assay | suggested. At least one dilution | estimating the assay |
| | with % inhibition below the CCP | confirmatory sensitivity |
| | will be included in the curve fitting | Assay confirmatory |
| | for confirmatory sensitivity | sensitivity will be the mean |
| | evaluation. | |
| | Replicates: 2 | |
| | n: 1 minimum for 3 individuals, 3 | |
| | minimum for 1 pooled matrix | |
| | Analysts: 2 minimum | |
| | Runs: 2 minimum | |

Hook Effect: Positive control antibody samples used for prozone assessment was prepared at a concentration level higher than HPC to demonstrate that higher concentrations may not cause a decrease in response. The prepared samples were frozen for a minimum of 12 hours before serial dilution and analysis. On the day of the assay, the sample was diluted using negative serum as diluent before analysis to give concentrations above and at the assay sensitivity level. At least 80% of the validation sample used in hook effect evaluation had a % CV≥20.0%.

Intra-Assay and Inter-Assay Precision: Intra-assay precision was evaluated based on the % CV of the NC, LPC, and HPC using a minimum of six independent preparations of the same sample in one validation run, using both the screening and confirmatory assays. The experimental design and target specifications for the intra-assay precision are outlined in Table 7.

TABLE 7
Intra-Assay Precision in Screening and Confirmatory Assays

| | Experimental Design | Target Specifications |
|---|---|---|
| Intra-assay | NC, LPC, HPC | CV ≤20% between |
| precision in | Replicates: 2 | replicates |
| screening | n: ≥6 sets each for | Mean ECL: NC < SCP ≤ |
| assay | NC, LPC, and HPC | LPC < HPC |
| | Analysts: 1 minimum | At least 6 sets of each plate |
| | Days: 1 minimum | controls will be included in |
| | | the intra-assay precision |
| | | % CV calculation |
| | | Intra-assay CV ≤20% for |
| | | HPC and LPC |
| | | No intra CV criteria for NC |
| Intra-assay | NC, LPC, HPC | CV ≤20% between |
| precision in | Replicates: 2 | replicates |
| confirmatory | n: ≥6 sets each for | Mean ECL: NC < CCP ≤ |
| assay | NC, LPC, and HPC | LPC < HPC |
| | Analysts: 1 minimum | At least 6 sets of each plate |
| | Days: 1 minimum | controls will be included in |
| | | the intra-assay precision |
| | | % CV calculation |
| | | Intra-assay CV ≤20% for |
| | | HPC and LPC |
| | | No intra CV criteria for NC |

Inter-assay precision was evaluated based on the % CV of the NC, LPC, and HPC results across all applicability accepted validation runs, except for the test of robustness and long-term stability. The experimental design and target specifications for the inter-assay precision are outlined in Table 8.

TABLE 8
Inter-Assay Precision in Screening and Confirmatory Assays

| | Experimental Design | Target Specifications |
|---|---|---|
| Inter-assay | All NC, LPC, HPC accepted in | CV ≤20% between |
| precision in | validation runs, except robustness | replicates |
| screening | runs | Mean ECL: NC < SCP ≤ |
| assay | Replicates: 2 | LPC < HPC |
| | Runs: 6 minimum | CV ≤20% for HPC and LPC |
| | Analysts: 2 minimum | No inter CV criteria for NC |
| | Days: 2 minimum | |
| Intra-assay | All NC, LPC, HPC accepted in | CV ≤20% between |
| precision in | validation runs, except robustness | replicates |
| confirmatory | runs | Mean ECL: NC < SCP ≤ |
| assay | Replicates: 2 | LPC < HPC |
| | Runs: 6 minimum | CV ≤20% for HPC and LPC |
| | Analysts: 2 minimum | No inter CV criteria for NC |
| | Days: 2 minimum | |

Selectivity and Matrix Interference: The selectivity and matrix interference for individual normal matrix was evaluated by spiking ADA at LPC level to at least 10 individual normal matrixes in both screening and confirmatory tests. The selectivity and matrix interference for individual diseased matrix was evaluated by spiking ADA at LPC level to at least 10 individual diseased matrixes in both screening and confirmatory tests. In the screening assay, ADA spiked and non-ADA spiked matrix samples were analyzed in comparison to SCP. In the confirmatory assay, the analyzed samples will be compared with CCP. Samples may be frozen prior to analysis. The selectivity samples confirmed with pre-existing antibodies were excluded in evaluating assay selectivity. The selectivity evaluation experimental design and target specifications are outlined in Table 9.

TABLE 9
Selectivity Evaluation Experimental Design and Target Specifications

| | Experimental Design | Target Specifications |
|---|---|---|
| Selectivity | Minimum of 10 individual samples | Plate acceptance criteria is |
| | of normal matrix unspiked (blank) | met |
| | and spiked with ADA PC at LPC | The method selectivity is |
| | level. An LPC spiked normal | considered acceptable if no |
| | pooled serum may be used as a | more than 20% of the |
| | reference. | unspiked samples report as |
| | N: minimum 10 samples (spiked | positive, and at least 80% of |
| | and unspiked) | the spiked samples report as |
| | Replicates: 2 | positive |
| | Runs: 1 minimum | Samples are reported as |
| | Minimum of 10 individual samples | positive only if the below |
| | of diseased matrix unspiked | criteria are met: |
| | (blank) and spiked with ADA PC | (1) screening assay ECL ≥ |
| | at LPC level. An LPC spiked | SCP, (2) confirmatory assay |
| | normal pooled serum may be used | % inhibition ≥ CCP, (3) |
| | as a reference. | precision (expressed as |
| | | % CV) has CV value ≤20.0% |
| | | between replicates |

Haemolyzed and Lipemic Matrix Interference: The selectivity assessment includes haemolyzed (2%) and lipemic (150 mg/dL) matrix assessment. A minimum of three control matrix pooled serum samples for each condition were assessed unspiked and spiked at LPC level. The spiked matrix samples will be analyzed in comparison to plate SCP. In confirmatory assays, the analyzed samples were compared with CCP. Samples may be frozen prior to analysis. The matrix interference experimental design and target specifications are detailed in Table 10.

TABLE 10
Matrix Interference Experimental Design and Target Specifications

| | Experimental Design | Target Specifications |
|---|---|---|
| Matrix | Minimum of 3 pooled lot of 2% | Plate acceptance criteria is |
| interference | haemolyzed sample, minimum of 3 | met |
| | pooled lot of 150 mg/dL lipemic | Method selectivity is |
| | sample, unspiked (blank) and | considered acceptable if |
| | spiked with ADA PC at LPC level | (1) no more than 20% of |
| | n: minimum 3 for haemolyzed and | the unspiked samples |
| | lipemic, respectively | report as negative, and (2) |
| | Replicates: 2 | at least 80% of the spiked |
| | Runs: 1 minimum | samples report as positives |
| | | Samples are reported as |
| | | positive if the below |
| | | criteria are met: |
| | | (1) screening assay ECL ≥ |
| | | SCP, (2) confirmatory |
| | | assay % inhibition ≥ CCP, |
| | | (3) precision (expressed as |
| | | % CV) ≤20.0% between |
| | | replicates |

Determination of Drug Tolerance: Drug tolerance is to determine the highest level of drug that may be added to a sample spiked with the positive control antibodies without interfering with its detection as positive antibody response. Various drug concentrations were spiked into matrix samples containing various concentrations of positive control antibodies.

Drug tolerance limit may be defined as the highest level of drug in the presence of which ADA is still detected. ADA PC at 100 ng/ml and LPC level will be tested. The highest drug concentration that retains a positive response in the screening assay for each ADA level tested will be defined as the drug tolerance level of the screening assay. The experimental design and target specifications for testing drug tolerance are outlined in Table 11.

TABLE 11
Experimental Design and Target Specifications for Testing Drug Tolerance

| | Experimental Design | Target Specifications |
|---|---|---|
| Selectivity | Positive control samples at various | Plate acceptance criteria is met |
| | ADA concentrations mixed with | At least 80% of samples above the |
| | drug samples at various | SCP have CV ≤20% between |
| | concentration levels, including a | replicates |
| | 0 ng/mL drug level. | The samples with highest drug |
| | ADA PC at 100 ng/mL and LPC | concentration that still gives ECL |
| | level with different concentration | result above SCP will be reported |
| | levels of drug (400, 200, 100, 50, | as drug tolerance |
| | 20, 10, 5, and 0 μg/mL). | |
| | n: 1 minimum | |
| | Replicates: 2 | |
| | Runs: 1 minimum | |

Stability Test: Combined stressed samples with bench-top (24±6 hours or 8±2 hours) and freeze/thaw (6 or 3 cycles from −70° C.±10° C. to ambient temperature) were tested. The samples were prepared and stored at −70° C.±10° C. for overnight (at least 12 hours) before being thawed at ambient temperature for the first cycle. For the subsequent freeze/thaw cycles, samples were frozen for at least 12 hours under the same conditions and then thawed. Samples were left at the bench-top for 2±1 hours before refreezing. The experimental design and target specifications are outlined in Table 12.

TABLE 12
Experimental Design and Target Specifications
for Bench-Top and Freeze/Thaw Stability Assays

| Parameter | Experimental Design | Target Specification |
|---|---|---|
| Bench-top, | NC, LPC, and HPC | System acceptance criteria is met |
| freeze/thaw | n: ≥3 | Plate acceptance criteria is met |
| stability | | % CV ≤20.0% between replicates |
| | | Confirmatory assay: |
| | | % inhibition of NC < CCP ≤ LPC and HPC |
| | | Screening assay: |
| | | Mean ECL of NC < SCP ≤ LPC < HPC |
| | | At least 66.7% stability samples at each |
| | | level must meet above acceptance criteria. |

Data Evaluation: ECL count raw data was captured using the MSD Sector Imager S600 and Discovery Workbench Software Version 4.0. % CV, SD, and % inhibition were each calculated. Statistical evaluation of cut-point factors was conducted using JMP software.

Assay Performance Characteristics: All samples and controls, including two sets of LPC and HPC and at least four sets of NC, were evaluated in duplicate. The text file imported from the MSD Sector Imager S600 was used as the source data for analysis and verified against the screen capture of the original data. The source data was processed in Microsoft Excel to calculate mean of ECL values, % CV, and % inhibition. The S-CPF, T-CPF, and CCPs were calculated using JMP 14 statistical software. All mean duplicate or quadruplicate ECL values were assessed in the analysis.

Failed tests: Plate control testing resulting in failed runs was repeated twice. If the repeated run failed again and failure was not due to the experimental error, the method was investigated and the method was revised or reoptimized with runs repeated, where necessary.

### Example 2: Validation of Method of Detecting ADAs in Serum Samples Using an Electrochemiluminescence Immunoassay

Cut-Point Determination: To determine the screening, confirmatory, and titration assay cut-point factors for the detection of anti-XTMAB-16 antibodies, two serum samples from 54 lots of naïve male and female human serum were tested over five days. Each sample was tested six times across 18 individual runs.

Cut-point statistical outliers were determined using quantile range outlier's method, and identified outliers were removed. Cut-point normality values were tested by Shapiro-Wilk W Test using JMP software.

For each run, the mean ECL signal of each individual serum sample was normalized using the mean ECL of a plate NC. The normalized ECL (rECL) was log-transformed. Examples of data analyzed according to the present technology are shown in Tables 12-14, for example. Table 12 shows raw data from one run of samples that were tested according to the methods of the present technology. Table 13 shows raw data for S-CPF and T-CPF for the runs in Table 12. Table 14 illustrates an example of the summarized normalized data for S-CPF and T-CPF calculation.

TABLE 12
Example raw data from a single run

| Example | | |
|---|---|---|
| Run | Validation Parameter | Result |
| 34 | Screening/Confirmation Cut Point (S01-S18) | Pass |
| 35 | Screening/Confirmation Cut Point (S19-S36) | Pass |
| 36 | Screening/Confirmation Cut Point (S37-S54) | Pass |
| 37 | Screening/Confirmation Cut Point (S01-S18) | Pass |
| 38 | Screening/Confirmation Cut Point (S19-S36) | Pass |
| 39 | Screening/Confirmation Cut Point (S37-S54) | Pass |
| 40 | Screening/Confirmation Cut Point (S01-S18) | Pass |
| 41 | Screening/Confirmation Cut Point (S19-S36) | Pass |
| 42 | Screening/Confirmation Cut Point (S37-S54) | Pass |
| 43 | Screening/Confirmation Cut Point (S01-S18) | Pass |
| 44 | Screening/Confirmation Cut Point (S19-S36) | Pass |
| 45 | Screening/Confirmation Cut Point (S37-S54) | Pass |
| 46 | Screening/Confirmation Cut Point (S01-S18) | Void |
| 47 | Screening/Confirmation Cut Point (S19-S36) | Void |
| 48 | Screening/Confirmation Cut Point (S37-S54) | Void |
| 49 | Screening/Confirmation Cut Point (S01-S18) | Pass |
| 50 | Screening/Confirmation Cut Point (S19-S36) | Pass |
| 51 | Screening/Confirmation Cut Point (S37-S54) | Pass |
| 52 | Screening/Confirmation Cut Point (S01-S18) | Pass |
| 53 | Screening/Confirmation Cut Point (S19-S36) | Pass |
| 54 | Screening/Confirmation Cut Point (S37-S54) | Pass |
| 55 | Individual Sensitivity-01 | Pass |
| 56 | Individual Sensitivity-02 | Pass |
| 57 | Individual Sensitivity-03 | Pass |
| 58 | Pooled Sensitivity-01 | Pass |
| 59 | Pooled Sensitivity-02 | Pass |
| 60 | Pooled Sensitivity-03 | Pass |
| 61 | Robustness-Maximum | Pass |
| 62 | Robustness-Minimum | Pass |
| 63 | Intra-Run Precision | Pass |
| 64 | Hook Effect | Pass |
| 65 | Drug Tolerance | Pass |
| 66 | Bench-Top Stability | Pass |
| 67 | Freeze-Thaw Stability | Pass |
| 68 | Selectivity-1 | Pass |
| 69 | Selectivity-2 | Pass |
| 70 | Selectivity-3 | Pass |
| 71 | Selectivity-4 | Pass |

TABLE 13
S-CPF and T-CPF raw data

| Example | | | | | | |
|---|---|---|---|---|---|---|
| Run* | Rep 1 | Rep 2 | Mean | % CV | Plate NC | rECL |
| 34 | 94 | 88 | 91.0 | 4.7 | 89.4 | 1.0179 |
| | 86 | 83 | 84.5 | 2.5 | | 0.9452 |
| | 93 | 86 | 89.5 | 5.5 | | 1.0011 |
| | 91 | 86 | 88.5 | 4.0 | | 0.9899 |
| | 82 | 80 | 81.0 | 1.7 | | 0.9060 |
| | 95 | 91 | 93.0 | 3.0 | | 1.0403 |
| | 77 | 73 | 75.0 | 3.8 | | 0.8389 |
| | 79 | 76 | 77.5 | 2.7 | | 0.8669 |
| | 79 | 76 | 77.5 | 2.7 | | 0.8669 |
| | 79 | 82 | 80.5 | 2.6 | | 0.9004 |
| | 78 | 85 | 81.5 | 6.1 | | 0.9116 |
| | 92 | 90 | 91.0 | 1.6 | | 1.0179 |
| | 89 | 81 | 85.0 | 6.7 | | 0.9508 |
| | 88 | 82 | 85.0 | 5.0 | | 0.9508 |
| | 81 | 81 | 81.0 | 0.0 | | 0.9060 |
| | 89 | 84 | 86.5 | 4.1 | | 0.9676 |
| | 83 | 80 | 81.5 | 2.6 | | 0.9116 |
| | 87 | 84 | 85.5 | 2.5 | | 0.9564 |
| 35 | 81 | 83 | 82.0 | 1.7 | 80.3 | 1.0212 |
| | 76 | 77 | 76.5 | 0.9 | | 0.9527 |
| | 84 | 88 | 86.0 | 3.3 | | 1.0710 |
| | 85 | 87 | 86.0 | 1.6 | | 1.0710 |
| | 83 | 89 | 86.0 | 4.9 | | 1.0710 |
| | 83 | 88 | 85.5 | 4.1 | | 1.0648 |
| | 80 | 77 | 78.5 | 2.7 | | 0.9776 |
| | 80 | 77 | 78.5 | 2.7 | | 0.9776 |
| | 78 | 73 | 75.5 | 4.7 | | 0.9402 |
| | 81 | 82 | 81.5 | 0.9 | | 1.0149 |
| | 80 | 87 | 83.5 | 5.9 | | 1.0399 |
| | 79 | 82 | 80.5 | 2.6 | | 1.0025 |
| | 78 | 82 | 80.0 | 3.5 | | 0.9963 |
| | 85 | 88 | 86.5 | 2.5 | | 1.0772 |
| | 76 | 74 | 75.0 | 1.9 | | 0.9340 |
| | 79 | 75 | 77.0 | 3.7 | | 0.9589 |
| | 75 | 75 | 75.0 | 0.0 | | 0.9340 |
| | 81 | 81 | 81.0 | 0.0 | | 1.0087 |
| 36 | 78 | 79 | 78.5 | 0.9 | 77.8 | 1.0090 |
| | 67 | 76 | 71.5 | 8.9 | | 0.9190 |
| | 88 | 84 | 86.0 | 3.3 | | 1.1054 |
| | 86 | 81 | 83.5 | 4.2 | | 1.0733 |
| | 83 | 82 | 82.5 | 0.9 | | 1.0604 |
| | 83 | 79 | 81.0 | 3.5 | | 1.0411 |
| | 76 | 71 | 73.5 | 4.8 | | 0.9447 |
| | 74 | 71 | 72.5 | 2.9 | | 0.9319 |
| | 73 | 69 | 71.0 | 4.0 | | 0.9126 |

*Each row represents a different lot

TABLE 14
S-CPF and T-CPF raw data

| Screening 1* | Screening 2 | Screening 3 | Screening 4 | Screening 5 | Screening 6 |
|---|---|---|---|---|---|
| 1.0179 | 0.9988 | 0.9880 | 1.0205 | 0.9793 | 0.9721 |
| 0.9452 | 1.0229 | 0.9398 | 1.3406 | 1.1463 | 1.0510 |
| 1.0011 | 1.0349 | 1.1325 | 1.0266 | 1.0023 | 1.1543 |
| 0.9899 | 1.0168 | 1.0843 | 1.0145 | 0.9735 | 1.0875 |
| 0.9060 | 0.9687 | 1.0361 | 0.9541 | 0.9620 | 1.0510 |
| 1.0403 | 1.0048 | 1.0241 | 1.0326 | 1.0023 | 1.0693 |
| 0.8389 | 0.9146 | 0.9880 | 0.9722 | 1.0023 | 0.9235 |
| 0.8669 | 0.9386 | 0.9819 | 1.0447 | 1.0138 | 0.9781 |
| 0.8669 | 0.9446 | 0.9880 | 0.9783 | 1.0484 | 0.9781 |
| 0.9004 | 0.9807 | 0.9880 | 0.8877 | 1.0426 | 1.0207 |
| 0.9116 | 0.9868 | 1.0783 | 0.9541 | 0.9562 | 1.0085 |
| 1.0179 | 1.0650 | 1.1024 | 1.0507 | 1.1290 | 1.0693 |
| 0.9508 | 0.9807 | 1.0060 | 0.9481 | 1.0138 | 1.1908 |
| 0.9508 | 1.0289 | 1.0060 | 0.9783 | 1.0484 | 1.0510 |
| 0.9060 | 0.9206 | 0.9277 | 0.9722 | 1.0253 | 0.9842 |
| 0.9676 | 0.9627 | 0.9096 | 1.0266 | 1.0311 | 0.9903 |
| 0.9116 | 0.8905 | 0.8795 | 0.8937 | 0.9505 | 0.9538 |
| 0.9564 | 0.9507 | 0.9458 | 0.8877 | 0.9389 | 0.9781 |
| 1.0212 | 1.0025 | 0.9615 | 1.0453 | 1.0313 | 1.0012 |
| 0.9527 | 0.9774 | 0.9505 | 0.9466 | 0.9881 | 0.9595 |
| 1.0710 | 1.0276 | 1.0495 | 1.0395 | 1.0583 | 0.9893 |
| 1.0710 | 1.0338 | 1.0330 | 1.1556 | 1.0745 | 0.9833 |
| 1.0710 | 1.0276 | 0.9945 | 1.1150 | 1.0205 | 0.9535 |
| 1.0648 | 1.0526 | 1.0165 | 1.1556 | 1.0529 | 1.0012 |
| 0.9776 | 0.9774 | 1.0165 | 1.0337 | 0.9665 | 0.9118 |
| 0.9776 | 0.9837 | 0.9890 | 1.0511 | 0.9233 | 0.9297 |
| 0.9402 | 0.9586 | 1.0055 | 1.0046 | 0.9341 | 0.8880 |
| 1.0149 | 0.9900 | 1.0330 | 1.1150 | 1.0205 | 0.9058 |
| 1.0399 | 0.9712 | 1.0165 | 0.8827 | 1.0853 | 0.8164 |
| 1.0025 | 0.9649 | 1.0110 | 1.0453 | 1.1015 | 0.9178 |
| 0.9963 | 0.9524 | 0.9945 | 0.9233 | 1.0259 | 0.8701 |
| 1.0772 | 1.0464 | 0.9780 | 0.9233 | 1.0313 | 0.8284 |
| 0.9340 | 0.9398 | 0.9835 | 0.9988 | 0.9395 | 0.9416 |
| 0.9589 | 1.0088 | 1.0330 | 1.0046 | 1.0097 | 0.8522 |
| 0.9340 | 0.9524 | 1.0165 | 0.9872 | 0.9233 | 0.8582 |
| 1.0087 | 1.0088 | 1.0714 | 0.9640 | 0.9935 | 0.8760 |
| 1.0090 | 1.0053 | 1.0244 | 0.9378 | 1.0409 | 0.9712 |
| 0.9190 | 0.9987 | 0.9022 | 1.0044 | 0.9386 | 0.8960 |
| 1.1054 | 1.0185 | 1.0303 | 0.9267 | 1.1131 | 1.0589 |
| 1.0733 | 0.9656 | 0.9837 | 0.9378 | 1.0590 | 0.9586 |
| 1.0604 | 0.9458 | 0.9895 | 0.9378 | 1.0890 | 0.9962 |
| 1.0411 | 0.9392 | 1.0070 | 0.8824 | 1.0229 | 0.9461 |
| 0.9447 | 0.9458 | 1.0012 | 0.9323 | 0.9988 | 0.9336 |
| 0.9319 | 0.9458 | 1.0361 | 0.9822 | 0.9928 | 0.8835 |
| 0.9126 | 0.9392 | 0.9837 | 0.9323 | 1.0289 | 0.8897 |
| 0.9769 | 1.0053 | 1.0419 | 0.9545 | 1.0289 | 1.0150 |
| 1.0411 | 0.9193 | 1.0536 | 0.8491 | 1.0710 | 0.9398 |
| 1.0990 | 0.9524 | 1.1176 | 0.9212 | 1.1312 | 0.9900 |
| 1.0347 | 0.9590 | 1.0768 | 0.8602 | 1.0770 | 0.9336 |
| 1.0540 | 0.9458 | 1.0885 | 0.8879 | 1.1071 | 0.9273 |
| 0.9447 | 0.9590 | 0.9837 | 0.8491 | 1.0229 | 0.9085 |
| 1.0476 | 0.9722 | 1.0361 | 0.8879 | 1.0710 | 0.9649 |
| 0.9769 | 0.9656 | 1.0070 | 0.8435 | 1.0229 | 0.9336 |
| 1.0347 | 0.9921 | 1.0303 | 0.8879 | 1.0409 | 0.9336 |

*Each row represents a different lot

Analytical outliers were identified using box-plot analysis on the normalized and log-transformed serum values (Table 15). Biological outliers were identified and excluded from analysis, and the normalized and log-transformed data for each individual serum sample was averaged across the six determinations, if available. Box-plot analysis was then performed to identify biological outliers (Table 16). Examples of the outliers analyzed are shown in Tables 15-17, for example. Tables 15, 16, 18, and 19 show examples of the analytical and biological outlier identification from the lots assessed in Tables 12-14. Table 17 illustrates an example of the summarized data of log-transformed rECL for S-CPF and T-CPF calculation after the removal of the biological and analytical outliers, such as those shown in Tables 15, 16, 18, and 19.

TABLE 15
Analytical and Biological Outlier Identification (Example A)

| Screening 1* | Screening 2 | Screening 3 | Screening 4 | Screening 5 | Screening 6 |
|---|---|---|---|---|---|
| 1.0179 | 0.9988 | 0.9880 | 1.0205 | 0.9793 | 0.9721 |
| 0.9452 | 1.0229 | 0.9398 | 1.3406 | 1.1463 | 1.0510 |
| 1.0011 | 1.0349 | 1.1325 | 1.0266 | 1.0023 | 1.1543 |
| 0.9899 | 1.0168 | 1.0843 | 1.0145 | 0.9735 | 1.0875 |
| 0.9060 | 0.9687 | 1.0361 | 0.9541 | 0.9620 | 1.0510 |
| 1.0403 | 1.0048 | 1.0241 | 1.0326 | 1.0023 | 1.0693 |
| 0.8389 | 0.9146 | 0.9880 | 0.9722 | 1.0023 | 0.9235 |
| 0.8669 | 0.9386 | 0.9819 | 1.0447 | 1.0138 | 0.9781 |
| 0.8669 | 0.9446 | 0.9880 | 0.9783 | 1.0484 | 0.9781 |
| 0.9004 | 0.9807 | 0.9880 | 0.8877 | 1.0426 | 1.0207 |
| 0.9116 | 0.9868 | 1.0783 | 0.9541 | 0.9562 | 1.0085 |
| 1.0179 | 1.0650 | 1.1024 | 1.0507 | 1.1290 | 1.0693 |
| 0.9508 | 0.9807 | 1.0060 | 0.9481 | 1.0138 | 1.1908 |
| 0.9508 | 1.0289 | 1.0060 | 0.9783 | 1.0484 | 1.0510 |
| 0.9060 | 0.9206 | 0.9277 | 0.9722 | 1.0253 | 0.9842 |
| 0.9676 | 0.9627 | 0.9096 | 1.0266 | 1.0311 | 0.9903 |
| 0.9116 | 0.8905 | 0.8795 | 0.8937 | 0.9505 | 0.9538 |
| 0.9564 | 0.9507 | 0.9458 | 0.8877 | 0.9389 | 0.9781 |
| 1.0212 | 1.0025 | 0.9615 | 1.0453 | 1.0313 | 1.0012 |
| 0.9527 | 0.9774 | 0.9505 | 0.9466 | 0.9881 | 0.9595 |
| 1.0710 | 1.0276 | 1.0495 | 1.0395 | 1.0583 | 0.9893 |
| 1.0710 | 1.0338 | 1.0330 | 1.1556 | 1.0745 | 0.9833 |
| 1.0710 | 1.0276 | 0.9945 | 1.1150 | 1.0205 | 0.9535 |
| 1.0648 | 1.0526 | 1.0165 | 1.1556 | 1.0529 | 1.0012 |
| 0.9776 | 0.9774 | 1.0165 | 1.0337 | 0.9665 | 0.9118 |
| 0.9776 | 0.9837 | 0.9890 | 1.0511 | 0.9233 | 0.9297 |
| 0.9402 | 0.9586 | 1.0055 | 1.0046 | 0.9341 | 0.8880 |
| 1.0149 | 0.9900 | 1.0330 | 1.1150 | 1.0205 | 0.9058 |
| 1.0399 | 0.9712 | 1.0165 | 0.8827 | 1.0853 | 0.8164 |
| 1.0025 | 0.9649 | 1.0110 | 1.0453 | 1.1015 | 0.9178 |
| 0.9963 | 0.9524 | 0.9945 | 0.9233 | 1.0259 | 0.8701 |
| 1.0772 | 1.0464 | 0.9780 | 0.9233 | 1.0313 | 0.8284 |
| 0.9340 | 0.9398 | 0.9835 | 0.9988 | 0.9395 | 0.9416 |
| 0.9589 | 1.0088 | 1.0330 | 1.0046 | 1.0097 | 0.8522 |
| 0.9340 | 0.9524 | 1.0165 | 0.9872 | 0.9233 | 0.8582 |
| 1.0087 | 1.0088 | 1.0714 | 0.9640 | 0.9935 | 0.8760 |
| 1.0090 | 1.0053 | 1.0244 | 0.9378 | 1.0409 | 0.9712 |
| 0.9190 | 0.9987 | 0.9022 | 1.0044 | 0.9386 | 0.8960 |
| 1.1054 | 1.0185 | 1.0303 | 0.9267 | 1.1131 | 1.0589 |
| 1.0733 | 0.9656 | 0.9837 | 0.9378 | 1.0590 | 0.9586 |
| 1.0604 | 0.9458 | 0.9895 | 0.9378 | 1.0890 | 0.9962 |
| 1.0411 | 0.9392 | 1.0070 | 0.8824 | 1.0229 | 0.9461 |
| 0.9447 | 0.9458 | 1.0012 | 0.9323 | 0.9988 | 0.9336 |
| 0.9319 | 0.9458 | 1.0361 | 0.9822 | 0.9928 | 0.8835 |
| 0.9126 | 0.9392 | 0.9837 | 0.9323 | 1.0289 | 0.8897 |
| 0.9769 | 1.0053 | 1.0419 | 0.9545 | 1.0289 | 1.0150 |
| 1.0411 | 0.9193 | 1.0536 | 0.8491 | 1.0710 | 0.9398 |
| 1.0990 | 0.9524 | 1.1176 | 0.9212 | 1.1312 | 0.9900 |
| 1.0347 | 0.9590 | 1.0768 | 0.8602 | 1.0770 | 0.9336 |
| 1.0540 | 0.9458 | 1.0885 | 0.8879 | 1.1071 | 0.9273 |
| 0.9447 | 0.9590 | 0.9837 | 0.8491 | 1.0229 | 0.9085 |
| 1.0476 | 0.9722 | 1.0361 | 0.8879 | 1.0710 | 0.9649 |
| 0.9769 | 0.9656 | 1.0070 | 0.8435 | 1.0229 | 0.9336 |
| 1.0347 | 0.9921 | 1.0303 | 0.8879 | 1.0409 | 0.9336 |

*Each row represents a different lot

TABLE 16
Analytical and Biological Outlier Identification (Example B)

| Screening 1* | Screening 2 | Screening 3 | Screening 4 | Screening 5 | Screening 6 | Mean |
|---|---|---|---|---|---|---|
| 1.0179 | 0.9988 | 0.9880 | 1.0205 | 0.9793 | 0.9721 | −0.0018 |
| 0.9452 | 1.0229 | 0.9398 | NA | 1.1463 | 1.0510 | 0.0078 |
| 1.0011 | 1.0349 | 1.1325 | 1.0266 | 1.0023 | 1.1543 | 0.0240 |
| 0.9899 | 1.0168 | 1.0843 | 1.0145 | 0.9735 | 1.0875 | 0.0115 |
| 0.9060 | 0.9687 | 1.0361 | 0.9541 | 0.9620 | 1.0510 | −0.0095 |
| 1.0403 | 1.0048 | 1.0241 | 1.0326 | 1.0023 | 1.0693 | 0.0123 |
| 0.8389 | 0.9146 | 0.9880 | 0.9722 | 1.0023 | 0.9235 | −0.0277 |
| 0.8669 | 0.9386 | 0.9819 | 1.0447 | 1.0138 | 0.9781 | −0.0137 |
| 0.8669 | 0.9446 | 0.9880 | 0.9783 | 1.0484 | 0.9781 | −0.0151 |
| 0.9004 | 0.9807 | 0.9880 | 0.8877 | 1.0426 | 1.0207 | −0.0140 |
| 0.9116 | 0.9868 | 1.0783 | 0.9541 | 0.9562 | 1.0085 | −0.0083 |
| 1.0179 | 1.0650 | 1.1024 | 1.0507 | 1.1290 | 1.0693 | 0.0301 |
| 0.9508 | 0.9807 | 1.0060 | 0.9481 | 1.0138 | 1.1908 | 0.0052 |
| 0.9508 | 1.0289 | 1.0060 | 0.9783 | 1.0484 | 1.0510 | 0.0043 |
| 0.9060 | 0.9206 | 0.9277 | 0.9722 | 1.0253 | 0.9842 | −0.0199 |
| 0.9676 | 0.9627 | 0.9096 | 1.0266 | 1.0311 | 0.9903 | −0.0086 |
| 0.9116 | 0.8905 | 0.8795 | 0.8937 | 0.9505 | 0.9538 | −0.0396 |
| 0.9564 | 0.9507 | 0.9458 | 0.8877 | 0.9389 | 0.9781 | −0.0257 |
| 1.0212 | 1.0025 | 0.9615 | 1.0453 | 1.0313 | 1.0012 | 0.0044 |
| 0.9527 | 0.9774 | 0.9505 | 0.9466 | 0.9881 | 0.9595 | −0.0167 |
| 1.0710 | 1.0276 | 1.0495 | 1.0395 | 1.0583 | 0.9893 | 0.0166 |
| 1.0710 | 1.0338 | 1.0330 | 1.1556 | 1.0745 | 0.9833 | 0.0242 |
| 1.0710 | 1.0276 | 0.9945 | 1.1150 | 1.0205 | 0.9535 | 0.0124 |
| 1.0648 | 1.0526 | 1.0165 | 1.1556 | 1.0529 | 1.0012 | 0.0237 |
| 0.9776 | 0.9774 | 1.0165 | 1.0337 | 0.9665 | 0.9118 | −0.0089 |
| 0.9776 | 0.9837 | 0.9890 | 1.0511 | 0.9233 | 0.9297 | −0.0111 |
| 0.9402 | 0.9586 | 1.0055 | 1.0046 | 0.9341 | 0.8880 | −0.0203 |
| 1.0149 | 0.9900 | 1.0330 | 1.1150 | 1.0205 | 0.9058 | 0.0049 |
| 1.0399 | 0.9712 | 1.0165 | 0.8827 | 1.0853 | 0.8164 | −0.0159 |
| 1.0025 | 0.9649 | 1.0110 | 1.0453 | 1.1015 | 0.9178 | 0.0024 |
| 0.9963 | 0.9524 | 0.9945 | 0.9233 | 1.0259 | 0.8701 | −0.0182 |
| 1.0772 | 1.0464 | 0.9780 | 0.9233 | 1.0313 | 0.8284 | −0.0101 |
| 0.9340 | 0.9398 | 0.9835 | 0.9988 | 0.9395 | 0.9416 | −0.0196 |
| 0.9589 | 1.0088 | 1.0330 | 1.0046 | 1.0097 | 0.8522 | −0.0106 |
| 0.9340 | 0.9524 | 1.0165 | 0.9872 | 0.9233 | 0.8582 | −0.0251 |
| 1.0087 | 1.0088 | 1.0714 | 0.9640 | 0.9935 | 0.8760 | −0.0064 |
| 1.0090 | 1.0053 | 1.0244 | 0.9378 | 1.0409 | 0.9712 | −0.0011 |
| 0.9190 | 0.9987 | 0.9022 | 1.0044 | 0.9386 | 0.8960 | −0.0259 |
| 1.1054 | 1.0185 | 1.0303 | 0.9267 | 1.1131 | 1.0589 | 0.0171 |
| 1.0733 | 0.9656 | 0.9837 | 0.9378 | 1.0590 | 0.9586 | −0.0022 |
| 1.0604 | 0.9458 | 0.9895 | 0.9378 | 1.0890 | 0.9962 | 0.0007 |
| 1.0411 | 0.9392 | 1.0070 | 0.8824 | 1.0229 | 0.9461 | −0.0126 |
| 0.9447 | 0.9458 | 1.0012 | 0.9323 | 0.9988 | 0.9336 | −0.0182 |
| 0.9319 | 0.9458 | 1.0361 | 0.9822 | 0.9928 | 0.8835 | −0.0174 |
| 0.9126 | 0.9392 | 0.9837 | 0.9323 | 1.0289 | 0.8897 | −0.0238 |
| 0.9769 | 1.0053 | 1.0419 | 0.9545 | 1.0289 | 1.0150 | 0.0015 |
| 1.0411 | 0.9193 | 1.0536 | 0.8491 | 1.0710 | 0.9398 | −0.0108 |
| 1.0990 | 0.9524 | 1.1176 | 0.9212 | 1.1312 | 0.9900 | 0.0136 |
| 1.0347 | 0.9590 | 1.0768 | 0.8602 | 1.0770 | 0.9336 | −0.0057 |
| 1.0540 | 0.9458 | 1.0885 | 0.8879 | 1.1071 | 0.9273 | −0.0008 |
| 0.9447 | 0.9590 | 0.9837 | 0.8491 | 1.0229 | 0.9085 | −0.0255 |
| 1.0476 | 0.9722 | 1.0361 | 0.8879 | 1.0710 | 0.9649 | −0.0023 |
| 0.9769 | 0.9656 | 1.0070 | 0.8435 | 1.0229 | 0.9336 | −0.0194 |
| 1.0347 | 0.9921 | 1.0303 | 0.8879 | 1.0409 | 0.9336 | −0.0066 |

*Each row represents a different lot

TABLE 17
log-transformed rECL for S-CPF and T-CPF

| Screening 1* | Screening 2 | Screening 3 | Screening 4 | Screening 5 | Screening 6 |
|---|---|---|---|---|---|
| 1.0179 | 0.9988 | 0.9880 | 1.0205 | 0.9793 | 0.9721 |
| 0.9452 | 1.0229 | 0.9398 | NA | 1.1463 | 1.0510 |
| 1.0011 | 1.0349 | 1.1325 | 1.0266 | 1.0023 | 1.1543 |
| 0.9899 | 1.0168 | 1.0843 | 1.0145 | 0.9735 | 1.0875 |
| 0.9060 | 0.9687 | 1.0361 | 0.9541 | 0.9620 | 1.0510 |
| 1.0403 | 1.0048 | 1.0241 | 1.0326 | 1.0023 | 1.0693 |
| 0.8389 | 0.9146 | 0.9880 | 0.9722 | 1.0023 | 0.9235 |
| 0.8669 | 0.9386 | 0.9819 | 1.0447 | 1.0138 | 0.9781 |
| 0.8669 | 0.9446 | 0.9880 | 0.9783 | 1.0484 | 0.9781 |
| 0.9004 | 0.9807 | 0.9880 | 0.8877 | 1.0426 | 1.0207 |
| 0.9116 | 0.9868 | 1.0783 | 0.9541 | 0.9562 | 1.0085 |
| 1.0179 | 1.0650 | 1.1024 | 1.0507 | 1.1290 | 1.0693 |
| 0.9508 | 0.9807 | 1.0060 | 0.9481 | 1.0138 | 1.1908 |
| 0.9508 | 1.0289 | 1.0060 | 0.9783 | 1.0484 | 1.0510 |
| 0.9060 | 0.9206 | 0.9277 | 0.9722 | 1.0253 | 0.9842 |
| 0.9676 | 0.9627 | 0.9096 | 1.0266 | 1.0311 | 0.9903 |
| 0.9116 | 0.8905 | 0.8795 | 0.8937 | 0.9505 | 0.9538 |
| 0.9564 | 0.9507 | 0.9458 | 0.8877 | 0.9389 | 0.9781 |
| 1.0212 | 1.0025 | 0.9615 | 1.0453 | 1.0313 | 1.0012 |
| 0.9527 | 0.9774 | 0.9505 | 0.9466 | 0.9881 | 0.9595 |
| 1.0710 | 1.0276 | 1.0495 | 1.0395 | 1.0583 | 0.9893 |
| 1.0710 | 1.0338 | 1.0330 | 1.1556 | 1.0745 | 0.9833 |
| 1.0710 | 1.0276 | 0.9945 | 1.1150 | 1.0205 | 0.9535 |
| 1.0648 | 1.0526 | 1.0165 | 1.1556 | 1.0529 | 1.0012 |
| 0.9776 | 0.9774 | 1.0165 | 1.0337 | 0.9665 | 0.9118 |
| 0.9776 | 0.9837 | 0.9890 | 1.0511 | 0.9233 | 0.9297 |
| 0.9402 | 0.9586 | 1.0055 | 1.0046 | 0.9341 | 0.8880 |
| 1.0149 | 0.9900 | 1.0330 | 1.1150 | 1.0205 | 0.9058 |
| 1.0399 | 0.9712 | 1.0165 | 0.8827 | 1.0853 | 0.8164 |
| 1.0025 | 0.9649 | 1.0110 | 1.0453 | 1.1015 | 0.9178 |
| 0.9963 | 0.9524 | 0.9945 | 0.9233 | 1.0259 | 0.8701 |
| 1.0772 | 1.0464 | 0.9780 | 0.9233 | 1.0313 | 0.8284 |
| 0.9340 | 0.9398 | 0.9835 | 0.9988 | 0.9395 | 0.9416 |
| 0.9589 | 1.0088 | 1.0330 | 1.0046 | 1.0097 | 0.8522 |
| 0.9340 | 0.9524 | 1.0165 | 0.9872 | 0.9233 | 0.8582 |
| 1.0087 | 1.0088 | 1.0714 | 0.9640 | 0.9935 | 0.8760 |
| 1.0090 | 1.0053 | 1.0244 | 0.9378 | 1.0409 | 0.9712 |
| 0.9190 | 0.9987 | 0.9022 | 1.0044 | 0.9386 | 0.8960 |
| 1.1054 | 1.0185 | 1.0303 | 0.9267 | 1.1131 | 1.0589 |
| 1.0733 | 0.9656 | 0.9837 | 0.9378 | 1.0590 | 0.9586 |
| 1.0604 | 0.9458 | 0.9895 | 0.9378 | 1.0890 | 0.9962 |
| 1.0411 | 0.9392 | 1.0070 | 0.8824 | 1.0229 | 0.9461 |
| 0.9447 | 0.9458 | 1.0012 | 0.9323 | 0.9988 | 0.9336 |
| 0.9319 | 0.9458 | 1.0361 | 0.9822 | 0.9928 | 0.8835 |
| 0.9126 | 0.9392 | 0.9837 | 0.9323 | 1.0289 | 0.8897 |
| 0.9769 | 1.0053 | 1.0419 | 0.9545 | 1.0289 | 1.0150 |
| 1.0411 | 0.9193 | 1.0536 | 0.8491 | 1.0710 | 0.9398 |
| 1.0990 | 0.9524 | 1.1176 | 0.9212 | 1.1312 | 0.9900 |
| 1.0347 | 0.9590 | 1.0768 | 0.8602 | 1.0770 | 0.9336 |
| 1.0540 | 0.9458 | 1.0885 | 0.8879 | 1.1071 | 0.9273 |
| 0.9447 | 0.9590 | 0.9837 | 0.8491 | 1.0229 | 0.9085 |
| 1.0476 | 0.9722 | 1.0361 | 0.8879 | 1.0710 | 0.9649 |
| 0.9769 | 0.9656 | 1.0070 | 0.8435 | 1.0229 | 0.9336 |
| 1.0347 | 0.9921 | 1.0303 | 0.8879 | 1.0409 | 0.9336 |

*Each row represents a different lot

TABLE 18
Calculation of SCP and TCP Analytical Outliers

| Measurement | Value |
|---|---|
| Q1 (25% quartile) | −0.0243 |
| Q3 (75% quartile) | 0.0135 |
| Q3 − Q1 | 0.0378 |
| High end (Q3 + 3*[Q3 − Q1]) | 0.1269 |
| Low end (Q1 − 3*[Q3 − Q1]) | −0.1377 |

TABLE 19
Calculation of SCP and TCP Biological Outliers

| Measurement | Value |
|---|---|
| Q1 (25% quartile) | −0.0172 |
| Q3 (75% quartile) | 0.0044 |
| Q3 − Q1 | 0.0216 |
| High end (Q3 + 3*[Q3 − Q1]) | 0.0692 |
| Low end (Q1 − 3*[Q3 − Q1]) | −0.0820 |

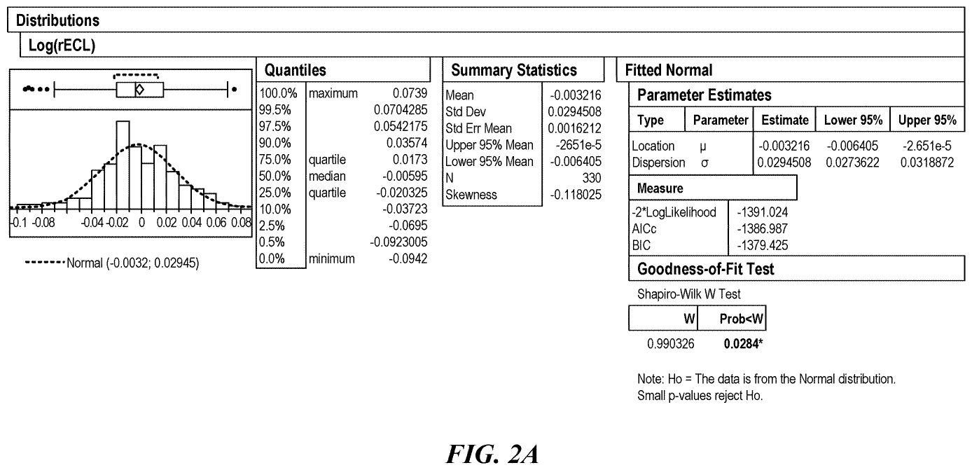

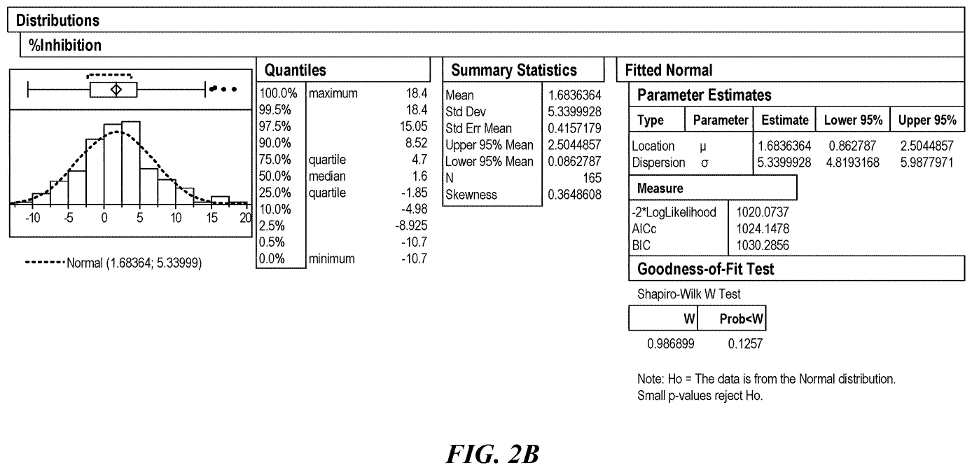

Screening and Titration Data Normality Test: The normality of 323 log-transformed normalized values were tested by Shapiro-Wilk W Test using JMP software (FIG. 2A). Normal distribution was confirmed, and a parametric approach was used to calculate S-CPF and T-CPF (Table 20).

TABLE 20
Calculated Parametric S-CPF and T-CPF

| Measurement | Value |
|---|---|
| Mean | −0.0056 |
| SD | 0.0283 |
| Log(S-CPF) | 0.04 |
| S-CPF | 1.10 |
| Log(T-CPF) | 0.08 |
| T-CPF | 1.20 |

CCP Determination: For each run, % inhibition for each sample was calculated using the following formula:

$$\% inhibition = 100 * ( {ECL}_{without drug} - {ECL}_{with drug} ) / {ECL}_{without drug}$$

SCPs and TCPs were removed from the CCP analysis.

CCP analytical outliers were identified and excluded from analysis using box-plot analysis on the % inhibition values (Table 21).

TABLE 21
Calculation of CCP Analytical Outliers

| Measurement | Value |
|---|---|
| Q1 (25% quartile) | 0.5600 |
| Q3 (75% quartile) | 8.2100 |
| Q3 − Q1 | 7.6500 |
| High end (Q3 + 3*[Q3 − Q1]) | 31.1600 |
| Low end (Q1 − 3*[Q3 − Q1]) | −22.3900 |

After identification and exclusion of analytical outliers, % inhibition data for each individual serum sample was averaged across the six determinations, if available. Box-plot analysis was performed on the values to identify biological outliers (Table 22).

TABLE 22
Calculation of CCP Biological Outliers

| Measurement | Value |
|---|---|
| Q1 (25% quartile) | 1.5850 |
| Q3 (75% quartile) | 6.1625 |
| Q3 − Q1 | 4.5775 |
| High end (Q3 + 3*[Q3 − Q1]) | 19.8950 |
| Low end (Q1 − 3*[Q3 − Q1]) | −12.1475 |

Confirmatory Data Normality Test: Normality of 323 values of % inhibition were tested by Shapiro-Wilk W Test using JMP software (FIG. 2B). Normal distribution was confirmed, and a parametric approach was used to calculate S-CPF and T-CPF (Table 23).

TABLE 23
Calculated Parametric CCP Factors

| Measurement | Value |
|---|---|
| Mean | 3.8557 |
| SD | 6.1523 |
| CCP (%) | 16.45 |

Cut-point values were determined for application of the assay to detect ADAs to XTMAB-16 in human serum (Table 24). Examples of the CCP values assessed are shown in Tables 25-28. An example of the raw data values assessed is shown in Table 25, and examples of these values after the removal of outliers are shown in Tables 26-28, no outliers were found in the example.

TABLE 24
Cut-Point Values Determined for Detection Assay

| | | False-Positive | Parametric/ | |
|---|---|---|---|---|
| Description | Units | Rate | Non-Parametric | Result |
| S-CPF | NA | 5% | Non-Parametric | 1.10 |
| T-CPF | NA | 0.1% | Non-Parametric | 1.20 |
| CCP | % | 1% | Non-Parametric | 16.45 |

TABLE 25
Exemplary Raw Data

| Example | Drug Unspiked | | | | Drug Spiked (MAB-16 at 200 ug/mL) | | | | |
|---|---|---|---|---|---|---|---|---|---|
| Run* | Rep 1 | Rep 2 | Mean | % CV | Rep 1 | Rep 2 | Mean | % CV | % Inhibition |
| 34 | 94 | 88 | 91.0 | 4.7 | 82 | 80 | 81.0 | 1.7 | 10.99 |
| | 86 | 83 | 84.5 | 2.5 | 92 | 83 | 87.5 | 7.3 | −3.55 |
| | 93 | 86 | 89.5 | 5.5 | 79 | 87 | 83.0 | 6.8 | 7.26 |
| | 91 | 86 | 88.5 | 4.0 | 87 | 86 | 86.5 | 0.8 | 2.26 |
| | 82 | 80 | 81.0 | 1.7 | 82 | 77 | 79.5 | 4.4 | 1.85 |
| | 95 | 91 | 93.0 | 3.0 | 96 | 88 | 92.0 | 6.1 | 1.08 |
| | 77 | 73 | 75.0 | 3.8 | 73 | 68 | 70.5 | 5.0 | 6.00 |
| | 79 | 76 | 77.5 | 2.7 | 83 | 76 | 79.5 | 6.2 | −2.58 |
| | 79 | 76 | 77.5 | 2.7 | 82 | 71 | 76.5 | 10.2 | 1.29 |
| | 79 | 82 | 80.5 | 2.6 | 82 | 77 | 79.5 | 4.4 | 1.24 |
| | 78 | 85 | 81.5 | 6.1 | 92 | 88 | 90.0 | 3.1 | −10.43 |
| | 92 | 90 | 91.0 | 1.6 | 92 | 87 | 89.5 | 4.0 | 1.65 |
| | 89 | 81 | 85.0 | 6.7 | 84 | 82 | 83.0 | 1.7 | 2.35 |
| | 88 | 82 | 85.0 | 5.0 | 83 | 78 | 80.5 | 4.4 | 5.29 |
| | 81 | 81 | 81.0 | 0.0 | 79 | 73 | 76.0 | 5.6 | 6.17 |
| | 89 | 84 | 86.5 | 4.1 | 78 | 77 | 77.5 | 0.9 | 10.40 |
| | 83 | 80 | 81.5 | 2.6 | 77 | 73 | 75.0 | 3.8 | 7.98 |
| | 87 | 84 | 85.5 | 2.5 | 78 | 76 | 77.0 | 1.8 | 9.94 |
| 35 | 81 | 83 | 82.0 | 1.7 | 81 | 82 | 81.5 | 0.9 | 0.61 |
| | 76 | 77 | 76.5 | 0.9 | 77 | 76 | 76.5 | 0.9 | 0.00 |
| | 84 | 88 | 86.0 | 3.3 | 72 | 72 | 720 | 0.0 | 16.28 |
| | 85 | 87 | 86.0 | 1.6 | 74 | 72 | 73.0 | 1.9 | 15.12 |
| | 83 | 89 | 86.0 | 4.9 | 78 | 70 | 74.0 | 7.6 | 13.95 |
| | 83 | 88 | 85.5 | 4.1 | 77 | 75 | 76.0 | 1.9 | 11.11 |
| | 80 | 77 | 78.5 | 2.7 | 70 | 72 | 710 | 2.0 | 9.55 |
| | 80 | 77 | 78.5 | 2.7 | 72 | 72 | 72.0 | 0.0 | 8.28 |
| | 78 | 73 | 75.5 | 4.7 | 72 | 69 | 70.5 | 3.0 | 6.62 |
| | 81 | 82 | 81.5 | 0.9 | 77 | 80 | 78.5 | 2.7 | 3.68 |
| | 80 | 87 | 83.5 | 5.9 | 75 | 85 | 80.0 | 8.8 | 4.19 |
| | 79 | 82 | 80.5 | 2.6 | 69 | 73 | 71.0 | 4.0 | 11.80 |
| | 78 | 82 | 80.0 | 3.5 | 68 | 73 | 70.5 | 5.0 | 11.88 |
| | 85 | 88 | 86.5 | 2.5 | 74 | 78 | 76.0 | 3.7 | 12.14 |
| | 76 | 74 | 75.0 | 1.9 | 71 | 80 | 75.5 | 8.4 | −0.67 |
| | 79 | 75 | 77.0 | 3.7 | 76 | 85 | 80.5 | 7.9 | −4.55 |
| | 75 | 75 | 75.0 | 0.0 | 69 | 76 | 725 | 6.8 | 3.33 |
| | 81 | 81 | 81.0 | 0.0 | 78 | 80 | 79.0 | 1.8 | 2.47 |
| 36 | 78 | 79 | 78.5 | 0.9 | 85 | 79 | 82.0 | 5.2 | −4.46 |
| | 67 | 76 | 71.5 | 8.9 | 84 | 76 | 80.0 | 7.1 | −11.89 |
| | 88 | 84 | 86.0 | 3.3 | 73 | 74 | 73.5 | 1.0 | 14.53 |
| | 86 | 81 | 83.5 | 4.2 | 76 | 75 | 75.5 | 0.9 | 9.58 |

*Each row represents a different lot

TABLE 26
Values after Outlier Removal (Example A)

| Confirmatory 1 | Confirmatory 2 | Confirmatory 3 | Confirmatory 4 | Confirmatory 5 | Confirmatory 6 |
|---|---|---|---|---|---|
| 11.0 | −1.8 | −5.5 | −3.0 | 2.4 | −2.5 |
| −3.6 | −5.9 | −9.0 | NA | −5.0 | −11.6 |
| 7.3 | 11.1 | 16.5 | 10.0 | 3.5 | 16.3 |
| 2.3 | 8.3 | 13.9 | 5.4 | 0.6 | 7.3 |
| 1.9 | 6.8 | 11.1 | 8.2 | 6.0 | 9.8 |
| 1.1 | 6.6 | 11.8 | 8.2 | 0.0 | 7.4 |
| 6.0 | 4.0 | 6.1 | 6.2 | 9.2 | 5.9 |
| −2.6 | 0.0 | 4.3 | 1.2 | 4.6 | 5.6 |
| 1.3 | 6.4 | 4.9 | −0.6 | 4.4 | 5.6 |
| 1.2 | 1.2 | −6.7 | −3.4 | 8.8 | 7.1 |
| −10.4 | −6.1 | 0.0 | −3.2 | −1.2 | −10.2 |
| 1.7 | 6.2 | 7.1 | 6.9 | 9.7 | 3.4 |
| 2.4 | 3.7 | 1.8 | 4.5 | 5.1 | 16.3 |
| 5.3 | 6.4 | 3.6 | 6.2 | 5.5 | 2.9 |
| 6.2 | 2.0 | −12.3 | 4.4 | 5.1 | −5.6 |
| 10.4 | 4.4 | −9.9 | 5.9 | −0.6 | −4.3 |
| 8.0 | 0.7 | −10.3 | 1.4 | 3.0 | −1.9 |
| 9.9 | −3.2 | −7.0 | −2.0 | −7.4 | −7.5 |
| 0.6 | −3.8 | 0.6 | −5.0 | −6.8 | 1.2 |
| 0.0 | −1.3 | −2.9 | −10.4 | 6.0 | 6.8 |
| 16.3 | 6.1 | 9.4 | 18.4 | 8.7 | 3.0 |
| 15.1 | 6.1 | 0.5 | 9.1 | −2.0 | −0.6 |
| 14.0 | 11.6 | 5.0 | 13.0 | 5.8 | 7.5 |
| 11.1 | 8.9 | 2.7 | 4.5 | 3.1 | 7.7 |
| 9.6 | 7.7 | 10.8 | −2.3 | 2.2 | 7.8 |
| 8.3 | 1.9 | 3.9 | 1.1 | −1.8 | 3.2 |
| 6.6 | 8.5 | 8.7 | 4.6 | −0.6 | 2.0 |
| 3.7 | 5.7 | 4.8 | 4.2 | 2.7 | −2.6 |
| 4.2 | −6.5 | −1.1 | −13.2 | 9.5 | 1.5 |
| 11.8 | 3.3 | 0.5 | 1.1 | 9.3 | 5.8 |
| 11.9 | 1.3 | 6.6 | −4.4 | 5.8 | 9.6 |
| 12.1 | 6.0 | 0.6 | −3.8 | 6.8 | 15.1 |
| −0.7 | 8.7 | 10.6 | −5.2 | 6.3 | 7.0 |
| −4.6 | 6.2 | 11.2 | 0.0 | 4.3 | 16.8 |
| 3.3 | 2.0 | 13.0 | −2.4 | 3.5 | 9.0 |
| 2.5 | 5.0 | 10.8 | −7.8 | −0.5 | 10.2 |
| −4.5 | 9.9 | −1.7 | 8.3 | −11.0 | 2.6 |
| −11.9 | 9.3 | −6.5 | 1.7 | −9.0 | −7.0 |
| 14.5 | 7.1 | −1.7 | 9.6 | 4.9 | 8.9 |
| 9.6 | 4.1 | −4.7 | 7.1 | 5.1 | 3.9 |
| 10.9 | 1.4 | −4.7 | 10.1 | 8.8 | 5.7 |
| 12.4 | 0.0 | −4.1 | 8.8 | 0.6 | 7.3 |
| 10.9 | 4.2 | 9.3 | 16.7 | 9.0 | 10.7 |
| 2.8 | −0.7 | 6.7 | 14.1 | 3.0 | 0.0 |
| 1.4 | 1.4 | 4.1 | 10.7 | 11.7 | 5.6 |
| 1.3 | 2.0 | 0.6 | 9.9 | 5.3 | 7.4 |
| −0.6 | −7.9 | −0.6 | −10.5 | −2.3 | −10.7 |

*Each row represents a different lot

TABLE 27
Values after Outlier Removal (Example B)

| Confirmatory 1* | Confirmatory 2 | Confirmatory 3 | Confirmatory 4 | Confirmatory 5 | Confirmatory 6 |
|---|---|---|---|---|---|
| 11.0 | −1.8 | −5.5 | −3.0 | 2.4 | −2.5 |
| −3.6 | −5.9 | −9.0 | NA | −5.0 | −11.6 |
| 7.3 | 11.1 | 16.5 | 10.0 | 3.5 | 16.3 |
| 2.3 | 8.3 | 13.9 | 5.4 | 0.6 | 7.3 |
| 1.9 | 6.8 | 11.1 | 8.2 | 6.0 | 9.8 |
| 1.1 | 6.6 | 11.8 | 8.2 | 0.0 | 7.4 |
| 6.0 | 4.0 | 6.1 | 6.2 | 9.2 | 5.9 |
| −2.6 | 0.0 | 4.3 | 1.2 | 4.6 | 5.6 |
| 1.3 | 6.4 | 4.9 | −0.6 | 4.4 | 5.6 |
| 1.2 | 1.2 | −6.7 | −3.4 | 8.8 | 7.1 |
| −10.4 | −6.1 | 0.0 | −3.2 | −1.2 | −10.2 |
| 1.7 | 6.2 | 7.1 | 6.9 | 9.7 | 3.4 |
| 2.4 | 3.7 | 1.8 | 4.5 | 5.1 | 16.3 |
| 5.3 | 6.4 | 3.6 | 6.2 | 5.5 | 2.9 |
| 6.2 | 2.0 | −12.3 | 4.4 | 5.1 | −5.6 |
| 10.4 | 4.4 | −9.9 | 5.9 | −0.6 | −4.3 |
| 8.0 | 0.7 | −10.3 | 1.4 | 3.0 | −1.9 |
| 9.9 | −3.2 | −7.0 | −2.0 | −7.4 | −7.5 |
| 0.6 | −3.8 | 0.6 | −5.0 | −6.8 | 1.2 |
| 0.0 | −1.3 | −2.9 | −10.4 | 6.0 | 6.8 |
| 16.3 | 6.1 | 9.4 | 18.4 | 8.7 | 3.0 |
| 15.1 | 6.1 | 0.5 | 9.1 | −2.0 | −0.6 |
| 14.0 | 11.6 | 5.0 | 13.0 | 5.8 | 7.5 |
| 11.1 | 8.9 | 2.7 | 4.5 | 3.1 | 7.7 |
| 9.6 | 7.7 | 10.8 | −2.3 | 2.2 | 7.8 |
| 8.3 | 1.9 | 3.9 | 1.1 | −1.8 | 3.2 |
| 6.6 | 8.5 | 8.7 | 4.6 | −0.6 | 2.0 |
| 3.7 | 5.7 | 4.8 | 4.2 | 2.7 | −2.6 |
| 4.2 | −6.5 | −1.1 | −13.2 | 9.5 | 1.5 |
| 11.8 | 3.3 | 0.5 | 1.1 | 9.3 | 5.8 |
| 11.9 | 1.3 | 6.6 | −4.4 | 5.8 | 9.6 |
| 12.1 | 6.0 | 0.6 | −3.8 | 6.8 | 15.1 |
| −0.7 | 8.7 | 10.6 | −5.2 | 6.3 | 7.0 |
| −4.6 | 6.2 | 11.2 | 0.0 | 4.3 | 16.8 |
| 3.3 | 2.0 | 13.0 | −2.4 | 3.5 | 9.0 |
| 2.5 | 5.0 | 10.8 | −7.8 | −0.5 | 10.2 |
| −4.5 | 9.9 | −1.7 | 8.3 | −11.0 | 2.6 |
| −11.9 | 9.3 | −6.5 | 1.7 | −9.0 | −7.0 |
| 14.5 | 7.1 | −1.7 | 9.6 | 4.9 | 8.9 |
| 9.6 | 4.1 | −4.7 | 7.1 | 5.1 | 3.9 |
| 10.9 | 1.4 | −4.7 | 10.1 | 8.8 | 5.7 |
| 12.4 | 0.0 | −4.1 | 8.8 | 0.6 | 7.3 |
| 10.9 | 4.2 | 9.3 | 16.7 | 9.0 | 10.7 |
| 2.8 | −0.7 | 6.7 | 14.1 | 3.0 | 0.0 |
| 1.4 | 1.4 | 4.1 | 10.7 | 11.7 | 5.6 |
| 1.3 | 2.0 | 0.6 | 9.9 | 5.3 | 7.4 |
| −0.6 | −7.9 | −0.6 | −10.5 | −2.3 | −10.7 |
| 9.4 | 4.2 | 5.2 | 4.8 | 11.7 | 3.2 |
| 6.2 | 8.3 | 2.7 | 1.3 | 14.0 | 4.7 |
| 9.2 | 7.0 | 4.3 | 2.5 | 11.4 | 2.7 |
| 6.1 | 0.7 | 0.6 | 3.9 | 6.5 | 5.5 |
| 16.0 | 3.4 | 5.1 | 6.3 | 9.6 | 6.5 |
| 13.2 | 6.9 | 7.5 | 1.3 | 10.6 | 10.7 |
| 14.9 | 2.0 | 2.3 | 2.5 | 6.4 | 5.4 |

*Each row represents a different lot

TABLE 28
Values after Outlier Removal (Example C)

| Confirmatory 1* | Confirmatory 2 | Confirmatory 3 | Confirmatory 4 | Confirmatory 5 | Confirmatory 6 |
|---|---|---|---|---|---|
| 11.0 | −1.8 | −5.5 | −3.0 | 2.4 | −2.5 |
| −3.6 | −5.9 | −9.0 | NA | −5.0 | −11.6 |
| 7.3 | 11.1 | 16.5 | 10.0 | 3.5 | 16.3 |
| 2.3 | 8.3 | 13.9 | 5.4 | 0.6 | 7.3 |
| 1.9 | 6.8 | 11.1 | 8.2 | 6.0 | 9.8 |
| 1.1 | 6.6 | 11.8 | 8.2 | 0.0 | 7.4 |
| 6.0 | 4.0 | 6.1 | 6.2 | 9.2 | 5.9 |
| −2.6 | 0.0 | 4.3 | 1.2 | 4.6 | 5.6 |
| 1.3 | 6.4 | 4.9 | −0.6 | 4.4 | 5.6 |
| 1.2 | 1.2 | −6.7 | −3.4 | 8.8 | 7.1 |
| −10.4 | −6.1 | 0.0 | −3.2 | −1.2 | −10.2 |
| 1.7 | 6.2 | 7.1 | 6.9 | 9.7 | 3.4 |
| 2.4 | 3.7 | 1.8 | 4.5 | 5.1 | 16.3 |
| 5.3 | 6.4 | 3.6 | 6.2 | 5.5 | 2.9 |
| 6.2 | 2.0 | −12.3 | 4.4 | 5.1 | −5.6 |
| 10.4 | 4.4 | −9.9 | 5.9 | −0.6 | −4.3 |
| 8.0 | 0.7 | −10.3 | 1.4 | 3.0 | −1.9 |
| 9.9 | −3.2 | −7.0 | −2.0 | −7.4 | −7.5 |
| 0.6 | −3.8 | 0.6 | −5.0 | −6.8 | 1.2 |
| 0.0 | −1.3 | −2.9 | −10.4 | 6.0 | 6.8 |
| 16.3 | 6.1 | 9.4 | 18.4 | 8.7 | 3.0 |
| 15.1 | 6.1 | 0.5 | 9.1 | −2.0 | −0.6 |
| 14.0 | 11.6 | 5.0 | 13.0 | 5.8 | 7.5 |
| 11.1 | 8.9 | 2.7 | 4.5 | 3.1 | 7.7 |
| 9.6 | 7.7 | 10.8 | −2.3 | 2.2 | 7.8 |
| 8.3 | 1.9 | 3.9 | 1.1 | −1.8 | 3.2 |
| 6.6 | 8.5 | 8.7 | 4.6 | −0.6 | 2.0 |
| 3.7 | 5.7 | 4.8 | 4.2 | 2.7 | −2.6 |
| 4.2 | −6.5 | −1.1 | −13.2 | 9.5 | 1.5 |
| 11.8 | 3.3 | 0.5 | 1.1 | 9.3 | 5.8 |
| 11.9 | 1.3 | 6.6 | −4.4 | 5.8 | 9.6 |
| 12. | 6.0 | 0.6 | −3.8 | 6.8 | 15.1 |
| −0.7 | 8.7 | 10.6 | −5.2 | 6.3 | 7.0 |
| −4.6 | 6.2 | 11.2 | 0.0 | 4.3 | 16.8 |
| 3.3 | 2.0 | 13.0 | −2.4 | 3.5 | 9.0 |
| 2.5 | 5.0 | 10.8 | −7.8 | −0.5 | 10.2 |
| −4.5 | 9.9 | −1.7 | 8.3 | −11.0 | 2.6 |
| −11.9 | 9.3 | −6.5 | 1.7 | −9.0 | −7.0 |
| 14.5 | 7.1 | −1.7 | 9.6 | 4.9 | 8.9 |
| 9.6 | 4.1 | −4.7 | 7.1 | 5.1 | 3.9 |
| 10.9 | 1.4 | −4.7 | 10.1 | 8.8 | 5.7 |
| 12.4 | 0.0 | −4.1 | 8.8 | 0.6 | 7.3 |
| 10.9 | 4.2 | 9.3 | 16.7 | 9.0 | 10.7 |
| 2.8 | −0.7 | 6.7 | 14.1 | 3.0 | 0.0 |
| 1.4 | 1.4 | 4.1 | 10.7 | 11.7 | 5.6 |
| 1.3 | 2.0 | 0.6 | 9.9 | 5.3 | 7.4 |
| −0.6 | −7.9 | −0.6 | −10.5 | −2.3 | −10.7 |
| 9.4 | 4.2 | 5.2 | 4.8 | 11.7 | 3.2 |
| 6.2 | 8.3 | 2.7 | 1.3 | 14.0 | 4.7 |
| 9.2 | 7.0 | 4.3 | 2.5 | 11.4 | 2.7 |
| 6.1 | 0.7 | 0.6 | 3.9 | 6.5 | 5.5 |
| 16.0 | 3.4 | 5.1 | 6.3 | 9.6 | 6.5 |
| 13.2 | 6.9 | 7.5 | 1.3 | 10.6 | 10.7 |
| 14.9 | 2.0 | 2.3 | 2.5 | 6.4 | 5.4 |

*Each row represents a different lot

Sensitivity: The screening assay sensitivity was evaluated six times, in pooled human serum (three times) and three individual lots of human serum (one time for each lot). Each sample was spiked with anti-XTMAB-16 antibodies PC. Serial two-fold dilutions of the spiked samples (from 4,000 ng/ml to 0.49 ng/ml) in pNHS and individual mouse serum (iNMS) were performed. The screening sensitivity was determined for each sensitivity set using Microsoft Excel Forecast, by interpolation at the SCP (5% false-positive). The final assay sensitivity in screening assay is the mean of calculated concentration among all the qualified curves. The results showed that the screening sensitivity of the assay to detect anti-XTMAB-16 antibodies was 2.77 ng/mL in human serum. An example of this result is shown in Table 29.

TABLE 29
Screening Sensitivity

| | | Screening Assay | | | | | | | | | | | | | | |
|---|---|---|---|---|---|---|---|---|---|---|---|---|---|---|---|---|
| | | Sensitivity in Individual serum | | | | | | | | | | | | | | |
| | | Run Example | | | | | | | | | | | | | | |
| | | Sensitivity 01 | | | | | Sensitivity 02 | | | | | Sensitivity 03 | | | | |
| | ADA | (Individual-01) Run 55 | | | | | (Individual-02) Run 56 | | | | | (Individual-03) Run 57 | | | | |
| | Cone. | | | Plate | Plate | Plate | | | Plate | Plate | Plate | | | Plate | Plate | Plate |
| Titer | (ng/mL) | ECL | % CV | NC | SCP | TCP | ECL | % CV | NC | SCP | TCP | ECL | % CV | NC | SCP | TCP |
| 1 | 4000.00 | 7790.5 | 2.5 | 82.8 | 91.08 | 99.36 | 8550.0 | 0.3 | 81.8 | 89.98 | 98.16 | 8489.5 | 1.6 | 88.9 | 97.79 | 106.68 |
| 2 | 2000.00 | 6952.0 | 3.5 | | | | 7490.0 | 1.1 | | | | 7082.0 | 10.3 | | | |
| 4 | 1000.00 | 4194.0 | 0.9 | | | | 4613.5 | 1.0 | | | | 4428.5 | 9.2 | | | |
| 8 | 500.00 | 1520.0 | 1.0 | | | | 1653.5 | 0.7 | | | | 1703.0 | 1.0 | | | |
| 16 | 250.00 | 742.5 | 2.2 | | | | 789.5 | 0.6 | | | | 785.0 | 5.0 | | | |
| 32 | 125.00 | 375.5 | 2.1 | | | | 377.5 | 0.9 | | | | 489.0 | 3.2 | | | |
| 64 | 62.50 | 200.0 | 9.2 | | | | 248.5 | 3.1 | | | | 283.0 | 1.5 | | | |
| 128 | 31.25 | 156.5 | 1.4 | | | | 163.5 | 0.4 | | | | 185.0 | 1.5 | | | |
| 256 | 15.63 | 122.5 | 4.0 | | | | 116.5 | 0.6 | | | | 130.5 | 7.0 | | | |
| 512 | 7.81 | 110.0 | 3.9 | | | | 102.0 | 2.8 | | | | 110.5 | 1.9 | | | |
| 1024 | 3.91 | 94.0 | 1.5 | | | | 88.0 | 4.8 | | | | 97.5 | 0.7 | | | |
| 2048 | 1.95 | 89.5 | 0.8 | | | | 84.0 | 1.7 | | | | 90.0 | 0.0 | | | |
| 4096 | 0.98 | 83.0 | 8.5 | | | | 79.0 | 1.8 | | | | 86.5 | 4.1 | | | |
| 8192 | 0.49 | 82.5 | 6.0 | | | | 82.5 | 0.9 | | | | 87.0 | 3.3 | | | |

Confirmatory assay sensitivity was performed by spiking XTMAB-16 into diluted samples prepared for screening sensitivity. The confirmatory sensitivity is determined for each sensitivity set using Microsoft Excel Forecast, by interpolation at the CCP (1% false-positive). The final assay sensitivity in confirmatory assay is the mean of calculated concentration among all the qualified curves. The results showed that the confirmatory sensitivity of the assay to detect anti-XTMAB-16 antibodies was 4.25 ng/mL. An example of this result is shown in Table 30.

TABLE 30
Confirmatory Sensitivity

| | | Confirmatory Sensitivity | | | | | | | | | | | |
|---|---|---|---|---|---|---|---|---|---|---|---|---|---|
| | | Sensitivity in Individual serum | | | | | | | | | | | |
| | | Example Run | | | | | | | | | | | |
| | | Sensitivity 01 | | | | Sensitivity 02 | | | | Sensitivity 03 | | | |
| | ADA | (Individual-01) Run 55 | | | | (Individual-02) Run 56 | | | | (Individual-03) Run 57 | | | |
| | Cone. | | % | % | | | % | % | | | % | % | |
| Titer | (ng/mL) | ECL | CV | Inhibition | Condition | ECL | CV | Inhibition | Condition | ECL | CV | Inhibition | Condition |
| 1 | 4000.00 | 74.0 | 9.6 | 99.05 | >CCP | 71.5 | 1.0 | 99.16 | >CCP | 81.0 | 5.2 | 99.05 | >CCP |
| 2 | 2000.00 | 79.0 | 9.0 | 98.86 | >CCP | 73.0 | 3.9 | 99.03 | >CCP | 85.5 | 2.5 | 98.79 | >CCP |
| 4 | 1000.00 | 74.5 | 10.4 | 98.22 | >CCP | 72.0 | 2.0 | 98.44 | >CCP | 86.0 | 0.0 | 98.06 | >CCP |
| 8 | 500.00 | 75.5 | 4.7 | 95.03 | >CCP | 72.5 | 1.0 | 95.62 | >CCP | 83.0 | 1.7 | 95.13 | >CCP |
| 16 | 250.00 | 72.0 | 5.9 | 90.30 | >CCP | 67.0 | 6.3 | 91.51 | >CCP | 83.0 | 3.4 | 89.43 | >CCP |
| 32 | 125.00 | 77.5 | 4.6 | 79.36 | >CCP | 70.5 | 5.0 | 81.32 | >CCP | 90.5 | 5.5 | 81.49 | >CCP |
| 64 | 62.50 | 76.0 | 5.6 | 62.00 | >CCP | 68.0 | 6.2 | 72.64 | >CCP | 86.5 | 0.8 | 69.43 | >CCP |
| 128 | 31.25 | 77.5 | 2.7 | 50.48 | >CCP | 71.0 | 0.0 | 56.57 | >CCP | 90.0 | 0.0 | 51.35 | >CCP |
| 256 | 15.63 | 85.5 | 10.8 | 30.20 | >CCP | 85.5 | 12.4 | 26.61 | >CCP | 90.0 | 7.9 | 31.03 | >CCP |
| 512 | 7.81 | 79.5 | 4.4 | 27.73 | >CCP | 80.5 | 2.6 | 21.08 | >CCP | 86.5 | 2.5 | 21.72 | >CCP |
| 1024 | 3.91 | 79.0 | 7.2 | 15.96 | <CCP | 72.0 | 9.8 | 18.18 | >CCP | 84.5 | 0.8 | 13.33 | <CCP |
| 2048 | 1.95 | 73.5 | 6.7 | 17.88 | >CCP | 72.5 | 4.9 | 13.69 | <CCP | 81.5 | 0.9 | 9.44 | <CCP |
| 4096 | 0.98 | 74.5 | 0.9 | 10.24 | <CCP | 73.0 | 3.9 | 7.59 | <CCP | 91.0 | 0.0 | −5.20 | <CCP |
| 8192 | 0.49 | 77.0 | 3.7 | 6.67 | <CCP | 69.5 | 3.1 | 15.76 | <CCP | 91.5 | 7.0 | −5.17 | <CCP |

The sensitivity data was also used to determine the LPC of the assay. The LPC is based on the sensitivity with a 1% false-positive rate, which was calculated in both screening and confirmatory assays using the mean of the Sensitivity+t0.99, df×SD (Sensitivity). The LPC concentration was 6.80 and 9.70 ng/ml for screening and confirmatory assay, respectively. Results are detailed in Table 31. The calculated LPC concentration is the higher LPC between the screening assay and the confirmatory assay, which is 9.70 ng/mL.

TABLE 31
LPC Determination for Screening Assay and Confirmatory Assay

| LPC Calculation | Screening Assay | Confirmatory Assay |
|---|---|---|
| Mean (Sensitivity) (ng/mL) | 2.77 | 4.25 |
| SD (Sensitivity) (ng/mL) | 1.21 | 1.61 |
| df | 5 | 5 |
| t0.99, df | 3.365 | 3.365 |
| Intermediate LPC (ng/mL) | 6.80 | 9.70 |
| Final LPC (ng/mL) | 9.70 | 9.70 |

Hook Effect Assay: The hook (prozone) effect is a phenomenon in which the signal of the assay changes from increasing to decreasing when the ADA concentration increases. The hook effect may not be critical to ADA detection if all concentrations of positive antibody yield positive results. The hook effect was assessed using a sample with an ultra-high concentration of anti-XTMAB-16 antibodies in pNHS (32,000 ng/ml), which was prepared by spiking ADA PC into pooled serum and freezing for at least 12 hours. The sample was then serially two-fold diluted to a concentration below the plate-specific TCP. All samples with a concentration of 7.81 ng/ml anti-XTMAB-16 antibodies or greater demonstrated a positive ECL value as compared to the plate-specific TCP, indicating that no prozone effect was observed. An example of data showing this result is illustrated in Table 32.

TABLE 32
Hook Effect Assay

| Run Example | | Hook Effect & Titration | | | | |
|---|---|---|---|---|---|---|
| Dilution | ADAConc. | | | Plate | Plate | |
| Factor | (ng/mL) | ECL | % CV | JC | TCP | Condition |
| 1 | 32000.00 | 11475.0 | 0.1 | 93.1 | 111.72 | >TCP |
| 2 | 16000.00 | 10057.5 | 3.1 | | | >TCP |
| 4 | 8000.00 | 9138.5 | 1.1 | | | >TCP |
| 8 | 4000.00 | 8216.5 | 2.3 | | | >TCP |
| 16 | 2000.00 | 7559.0 | 0.5 | | | >TCP |
| 32 | 1000.00 | 5498.0 | 1.2 | | | >TCP |
| 64 | 500.00 | 2717.5 | 0.2 | | | >TCP |
| 128 | 250.00 | 1290.0 | 1.3 | | | >TCP |
| 256 | 125.00 | 681.0 | 0.8 | | | >TCP |
| 512 | 62.50 | 390.5 | 1.6 | | | >TCP |
| 1024 | 31.25 | 238.5 | 4.4 | | | >TCP |
| 2048 | 15.63 | 172.0 | 0.8 | | | >TCP |
| 4096 | 7.81 | 127.5 | 2.8 | | | >TCP |
| 819 | 3.91 | 110.5 | 4.5 | | | <TCP |
| 16384 | 1.95 | 88.0 | 3.2 | | | <TCP |
| 32768 | 0.98 | 92.0 | 3.1 | | | <TCP |

Selectivity: Selectivity is the ability of the assay to detect anti-XTMAB-16 antibodies in the presence of other matrix components. ADA unspiked and spiked samples at LPC level (9.70 ng/mL) were prepared from 10 lots of normal human serum, 10 lots of disease human serum, 3 lots of hemolyzed human serum, and 3 lots of lipemic human serum that originally tested in 4 runs with both screening and confirmatory assay. For individual lots of normal human serum, 9 out of 10 unspiked samples (90%) were confirmed as negative, while 9 out of 10 spiked LPC samples (90%) were confirmed as positive. For individual lots of disease human serum, 10 out of 10 unspiked samples (100%) were confirmed as negative, while 10 out of 10 spiked LPC samples (100%) were confirmed as positive. For individual lots of hemolyzed human serum, 3 out of 3 unspiked samples (100%) were confirmed as negative, while 3 out of 3 spiked LPC samples (100%) were confirmed as positive. For individual lots of lipemic human serum, 3 out of 3 unspiked samples (100%) were confirmed as negative, while 3 out of 3 spiked LPC samples (100%) were confirmed as positive, which met the acceptance criteria. This suggests there is no matrix interference for detection of anti-XTMAB-16 antibodies in human serum. An example of data showing this result is illustrated in Table 33.

TABLE 33
Assessing Selectivity and Matrix Interference

| | | | Drug Unspiked | | | | | Drug Spiked (MAB-16 at 200 μg/mL) | | | | | Sample |
|---|---|---|---|---|---|---|---|---|---|---|---|---|---|
| Example | | | | | Plate | Plate | | | | % | CCP | | condition |
| Run | Serum ID | | ECL | % CV | NC | SCP | Condition | ECL | % CV | Inhibition | (%) | Condition | (Neg/Pos/Fail) |
| 68 | ADA | Indi 01 | 83.0 | 3.4 | 82.5 | 90.75 | <SCP | 80.0 | 5.3 | 3.61 | 16.45 | <CCP | Negative |
| | Unspiked | Indi 02 | 84.0 | 0.0 | | | <SCP | 83.0 | 6.8 | 1.19 | | <CCP | Negative |
| | | Indi 03 | 80.5 | 0.9 | | | <SCP | 84.0 | 1.7 | −4.35 | | <CCP | Negative |
| | | Indi 04 | 85.0 | 1.7 | | | <SCP | 122.0 | 34.8 | −43.53 | | Fall | Fail |
| | | Indi 05 | 78.5 | 0.9 | | | <SCP | 80.0 | 1.8 | −1.91 | | <CCP | Negative |
| | | Indi 06 | 77.5 | 6.4 | | | <SCP | 82.5 | 9.4 | −6.45 | | <CCP | Negative |
| | | Indi 07 | 75.5 | 2.8 | | | <SCP | 83.0 | 6.8 | −9.93 | | <CCP | Negative |
| | | Indi 08 | 84.0 | 8.4 | | | <SCP | 88.5 | 7.2 | −5.36 | | <CCP | Negative |
| | ADA | LPC- | 109.5 | 4.5 | | | >SCP | 89.0 | 9.5 | 18.72 | | >CCP | Positive |
| | Spiked | Indi01 | | | | | | | | | | | |
| | at LPC | LPC- | 115.5 | 5.5 | | | >SCP | 78.0 | 3.6 | 32.47 | | >CCP | Positive |
| | | Indi02 | | | | | | | | | | | |
| | | LPC- | 115.5 | 4.3 | | | >SCP | 77.0 | 9.2 | 33.33 | | >CCP | Positive |
| | | Indi03 | | | | | | | | | | | |
| | | LPC- | 117.5 | 1.8 | | | >SCP | 82.0 | 13.8 | 30.21 | | >CCP | Positive |
| | | Indi04 | | | | | | | | | | | |
| | | LPC- | 110.5 | 1.9 | | | >SCP | 78.0 | 1.8 | 29.41 | | >CCP | Positive |
| | | Indi05 | | | | | | | | | | | |
| | | LPC- | 111.0 | 8.9 | | | >SCP | 79.0 | 1.8 | 28.83 | | >CCP | Positive |
| | | Indi06 | | | | | | | | | | | |
| | | LPC- | 104.5 | 2.0 | | | >SCP | 76.5 | 6.5 | 26.79 | | >CCP | Positive |
| | | Indi07 | | | | | | | | | | | |
| | | LPC- | 113.5 | 0.6 | | | >SCP | 78.5 | 4.5 | 30.84 | | >CCP | Positive |
| | | Indi08 | | | | | | | | | | | |
| 69 | ADA | Indi 09 | 93.5 | 2.3 | 89.4 | 98.34 | <SCP | 81.5 | 6.1 | 12.83 | | <CCP | Negative |
| | Unspiked | Indi 10 | 87.0 | 1.6 | | | <SCP | 85.5 | 2.5 | 1.72 | | <CCP | Negative |
| | | Disease-01 | 90.5 | 7.0 | | | <SCP | 81.5 | 0.9 | 9.94 | | <CCP | Negative |
| | | Disease-02 | 96.5 | 6.6 | | | <SCP | 84.5 | 2.5 | 12.44 | | <CCP | Negative |
| | | Disease-03 | 92.0 | 12.3 | | | <SCP | 83.5 | 11.0 | 9.24 | | <CCP | Negative |
| | | Disease-04 | 91.0 | 0.0 | | | <SCP | 87.0 | 1.6 | 4.40 | | <CCP | Negative |
| | | Disease-05 | 80.0 | 1.8 | | | <SCP | 86.0 | 1.6 | −7.50 | | <CCP | Negative |
| | | Disease-06 | 85.5 | 5.8 | | | <SCP | 92.5 | 3.8 | −8.19 | | <CCP | Negative |
| | ADA | LPC- | 120.0 | 7.1 | | | >SCP | 97.0 | 24.8 | 19.17 | | Fail | Fail |
| | Spiked | Indi09 | | | | | | | | | | | |
| | at LPC | LPC- | 121.0 | 0.0 | | | >SCP | 81.0 | 8.7 | 33.06 | | >CCP | Positive |
| | | Indi10 | | | | | | | | | | | |
| | | LPC- | 116.0 | 0.0 | | | >SCP | 78.5 | 4.5 | 32.33 | | >CCP | Positive |
| | | Disease-01 | | | | | | | | | | | |
| | | LPC- | 121.0 | 2.3 | | | >SCP | 80.0 | 1.8 | 33.88 | | >CCP | Positive |
| | | Disease-02 | | | | | | | | | | | |
| | | LPC- | 113.5 | 3.1 | | | >SCP | 84.0 | 3.4 | 25.99 | | >CCP | Positive |
| | | Disease-03 | | | | | | | | | | | |
| | | LPC- | 118.5 | 0.6 | | | >SCP | 86.5 | 7.4 | 27.00 | | >CCP | Positive |
| | | Disease-04 | | | | | | | | | | | |
| | | LPG- | 109.5 | 0.6 | | | >SCP | 81.0 | 3.5 | 26.03 | | >CCP | Positive |
| | | Disease-05 | | | | | | | | | | | |
| | | LPC- | 112.5 | 0.6 | | | >SCP | 86.0 | 0.0 | 23.56 | | >CCP | Positive |
| | | Disease-06 | | | | | | | | | | | |

Drug Tolerance: The presence of drug XTMAB-16 in samples may interfere with the detection of anti-XTMAB-16 antibodies and, hence, the ability of the assay system to correctly detect the ADA was evaluated. Drug tolerance limit was defined as the highest level of drug in the presence of which ADA is still detected. Potential interference by the drug was evaluated using a positive control prepared at concentrations of 100 ng/ml and 9.7 ng/mL. These controls were spiked with XTMAB-16 at concentrations ranging from 5.0 μg/mL to 400 μg/mL. The addition of up to 100.0 μg/mL XTMAB-16 had no impact on the ADA detection in samples spiked at 100 ng/ml, and the addition of up to 5.0 μg/mL XTMAB-16 had impact on the ADA detection in samples spiked at 9.7 ng/ml, which led to a false-negative result. An example of data showing this result is illustrated in Table 34.

TABLE 34
Assessing Drug Tolerance

| | Example Run | | | | | | | |
|---|---|---|---|---|---|---|---|---|
| | Drug Tolerance | | | | | | | |
| Concentration | ADA PC at 100 ng/mL | | | ADA PC at 9.70 ng/mL | | | Plate | Plate |
| (μg/mL) | ECL | % CV | Condition | ECL | % CV | Condition | NC | SCP |
| 400.0 | 85.5 | 5.8 | <SCP | 91.0 | 4.7 | <SCP | 91.1 | 100.21 |
| 200.0 | 98.5 | 0.7 | <SCP | 88.0 | 1.6 | <SCP | | |
| 100.0 | 104.0 | 5.4 | >SCP | 94.5 | 14.2 | <SCP | | |
| 50.0 | 109.5 | 1.9 | >SCP | 92.0 | 4.6 | <SCP | | |
| 20.0 | 112.0 | 0.0 | >SCP | 95.0 | 6.0 | <SCP | | |
| 10.0 | 109.0 | 1.3 | >SCP | 95.5 | 5.2 | <SCP | | |
| 5.0 | 103.0 | 4.1 | >SCP | 99.0 | 8.6 | <SCP | | |
| 0.0 | 323.5 | 2.8 | >SCP | 127.5 | 2.8 | >SCP | | |

Intra-assay precision: Intra-assay precision, an evaluation of the within-run performance of the controls, was evaluated in one run using six independent preparations of the HPC (4,000 ng/mL), LPC (9.70 ng/mL), and NC. The results demonstrate that the controls met the intra-assay precision criteria of ≤20.0% for the ECL signal for both screening and confirmatory assays. The controls also met the criteria of ECL of NC<plate-specific SCP≤LPC<HPC for screening assay, and % inhibition of NC<CCP≤LPC<and HPC for confirmatory assay. The screening intra-assay precision of the HPC and LPC was 18.2% and 4.3%, respectively. The confirmatory intra-assay precision of the ECL signal for HPC and LPC was 6.3% and 4.1%, respectively. The intra-assay precision of percent for HPC and LPC was 0.2% and 9.1%, respectively. An example of data showing these results is illustrated in Table 35.

TABLE 35
Assessing Intra-assay Precision

| | Example Run | | | | | | | | | | | | | | |
|---|---|---|---|---|---|---|---|---|---|---|---|---|---|---|---|
| | Drug Unspiked | | | | | | Drug Spiked (MAB-16 at 200 μg/mL) | | | | | | | | |
| | HPC | | LPC | | NC | | HPC | | | LPC | | | NC | | |
| | ECL | % | ECL | % | ECL | % | ECL | % | % | ECL | % | % | ECL | % | % |
| Replicate | mean | CV | mean | CV | mean | CV | mean | CV | Inhibition | mean | CV | Inhibition | mean | CV | Inhibition |
| 1 | 9496.5 | 11.8 | 144.5 | 0.5 | 98.5 | 0.7 | 90.0 | 6.3 | 99.05 | 89.5 | 2.4 | 38.06 | 89.0 | 6.4 | 9.64 |
| 2 | 12278.0 | 34.5 | 140.0 | 3.0 | 98.0 | 10.1 | 88.0 | 3.2 | 99.28 | 88.5 | 2.4 | 36.79 | 92.0 | 4.6 | 6.12 |
| 3 | 8407.5 | 1.7 | 137.5 | 3.6 | 92.5 | 2.3 | 100.5 | 3.5 | 98.80 | 90.5 | 2.3 | 34.18 | 102.0 | 9.7 | −10.27 |
| 4 | 8686.0 | 0.0 | 136.0 | 0.5 | 94.5 | 6.0 | 85.0 | 0.8 | 99.02 | 80.5 | 5.3 | 40.81 | 82.0 | 0.9 | 13.23 |
| 5 | 7914.0 | 0.1 | 132.0 | 3.2 | 89.0 | 0.8 | 90.5 | 0.8 | 98.86 | 88.5 | 1.6 | 32.95 | 91.0 | 0.0 | −2.25 |
| 6 | 7880.5 | 0.1 | 128.0 | 0.6 | 91.5 | 3.9 | 91.5 | 5.4 | 98.84 | 86.5 | 8.2 | 32.42 | 106.5 | 4.6 | −16.39 |
| Mean | 9110.4 | NA | 136.3 | NA | 94.0 | NA | 90.9 | NA | 98.98 | 87.3 | NA | 35.87 | 93.8 | NA | 0.0 |
| % CV | 18.2 | NA | 4.3 | NA | NA | NA | 5.7 | NA | 0.2 | 4.1 | NA | 9.1 | NA | NA | NA |

Inter-assay precision: Inter-assay precision, an evaluation of the between-run performance of the controls, was evaluated based on the HPC (4,000 ng/ml), LPC (9.7 ng/ml), and NC results across all accepted validation runs, excluding PCs from the robustness run. The screening inter-assay precision of the method was 14.1% (HPC) and 7.9% (LPC). The controls met the inter-assay precision criteria of ≤20.0% for HPC and LPC. The confirmatory inter-assay precision of the % inhibition for HPC and LPC was 0.2% and 13.3%, respectively. The controls also met the criteria of ECL NC<plate-specific SCP≤LPC<HPC for screening assay, and % inhibition of NC<CCP≤LPC and HPC for confirmatory assay, respectively. An example of data showing this result is illustrated in Table 36.

TABLE 36
Assessing Inter-assay Precision

| Example | | Screening Assay | | | | | | | |
|---|---|---|---|---|---|---|---|---|---|
| Run | Run Parameter | HPC | HPC | LPC | LPC | NC | NC | NC | NC |
| 34 | CP 1 | 8561.0 | 7558.0 | NA | NA | 97.5 | 90.5 | 83.0 | 86.5 |
| 35 | CP 2 | 7783.5 | 7819.0 | NA | NA | 82.5 | 80.0 | 76.5 | 82.0 |
| 36 | CP 3 | 8032.5 | 8030.0 | NA | NA | 81.5 | 78.0 | 75.5 | 76.0 |
| 37 | CP 4 | 8252.5 | 8187.0 | NA | NA | 87.0 | 81.0 | 80.5 | 84.0 |
| 38 | CP 5 | 8714.0 | 8668.5 | NA | NA | 80.0 | 79.0 | 78.0 | 82.0 |
| 39 | CP 6 | 8866.5 | 8049.0 | NA | NA | 77.5 | 71.0 | 77.5 | 76.5 |
| 40 | CP 7 | 9051.0 | 8226.0 | NA | NA | 89.5 | 86.0 | 78.0 | 78.5 |
| 41 | CP 8 | 8736.0 | 8364.0 | NA | NA | 93.0 | 88.0 | 91.5 | 91.5 |
| 42 | CP 9 | 8833.0 | 8025.5 | NA | NA | 91.0 | 84.0 | 84.0 | 84.5 |
| 43 | CP 10 | 10524.5 | 9894.0 | NA | NA | 85.0 | 83.5 | 81.5 | 81.0 |
| 44 | CP 11 | 10455.0 | 10403.5 | NA | NA | 89.0 | 86.5 | 85.5 | 83.5 |
| 45 | CP 12 | 10864.0 | 10656.5 | NA | NA | 90.0 | 88.5 | 91.0 | 91.0 |
| 49 | CP 13 | 8053.0 | 7539.0 | NA | NA | 87.0 | 85.5 | 88.0 | 86.5 |
| 50 | CP 14 | 9018.5 | 8162.5 | NA | NA | 102.5 | 93.5 | 85.5 | 89.0 |
| 51 | CP 15 | 8498.0 | 7558.5 | NA | NA | 88.5 | 83.5 | 78.5 | 82.0 |
| 52 | CP 16 | 8083.0 | 7484.0 | NA | NA | 85.0 | 84.0 | 79.5 | 80.5 |
| 53 | CP 17 | 8310.5 | 7673.5 | NA | NA | 85.5 | 82.0 | 80.0 | 88.0 |
| 54 | CP 18 | 8127.5 | 7328.5 | NA | NA | 82.0 | 79.0 | 77.5 | 80.5 |
| 55 | Individual Sensitivity- 1 | 8187.5 | 7516.0 | NA | NA | 90.5 | 84.5 | 76.0 | 80.0 |
| 56 | Individual Sensitivity-2 | 8348.5 | 8288.5 | NA | NA | 84.5 | 79.5 | 81.0 | 82.0 |
| 57 | Individual Sensitivity-3 | 7817.5 | 6080.0 | NA | NA | 88.5 | 85.5 | 88.0 | 93.5 |
| 58 | Pooled Sensitivity-1 | 8279.0 | 7531.0 | NA | NA | 89.0 | 92.5 | 97.0 | 93.0 |
| 59 | Pooled Sensitivity-2 | 8314.5 | 7464.0 | NA | NA | 99.0 | 93.0 | 82.5 | 85.5 |
| 60 | Pooled Sensitivity-3 | 8642.5 | 7899.0 | NA | NA | 103.5 | 102.5 | 93.0 | 93.0 |

Stability: The stability of positive controls in human serum was used to assess sample stability. Freeze/thaw (6 cycles of −70° C.±10° C. to ambient temperature) and bench-top (24 hours at ambient temperature) stability were tested. The acceptance criteria for the stability samples were as follows: (1) NC<plate-specific SCP≤LPC<HPC for screening assay, (2) % inhibition of NC<CCP≤LPC and HPC for confirmatory assay, (3) % CV≤20% for the NC, LPC, and HPC, (4) at least 66.7% (two out of three) stability samples must meet the above acceptance criteria.

The tested samples were stored at −70° C. (nominal) for at least 24 hours prior to first thaw and 12 hours prior to each additional thaw. Samples were at ambient temperature for 1-2 hours prior to refreezing. Three aliquots for each control (HPC, LPC, and NC) were tested after undergoing six freeze/thaw cycles on a plate using both screening and confirmatory assay, with frozen PCs and NC, which were used for plate acceptance. The results met the acceptance criteria, therefore, anti-XTMAB-16 antibodies in human serum are stable for up to six freeze/thaw cycles from −70° C. (nominal) to ambient temperature. An example of these results is shown in Table 37.

TABLE 37
Assessing Stability (Example A)

| Freeze/Thaw (6-Cycles) | | Drug Unspiked | | | | | Drug Spiked (MAB-16 at 200 μg/mL) | | | | |
|---|---|---|---|---|---|---|---|---|---|---|---|
| Example | Sample | ECL | % | Plate | Plate | | ECL | % | % | Confirmatory | |
| Run | ID | mean | CV | NC | SCP | Condition | mean | CV | Inhibition | CP (%) | Condition |
| 67 | HPC-1 | 8775.0 | 0.9 | 92.9 | 102.19 | Pass | 91.5 | 10.0 | 98.96 | 16.45 | Pass |
| | HPC-2 | 8360.5 | 0.2 | | | Pass | 94.5 | 0.7 | 98.87 | | Pass |
| | HPC-3 | 9073.0 | 3.9 | | | Pass | 106.0 | 20.0 | 98.83 | | Pass |
| | LPC-1 | 133.5 | 2.6 | | | Pass | 90.0 | 3.1 | 32.58 | | Pass |
| | LPC-2 | 128.5 | 0.6 | | | Pass | 88.5 | 0.8 | 31.13 | | Pass |
| | LPC-3 | 133.0 | 2.1 | | | Pass | 95.0 | 3.0 | 28.57 | | Pass |
| | NC-1 | 93.0 | 4.6 | | | Pass | 90.0 | 3.1 | 3.23 | | Pass |
| | NC-2 | 94.5 | 0.7 | | | Pass | 95.0 | 6.0 | −0.53 | | Pass |
| | NC-3 | 87.5 | 0.8 | | | Pass | 91.0 | 4.7 | −4.00 | | Pass |

The tested samples were stored at −70° C. (nominal) for at least 24 hours prior to first thaw. Three aliquots of each pre-existing frozen control (HPC, LPC, and NC) were thawed on the bench-top for 24.5 hours, prior to analysis on a plate using both screening and confirmatory assay with frozen PCs and NC, which were used for plate acceptance. All HPC, LPC, and NC stability samples met the acceptance criteria, therefore, anti-XTMAB-16 antibodies in human serum are stable for up to 24.5 hours at ambient surroundings. An example of these results is shown in Table 38.

TABLE 38
Assessing Stability (Example B)

| Bench top (24.5 hours) | | Drug Unspiked | | | | | Drug Spiked (MAB-16 at 200 μg/mL) | | | | |
|---|---|---|---|---|---|---|---|---|---|---|---|
| Example | Sample | ECL | % | Plate | Plate | | ECL | % | % | Confirmatory | |
| Run | ID | mean | CV | NC | SCP | Condition | mean | CV | Inhibition | CP (%) | Condition |
| 66 | HPC-1 | 8074.5 | 0.5 | 90.1 | 99.11 | Pass | 84.5 | 5.9 | 98.95 | 16.45 | Pass |
| | HPC-2 | 8175.5 | 2.8 | | | Pass | 85.0 | 5.0 | 98.96 | | Pass |
| | HPC-3 | 8311.5 | 4.8 | | | Pass | 114.0 | 29.8 | 98.63 | | Fail |
| | LPC-1 | 119.0 | 1.2 | | | Pass | 83.5 | 4.2 | 29.83 | | Pass |
| | LPC-2 | 121.0 | 2.3 | | | Pass | 76.0 | 7.4 | 37.19 | | Pass |
| | LPC-3 | 122.0 | 2.3 | | | Pass | 92.5 | 3.8 | 24.18 | | Pass |
| | NC-1 | 89.0 | 6.4 | | | Pass | 84.5 | 4.2 | 5.06 | | Pass |
| | NC-2 | 93.5 | 2.3 | | | Pass | 83.0 | 3.4 | 11.23 | | Pass |
| | NC-3 | 86.5 | 5.7 | | | Pass | 88.0 | 0.0 | −1.73 | | Pass |

Study Phase Bioanalysis Acceptance Criteria for Controls: System suitability criteria was established using data from all valid runs in the validation except robustness runs. In future sample analysis, the criteria are utilized as acceptance criteria for the acceptance or rejection of a run. The % CV criterion for intra-replicate measurements of controls and samples is ≤20.0%.

Acceptance criteria for NC included an in screening and titration assay and an ECL of NC<plate-specific SCP, and an ECL of NC mean≤142.2. These results are further outlined in Table 39.

TABLE 39
Plate Control Acceptance Ranges

| System Suitability | | | |
|---|---|---|---|
| Criteria | HPC | LPC | NC |
| Mean | 8118.6 | 126.8 | 86.4 |
| SD | 1141.0 | 10.0 | 18.6 |
| Upper Limit | NA | 156.8 | 142.2 |
| (Mean + 3 × SD) | | | |
| Lower Limit | 4695.6 | 96.8 | NA |
| (Mean − 3 × SD) | | | |

Acceptance criteria for NC also included a confirmatory assay with % inhibition of NC<CCP (16.45%).

Acceptance criteria for LPC included an in screening and titration assay and an ECL of LPC≥plate-specific SCP and an LPC where the ECL mean≥96.8 or ≤156.8.

Acceptance criteria for LPC also included an in confirmatory assay, and % inhibition of LPC≥CCP (16.45%).

Acceptance criteria for HPC included an in screening and titration assay with an ECL of HPC>ECL of LPC, and HPC of 4695.6≤ECL mean.

Acceptance criteria for HPC also included an in confirmatory assay with % inhibition of HPC≥CCP (16.45%).

Acceptance criteria for titer control included the titer control value equaling±2×overall mean titers from sensitivity runs (inclusive), and the titer of XTMAB-16 ADA PC at 4000 ng/mL in pNHS is 25600-102400.

Assay Robustness: To determine assay robustness, the parameters of the assay were tested. The HPC, LPC, and NC were tested in both screening and confirmatory assay with different incubation times in two runs. The results of both runs met the method acceptance criteria, and the controls were within the established ranges, indicating that the assay demonstrated robustness. Examples of these results are shown in Tables 40 and 41.

TABLE 40
Assessing Assay Robustness (Example A)

| | | Drug Unspiked | | | | | Drug Spiked (MAB-16 at 200 μg/mL) | | | | | |
|---|---|---|---|---|---|---|---|---|---|---|---|---|
| Example | Sample | | % | Plate | Plate | | | % | % | CCP | | Run |
| Run | ID | ECL | CV | NC | SCP | Condition | ECL | CV | Inhibition | (%) | Condition | Condition |
| 61 | HPC | 8253.0 | 1.6 | 111.8 | 123.02 | Pass | 101.0 | 1.4 | 98.78 | 16.45 | Pass | Pass |
| | HPC | 8929.0 | 7.5 | | | Pass | 107.5 | 9.9 | 98.80 | | Pass | |
| | HPC | 7354.5 | 1.6 | | | Pass | 94.5 | 0.7 | 98.72 | | Pass | |
| | LPC | 132.5 | 10.1 | | | Pass | 106.0 | 4.0 | 20.00 | | Pass | |
| | LPC | 125.0 | 5.7 | | | Pass | 102.5 | 2.1 | 18.00 | | Pass | |
| | LPC | 120.0 | 5.9 | | | Fail | 90.5 | 0.8 | 24.58 | | Pass | |
| | NC | 100.0 | 0.0 | | | Pass | 105.5 | 6.0 | −5.50 | | Pass | |
| | NC | 111.5 | 20.9 | | | Fail | 100.0 | 2.8 | 10.31 | | Fail | |
| | NC | 113.5 | 15.6 | | | Pass | 97.5 | 0.7 | 14.10 | | Pass | |
| | NC | 114.0 | 1.2 | | | Pass | 124.0 | 26.2 | −8.77 | | Fail | |
| | NC | 121.0 | 10.5 | | | Pass | 115.0 | 1.2 | 4.96 | | Pass | |
| | NC | 111.0 | 2.5 | | | Pass | 128.0 | 3.3 | −15.32 | | Pass | |
| 62 | HPC | 7926.0 | 0.7 | 95.1 | 104.59 | Pass | 87.0 | 1.6 | 98.90 | | Pass | Pass |
| | HPC | 7689.5 | 1.0 | | | Pass | 94.0 | 6.0 | 98.78 | | Pass | |
| | HPC | 6926.0 | 3.2 | | | Pass | 85.5 | 0.8 | 98.77 | | Pass | |
| | LPC | 109.5 | 0.6 | | | Pass | 88.0 | 3.2 | 19.63 | | Pass | |
| | LPC | 116.5 | 3.0 | | | Pass | 86.5 | 5.7 | 25.75 | | Pass | |
| | LPC | 103.5 | 3.4 | | | Fail | 81.5 | 6.1 | 21.26 | | Pass | |
| | NC | 113.0 | 32.5 | | | Fail | 85.5 | 0.8 | 24.34 | | Fail | |
| | NC | 91.0 | 4.7 | | | Pass | 86.0 | 0.0 | 5.49 | | Pass | |
| | NC | 88.0 | 3.2 | | | Pass | 90.5 | 2.3 | −2.84 | | Pass | |
| | NC | 94.5 | 0.7 | | | Pass | 91.5 | 0.8 | 3.17 | | Pass | |
| | NC | 89.0 | 1.6 | | | Pass | 96.5 | 3.7 | −8.43 | | Pass | |
| | NC | 95.0 | 3.0 | | | Pass | 101.5 | 7.7 | −6.84 | | Pass | |

TABLE 41
Assessing Assay Robustness (Example B)

| | Min Incubation | Max Incubation |
|---|---|---|
| | Time | Time |
| Incubation Step | (Run 61) | (Run 62) |
| Controls/samples incubate with glycine buffer | 28 min | 65 min |
| MSD plate incubate with blocking buffer | 56 min | 172 min |
| Controls/samples incubate with Bio-MAB-16 | 4 min | 17 min |
| solution in polypropylene plate | | |
| Controls/samples incubate in MSD plate | 54 min | 128 min |
| Controls/samples incubate with Ru-MAB-16 | 49 min | 134 min |
| solution | | |

### ADDITIONAL EMBODIMENTS

The present technology includes, but is not limited to, the following specific embodiments set forth herein below in paragraphs [0334]-[0410]:

1. A method for detecting an anti-drug antibody (ADA) that binds a target antibody comprising an anti-TNFα antibody in a sample comprising:
(i) incubating the sample with a composition comprising:
   (a) a first affinity antibody comprising an anti-TNFα antibody conjugated to a first affinity label, and
   (b) a detection antibody comprising an anti-TNFα antibody conjugated to a detection label;
(ii) isolating ADAs that form a complex with both the first affinity antibody and the detection antibody, wherein the ADA is isolated by contacting the complex with a second affinity label that binds to the first affinity label; and
(iii) detecting the ADA by measuring a level of the detection label above a predetermined cut-point.

2. The method of embodiment 1, wherein isolating the ADAs comprises:
(a) providing a solid phase comprising the second affinity label;
(b) contacting the sample and the composition with the solid phase; and
(c) washing the solid phase.

3. The method of embodiment 1 or 2, wherein the target antibody, the affinity antibody, and/or the detection antibody comprise a chimeric human-murine monoclonal IgG1-kappa antibody.

4. The method of any one of embodiments 1-3, wherein the target antibody, the affinity antibody, and/or the detection antibody comprise SEQ ID NOs: 1 and 4.

5. The method of any one of embodiments 2-4, wherein the solid phase is a plate.

6. The method of any one of embodiments 1-5, wherein the first affinity label is biotin.

7. The method of any one of embodiments 1-6, wherein the second affinity label is streptavidin.

8. The method of any one of embodiments 1-7, wherein the detection antibody comprises a sulfo-Tag labeled anti-TNFα antibody.

9. The method of any one of embodiments 2-8, wherein measuring the level of the detection label comprises:
(1) contacting the solid phase with a marker configured to produce a chemiluminescent or electrochemiluminescent signal when the marker is proximal to the detection label; and
(2) detecting the chemiluminescent or electrochemiluminescent signal.

10. The method of embodiment 9, wherein the marker is tripropylamine (TPA), and measuring the level of the detection label further comprises applying an electrical current to the marker to produce the chemiluminescent or electrochemiluminescent signal.

11. The method of any one of embodiments 2-10, wherein the solid phase is blocked with a blocking buffer before the sample and the composition are contacted with the solid phase.

12. The method of any one of embodiments 1-11, further comprising performing steps (i) to (iii) on a control sample.

13. The method of embodiment 12, wherein the control sample is incubated with the composition and contacted with the second affinity label.

14. The method of any one of embodiments 1-13, wherein the predetermined cut-point corresponds to a level with a 5% false-positive rate.

15. The method of embodiment 14, wherein the 5% false-positive rate is determined by measuring the detection label in a plurality of control samples and calculating the 5% false-positive rate.

16. The method of embodiment 9, wherein the chemiluminescent or electrochemiluminescent signal is measured as electrochemiluminescence (ECL).

17. The method of embodiment 16, wherein the predetermined cut-point is an ECL value of at least 1.

18. The method of embodiment 16 or 17, wherein the predetermined cut-point is an ECL value of about 1.10.

19. The method of any one of embodiments 1-18, wherein the method has a sensitivity that is less than 6 ng/mL.

20. The method of embodiment 19, wherein the sensitivity is less than 4 ng/ml.

21. The method of any one of embodiments 1-20, wherein the method has a drug tolerance of about 100 ng/mL.

22. A method for detecting an anti-drug antibody (ADA) that binds a target antibody comprising an anti-TNFα antibody in a sample comprising:
(i) contacting a sample with one or more anti-TNFα conjugates to form a complex between the ADA and the anti-TNFα conjugates; and
(ii) measuring a level of anti-TNFα conjugates in the complex;
wherein the method detects the ADA with a sensitivity less than 5 ng/ml.

23. The method of embodiment 22, wherein the one or more anti-TNFα conjugates comprise an affinity antibody comprising an anti-TNFα antibody conjugated to an affinity label.

24. The method of embodiment 22 or 23, wherein the one or more anti-TNFα conjugates comprise a detection antibody comprising an anti-TNFα antibody conjugated to a detection label.

25. The method of embodiment 23, wherein the affinity label comprises biotin.

26. The method of embodiment 24, wherein the detection label comprises a sulfo-Tag.

27. The method of any one of embodiments 22-26, wherein the method detects the ADA with a sensitivity less than 3 ng/mL.

28. The method of any one of embodiments 22-27, wherein the method detects the ADA with a sensitivity of 2.77 ng/ml or less.

29. A method for estimating a quantity of an anti-drug antibody (ADA) that binds a target antibody comprising an anti-TNFα antibody in a sample comprising:
(i) incubating the sample with a composition comprising:
   (a) an affinity antibody comprising an anti-TNFα antibody conjugated to a first affinity label, and
   (b) a detection antibody comprising an anti-TNFα antibody conjugated to a detection label;
(ii) isolating ADAs that form a complex with both the affinity antibody and the detection antibody, wherein the ADA is isolated by contacting the complex with a second affinity label that binds to the first affinity label;
(iii) detecting the ADA by measuring a level of the detection label above a predetermined cut-point;
(iv) performing steps (i)-(iii) on a known sample having a known concentration of ADA; and
(v) comparing the level of the detection label in the sample to the level of the detection label in the known sample.

30. The method of embodiment 29, wherein the known sample is serially diluted into a plurality of titer samples, wherein comparing the level of the detection label in the sample to the level of the detection label in the known sample comprises generating a curve of the levels of the detection label in each of the titer samples and comparing the detection label in the sample to the curve.

31. A method of confirming that an anti-drug antibody (ADA) binds a target antibody comprising an anti-TNFα antibody in a sample by performing a competitive binding assay comprising performing the method of embodiment 1 on the sample, wherein the composition further comprises an unlabeled anti-TNFα antibody.

32. The method of embodiment 31, wherein the ADA is confirmed in the sample by measuring a level of the detection label that is lower by a predetermined factor than the level of the detection label when the unlabeled anti-TNFα antibody is not present.

33. A kit for detecting an anti-drug antibody (ADA) that binds a target antibody comprising an anti-TNFα antibody in a sample comprising:
(i) an affinity antibody comprising an anti-TNFα antibody conjugated to a first affinity label;
(ii) a detection antibody comprising an anti-TNFα antibody conjugated to a detection label; and
(iii) a solid phase comprising a second affinity label, wherein the second affinity label binds to the first affinity label.

34. The kit of embodiment 33, wherein the solid phase is a plate.

35. The kit of embodiment 33 or 34, wherein the second affinity label is streptavidin.

36. The kit of any one of embodiments 33-35, wherein the first affinity label is biotin.

37. The kit of any one of embodiments 33-36, wherein the detection antibody comprises a sulfo-Tag labeled anti-TNFα antibody.

38. The kit of any one of embodiments 33-37, further comprising a marker configured to produce a chemiluminescent or electrochemiluminescent signal where the marker is proximal to the detection label.

39. The kit of embodiment 38, wherein the marker is tripropylamine (TPA).

40. The kit of any one of embodiments 33-39, further comprising a blocking buffer.

41. The kit of any one of embodiments 33-40, further comprising a wash buffer.

42. A kit for estimating a quantity of an anti-drug antibody (ADA) that binds a target antibody comprising an anti-TNFα antibody in a sample comprising:
(i) an affinity antibody comprising an anti-TNFα antibody conjugated to a first affinity label;
(ii) a detection antibody comprising an anti-TNFα antibody conjugated to a detection label;
(iii) a solid phase comprising a second affinity label, wherein the second affinity label binds to the first affinity label; and
(iv) a positive control.

43. The kit of embodiment 42, wherein the positive control is a plurality of titer samples.

44. A kit for confirming that an anti-drug antibody (ADA) binds a target antibody comprising an anti-TNFα antibody comprising:
(i) an affinity antibody comprising an anti-TNFα antibody conjugated to a first affinity label;
(ii) a detection antibody comprising an anti-TNFα antibody conjugated to a detection label;
(iii) an unlabeled anti-TNFα antibody; and
(iv) a solid phase comprising a second affinity label, wherein the second affinity label binds to the first affinity label.

45. Use of a target antibody comprising an anti-TNFα antibody for detecting an ADA that binds the target antibody in a sample comprising:
(i) incubating the sample with a composition comprising:
   (a) a first affinity antibody comprising an anti-TNFα antibody conjugated to a first affinity label, and
   (b) a detection antibody comprising an anti-TNFα antibody conjugated to a detection label;
(ii) isolating ADAs that form a complex with both the first affinity antibody and the detection antibody, wherein the ADA is isolated by contacting the complex with a second affinity label that binds to the first affinity label; and
(iii) detecting the ADA by measuring a level of the detection label above a predetermined cut-point.

46. The use of embodiment 45, wherein isolating the ADAs comprises:
(a) providing a solid phase comprising the second affinity label;
(b) contacting the sample and the composition with the solid phase; and
(c) washing the solid phase.

47. The use of embodiment 45 or 46, wherein the target antibody, the affinity antibody, and/or the detection antibody comprise a chimeric human-murine monoclonal IgG1-kappa antibody.

48. The use of any one of embodiments 45-47, wherein the target antibody, the affinity antibody, and/or the detection antibody comprise SEQ ID NOs: 1 and 4.

49. The use of any one of embodiments 46-48, wherein the solid phase is a plate.

50. The use of any one of embodiments 45-49, wherein the first affinity label is biotin.

51. The use of any one of embodiments 45-50, wherein the second affinity label is streptavidin.

52. The use of any one of embodiments 45-51, wherein the detection antibody comprises a sulfo-Tag labeled anti-TNFα antibody.

53. The use of any one of embodiments 46-52, wherein measuring the level of the detection label comprises:
(1) contacting the solid phase with a marker configured to produce a chemiluminescent or electrochemiluminescent signal when the marker is proximal to the detection label; and (2) detecting the chemiluminescent or electrochemiluminescent signal.

54. The use of embodiment 53, wherein the marker is tripropylamine (TPA), and measuring the level of the detection label further comprises applying an electrical current to the marker to produce the chemiluminescent or electrochemiluminescent signal.

55. The use of any one of embodiments 46-54, wherein the solid phase is blocked with a blocking buffer before the sample and the composition are contacted with the solid phase.

56. The use of any one of embodiments 45-55, further comprising performing steps (i) to (iii) on a control sample.

57. The use of embodiment 56, wherein the control sample is incubated with the composition and contacted with the second affinity label.

58. The use of any one of embodiments 45-57, wherein the predetermined cut-point corresponds to a level with a 5% false-positive rate.

59. The use of embodiment 58, wherein the 5% false-positive rate is determined by measuring the detection label in a plurality of control samples and calculating the 5% false-positive rate.

60. The use of embodiment 53, wherein the chemiluminescent or electrochemiluminescent signal is measured as electrochemiluminescence (ECL).

61. The use of embodiment 60, wherein the predetermined cut-point is an ECL value of at least 1.

62. The use of embodiment 60 or 61, wherein the predetermined cut-point is an ECL value of about 1.10.

63. The use of any one of embodiments 45-62, wherein the method has a sensitivity that is less than 6 ng/mL.

64. The use of embodiment 63, wherein the sensitivity is less than 4 ng/mL.

65. The use of any one of embodiments 45-64, wherein the method has a drug tolerance of about 100 ng/mL.

66. Use of one or more anti-TNFα conjugates for detecting an anti-drug antibody (ADA) that binds a target antibody comprising an anti-TNFα antibody in a sample comprising:
(i) contacting a sample with the one or more anti-TNFα conjugates to form a complex between the ADA and the anti-TNFα conjugates; and
(ii) measuring a level of anti-TNFα conjugates in the complex;
wherein the method detects the ADA with a sensitivity less than 5 ng/mL.

67. The use of embodiment 66, wherein the one or more anti-TNFα conjugates comprise an affinity antibody comprising an anti-TNFα antibody conjugated to an affinity label.

68. The use of embodiment 66 or 67, wherein the one or more anti-TNFα conjugates comprise a detection antibody comprising an anti-TNFα antibody conjugated to a detection label.

69. The use of embodiment 67, wherein the affinity label comprises biotin.

70. The use of embodiment 68, wherein the detection label comprises a sulfo-Tag.

71. The use of any one of embodiments 66-70, wherein the method detects the ADA with a sensitivity less than 3 ng/mL.

72. The use of any one of embodiments 66-71, wherein the method detects the ADA with a sensitivity of 2.77 ng/ml or less.

73. Use of a target antibody comprising an anti-TNFα antibody for estimating a quantity of an anti-drug antibody (ADA) that binds the target antibody comprising the anti-TNFα antibody in a sample comprising:
(i) incubating the sample with a composition comprising:
   (a) an affinity antibody comprising an anti-TNFα antibody conjugated to a first affinity label, and
   (b) a detection antibody comprising an anti-TNFα antibody conjugated to a detection label;
(ii) isolating ADAs that form a complex with both the affinity antibody and the detection antibody, wherein the ADA is isolated by contacting the complex with a second affinity label that binds to the first affinity label;
(iii) detecting the ADA by measuring a level of the detection label above a predetermined cut-point;
(iv) performing steps (i)-(iii) on a known sample having a known concentration of ADA; and
(v) comparing the level of the detection label in the sample to the level of the detection label in the known sample.

74. The use of embodiment 73, wherein the known sample is serially diluted into a plurality of titer samples, wherein comparing the level of the detection label in the sample to the level of the detection label in the known sample comprises generating a curve of the levels of the detection label in each of the titer samples and comparing the detection label in the sample to the curve.

75. Use of a target antibody comprising an anti-TNFα antibody for confirming that an anti-drug antibody (ADA) binds the target antibody comprising the anti-TNFα antibody in a sample by performing a competitive binding assay comprising performing the method of embodiment 1 on the sample, wherein the composition further comprises an unlabeled anti-TNFα antibody.

76. The use of embodiment 75, wherein the ADA is confirmed in the sample by measuring a level of the detection label that is lower by a predetermined factor than the level of the detection label when the unlabeled anti-TNFα antibody is not present.

From the foregoing, it will be appreciated that specific embodiments of the present technology have been described herein for purposes of illustration, but that various modifications may be made without deviating from the scope of the present technology. Accordingly, the present technology is not limited except as by the appended claims.

What is claimed is:

1. A method for detecting an anti-drug antibody (ADA) that binds a target antibody comprising an anti-TNFα antibody in a sample comprising:
(i) incubating the sample with a composition comprising:
(a) a first affinity antibody comprising an anti-TNFα antibody conjugated to a first affinity label, and
(b) a detection antibody comprising an anti-TNFα antibody conjugated to a detection label;
(ii) isolating ADAs that form a complex with both the first affinity antibody and the detection antibody, wherein the ADA is isolated by contacting the complex with a second affinity label that binds to the first affinity label; and
(iii) detecting the ADA by measuring a level of the detection label above a predetermined cut-point.

2. The method of claim 1, wherein isolating the ADAs comprises:
(a) providing a solid phase comprising the second affinity label;
(b) contacting the sample and the composition with the solid phase; and
(c) washing the solid phase.

3. The method of claim 1 or 2, wherein the target antibody, the affinity antibody, and/or the detection antibody comprise a chimeric human-murine monoclonal IgG1-kappa antibody.

4. The method of any one of claims 1-3, wherein the target antibody, the affinity antibody, and/or the detection antibody comprise SEQ ID NOs: 1 and 4.

5. The method of any one of claims 2-4, wherein the solid phase is a plate.

6. The method of any one of claims 1-5, wherein the first affinity label is biotin.

7. The method of any one of claims 1-6, wherein the second affinity label is streptavidin.

8. The method of any one of claims 1-7, wherein the detection antibody comprises a sulfo-Tag labeled anti-TNFα antibody.

9. The method of any one of claims 2-8, wherein measuring the level of the detection label comprises:
(1) contacting the solid phase with a marker configured to produce a chemiluminescent or electrochemiluminescent signal when the marker is proximal to the detection label; and
(2) detecting the chemiluminescent or electrochemiluminescent signal.

10. The method of claim 9, wherein the marker is tripropylamine (TPA), and measuring the level of the detection label further comprises applying an electrical current to the marker to produce the chemiluminescent or electrochemiluminescent signal.

11. The method of any one of claims 2-10, wherein the solid phase is blocked with a blocking buffer before the sample and the composition are contacted with the solid phase.

12. The method of any one of claims 1-11, further comprising performing steps (i) to (iii) on a control sample.

13. The method of claim 12, wherein the control sample is incubated with the composition and contacted with the second affinity label.

14. The method of any one of claims 1-13, wherein the predetermined cut-point corresponds to a level with a 5% false-positive rate.

15. The method of claim 14, wherein the 5% false-positive rate is determined by measuring the detection label in a plurality of control samples and calculating the 5% false-positive rate.

16. The method of claim 9, wherein the chemiluminescent or electrochemiluminescent signal is measured as electrochemiluminescence (ECL).

17. The method of claim 16, wherein the predetermined cut-point is an ECL value of at least 1.

18. The method of claim 16 or 17, wherein the predetermined cut-point is an ECL value of about 1.10.

19. The method of any one of claims 1-18, wherein the method has a sensitivity that is less than 6 ng/mL.

20. The method of claim 19, wherein the sensitivity is less than 4 ng/ml.

21. The method of any one of claims 1-20, wherein the method has a drug tolerance of about 100 ng/mL.

22. A method for detecting an anti-drug antibody (ADA) that binds a target antibody comprising an anti-TNFα antibody in a sample comprising:
(i) contacting a sample with one or more anti-TNFα conjugates to form a complex between the ADA and the anti-TNFα conjugates; and
(ii) measuring a level of anti-TNFα conjugates in the complex;
wherein the method detects the ADA with a sensitivity less than 5 ng/ml.

23. The method of claim 22, wherein the one or more anti-TNFα conjugates comprise an affinity antibody comprising an anti-TNFα antibody conjugated to an affinity label.

24. The method of claim 22 or 23, wherein the one or more anti-TNFα conjugates comprise a detection antibody comprising an anti-TNFα antibody conjugated to a detection label.

25. The method of claim 23, wherein the affinity label comprises biotin.

26. The method of claim 24, wherein the detection label comprises a sulfo-Tag.

27. The method of any one of claims 22-26, wherein the method detects the ADA with a sensitivity less than 3 ng/ml.

28. The method of any one of claims 22-27, wherein the method detects the ADA with a sensitivity of 2.77 ng/ml or less.

29. A method for estimating a quantity of an anti-drug antibody (ADA) that binds a target antibody comprising an anti-TNFα antibody in a sample comprising:
(i) incubating the sample with a composition comprising:
(a) an affinity antibody comprising an anti-TNFα antibody conjugated to a first affinity label, and
(b) a detection antibody comprising an anti-TNFα antibody conjugated to a detection label;
(ii) isolating ADAs that form a complex with both the affinity antibody and the detection antibody, wherein the ADA is isolated by contacting the complex with a second affinity label that binds to the first affinity label;
(iii) detecting the ADA by measuring a level of the detection label above a predetermined cut-point;
(iv) performing steps (i)-(iii) on a known sample having a known concentration of ADA; and
(v) comparing the level of the detection label in the sample to the level of the detection label in the known sample.

30. The method of claim 29, wherein the known sample is serially diluted into a plurality of titer samples, wherein comparing the level of the detection label in the sample to the level of the detection label in the known sample comprises generating a curve of the levels of the detection label in each of the titer samples and comparing the detection label in the sample to the curve.

31. A method of confirming that an anti-drug antibody (ADA) binds a target antibody comprising an anti-TNFα antibody in a sample by performing a competitive binding assay comprising performing the method of claim 1 on the sample, wherein the composition further comprises an unlabeled anti-TNFα antibody.

32. The method of claim 31, wherein the ADA is confirmed in the sample by measuring a level of the detection label that is lower by a predetermined factor than the level of the detection label when the unlabeled anti-TNFα antibody is not present.

33. A kit for detecting an anti-drug antibody (ADA) that binds a target antibody comprising an anti-TNFα antibody in a sample comprising:
(i) an affinity antibody comprising an anti-TNFα antibody conjugated to a first affinity label;
(ii) a detection antibody comprising an anti-TNFα antibody conjugated to a detection label; and
(iii) a solid phase comprising a second affinity label, wherein the second affinity label binds to the first affinity label.

34. The kit of claim 33, wherein the solid phase is a plate.

35. The kit of claim 33 or 34, wherein the second affinity label is streptavidin.

36. The kit of any one of claims 33-35, wherein the first affinity label is biotin.

37. The kit of any one of claims 33-36, wherein the detection antibody comprises a sulfo-Tag labeled anti-TNFα antibody.

38. The kit of any one of claims 33-37, further comprising a marker configured to produce a chemiluminescent or electrochemiluminescent signal where the marker is proximal to the detection label.

39. The kit of claim 38, wherein the marker is tripropylamine (TPA).

40. The kit of any one of claims 33-39, further comprising a blocking buffer.

41. The kit of any one of claims 33-40, further comprising a wash buffer.

42. A kit for estimating a quantity of an anti-drug antibody (ADA) that binds a target antibody comprising an anti-TNFα antibody in a sample comprising:
(i) an affinity antibody comprising an anti-TNFα antibody conjugated to a first affinity label;
(ii) a detection antibody comprising an anti-TNFα antibody conjugated to a detection label;
(iii) a solid phase comprising a second affinity label, wherein the second affinity label binds to the first affinity label; and
(iv) a positive control.

43. The kit of claim 42, wherein the positive control is a plurality of titer samples.

44. A kit for confirming that an anti-drug antibody (ADA) binds a target antibody comprising an anti-TNFα antibody comprising:
(i) an affinity antibody comprising an anti-TNFα antibody conjugated to a first affinity label;
(ii) a detection antibody comprising an anti-TNFα antibody conjugated to a detection label;
(iii) an unlabeled anti-TNFα antibody; and
(iv) a solid phase comprising a second affinity label, wherein the second affinity label binds to the first affinity label.

45. Use of a target antibody comprising an anti-TNFα antibody for detecting an ADA that binds the target antibody in a sample comprising:
(i) incubating the sample with a composition comprising:
(a) a first affinity antibody comprising an anti-TNFα antibody conjugated to a first affinity label, and
(b) a detection antibody comprising an anti-TNFα antibody conjugated to a detection label;
(ii) isolating ADAs that form a complex with both the first affinity antibody and the detection antibody, wherein the ADA is isolated by contacting the complex with a second affinity label that binds to the first affinity label; and
(iii) detecting the ADA by measuring a level of the detection label above a predetermined cut-point.

46. The use of claim 45, wherein isolating the ADAs comprises:
(a) providing a solid phase comprising the second affinity label;
(b) contacting the sample and the composition with the solid phase; and
(c) washing the solid phase.

47. The use of claim 45 or 46, wherein the target antibody, the affinity antibody, and/or the detection antibody comprise a chimeric human-murine monoclonal IgG1-kappa antibody.

48. The use of any one of claims 45-47, wherein the target antibody, the affinity antibody, and/or the detection antibody comprise SEQ ID NOs: 1 and 4.

49. The use of any one of claims 46-48, wherein the solid phase is a plate.

50. The use of any one of claims 45-49, wherein the first affinity label is biotin.

51. The use of any one of claims 45-50, wherein the second affinity label is streptavidin.

52. The use of any one of claims 45-51, wherein the detection antibody comprises a sulfo-Tag labeled anti-TNFα antibody.

53. The use of any one of claims 46-52, wherein measuring the level of the detection label comprises:
(1) contacting the solid phase with a marker configured to produce a chemiluminescent or electrochemiluminescent signal when the marker is proximal to the detection label; and
(2) detecting the chemiluminescent or electrochemiluminescent signal.

54. The use of claim 53, wherein the marker is tripropylamine (TPA), and measuring the level of the detection label further comprises applying an electrical current to the marker to produce the chemiluminescent or electrochemiluminescent signal.

55. The use of any one of claims 46-54, wherein the solid phase is blocked with a blocking buffer before the sample and the composition are contacted with the solid phase.

56. The use of any one of claims 45-55, further comprising performing steps (i) to (iii) on a control sample.

57. The use of claim 56, wherein the control sample is incubated with the composition and contacted with the second affinity label.

58. The use of any one of claims 45-57, wherein the predetermined cut-point corresponds to a level with a 5% false-positive rate.

59. The use of claim 58, wherein the 5% false-positive rate is determined by measuring the detection label in a plurality of control samples and calculating the 5% false-positive rate.

60. The use of claim 53, wherein the chemiluminescent or electrochemiluminescent signal is measured as electrochemiluminescence (ECL).

61. The use of claim 60, wherein the predetermined cut-point is an ECL value of at least 1.

62. The use of claim 60 or 61, wherein the predetermined cut-point is an ECL value of about 1.10.

63. The use of any one of claims 45-62, wherein the method has a sensitivity that is less than 6 ng/mL.

64. The use of claim 63, wherein the sensitivity is less than 4 ng/ml.

65. The use of any one of claims 45-64, wherein the method has a drug tolerance of about 100 ng/mL.

66. Use of one or more anti-TNFα conjugates for detecting an anti-drug antibody (ADA) that binds a target antibody comprising an anti-TNFα antibody in a sample comprising:
(i) contacting a sample with the one or more anti-TNFα conjugates to form a complex between the ADA and the anti-TNFα conjugates; and
(ii) measuring a level of anti-TNFα conjugates in the complex;
wherein the method detects the ADA with a sensitivity less than 5 ng/mL.

67. The use of claim 66, wherein the one or more anti-TNFα conjugates comprise an affinity antibody comprising an anti-TNFα antibody conjugated to an affinity label.

68. The use of claim 66 or 67, wherein the one or more anti-TNFα conjugates comprise a detection antibody comprising an anti-TNFα antibody conjugated to a detection label.

69. The use of claim 67, wherein the affinity label comprises biotin.

70. The use of claim 68, wherein the detection label comprises a sulfo-Tag.

71. The use of any one of claims 66-70, wherein the method detects the ADA with a sensitivity less than 3 ng/mL.

72. The use of any one of claims 66-71, wherein the method detects the ADA with a sensitivity of 2.77 ng/mL or less.

73. Use of a target antibody comprising an anti-TNFα antibody for estimating a quantity of an anti-drug antibody (ADA) that binds the target antibody comprising the anti-TNFα antibody in a sample comprising:
(i) incubating the sample with a composition comprising:
(a) an affinity antibody comprising an anti-TNFα antibody conjugated to a first affinity label, and
(b) a detection antibody comprising an anti-TNFα antibody conjugated to a detection label;
(ii) isolating ADAs that form a complex with both the affinity antibody and the detection antibody, wherein the ADA is isolated by contacting the complex with a second affinity label that binds to the first affinity label;
(iii) detecting the ADA by measuring a level of the detection label above a predetermined cut-point;
(iv) performing steps (i)-(iii) on a known sample having a known concentration of ADA; and
(v) comparing the level of the detection label in the sample to the level of the detection label in the known sample.

74. The use of claim 73, wherein the known sample is serially diluted into a plurality of titer samples, wherein comparing the level of the detection label in the sample to the level of the detection label in the known sample comprises generating a curve of the levels of the detection label in each of the titer samples and comparing the detection label in the sample to the curve.

75. Use of a target antibody comprising an anti-TNFα antibody for confirming that an anti-drug antibody (ADA) binds the target antibody comprising the anti-TNFα antibody in a sample by performing a competitive binding assay comprising performing the method of claim 1 on the sample, wherein the composition further comprises an unlabeled anti-TNFα antibody.

76. The use of claim 75, wherein the ADA is confirmed in the sample by measuring a level of the detection label that is lower by a predetermined factor than the level of the detection label when the unlabeled anti-TNFα antibody is not present.


SEQUENCE LISTING (20 sequences)

SEQ ID NO: 1 - protein, 450 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTV

SEQ ID NO: 3 - protein, 332 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 106 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEV

SEQ ID NO: 6 - protein, 108 aa, synthetic construct
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 718 bp, synthetic construct
  source 1..718: mol_type = other DNA
gctagcgaattccaccatgggctggtcctgcatcatcctgtttctggtggccaccgccaccggcgtgcactccgacatcctgctgacccagtcccccgccatcctgtccgtgtctcccggcgagcgggtgtccttctcctgccgggcctcccagttcgtgggctcctccatccactggtatcagcagcggaccaacggctcccctcggctgctgatcaagtacgcctccgagtccatgtccggcatcccttcccggttctccggctccggctctggcaccgacttcaccctgtccatcaacaccgtggagtccgaggatattgccgactactactgccagcagtcccactcctggcctttcaccttcggctccggcaccaacctggaggtgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 8 - DNA, 1451 bp, synthetic construct
  source 1..1451: mol_type = other DNA
gctagcgatatcgaattcttttcttttgttttgtttccaccatgggctggtcctgcatcatcctgtttctggtggccaccgccaccggcgtgcactccgaggtgaagctggaggagtctggcggcggactggtgcagcctggcggctccatgaagctgtcctgcgtggcctccggcttcatcttctccaaccactggatgaactgggtgcgccagtcccctgagaagggcctggagtgggtggccgagatccggtccaagtccatcaactccgccacccactacgccgagtccgtgaagggccggttcaccatctcccgggacgactccaagtccgccgtgtacctgcagatgaccgacctgcggaccgaggacaccggagtgtactactgctcccggaactactacggctccacctacgactactggggccagggcaccacactgaccgtgagctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagtaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 9 - protein, 8 aa, synthetic construct
GFIFSNHW

SEQ ID NO: 10 - protein, 10 aa, synthetic construct
IRSKSINSAT

SEQ ID NO: 11 - protein, 11 aa, synthetic construct
SRNYYGSTYDY

SEQ ID NO: 12 - protein, 6 aa, synthetic construct
QFVGSS

SEQ ID NO: 13 - skipped

SEQ ID NO: 14 - protein, 9 aa, synthetic construct
QQSHSWPFT

SEQ ID NO: 15 - protein, 5 aa, synthetic construct
NHWMN

SEQ ID NO: 16 - protein, 19 aa, synthetic construct
EIRSKSINSATHYAESVKG

SEQ ID NO: 17 - protein, 9 aa, synthetic construct
NYYGSTYDY

SEQ ID NO: 18 - protein, 11 aa, synthetic construct
RASQFVGSSIH

SEQ ID NO: 19 - protein, 7 aa, synthetic construct
YASESMS

SEQ ID NO: 20 - protein, 9 aa, synthetic construct
QQSHSWPFT